(19) United States
(12) Patent Application Publication
(10) Pub. No.: US 2025/0009895 A1
(43) Pub. Date: Jan. 9, 2025

(54) DRUG LOADING MONOMOLECULAR NANO POLYMER, PRODRUG, MICELLE, DRUG DELIVERY SYSTEM, PREPARATION METHOD, AND USE

(71) Applicant: JIAXING QINGZHUN MEDI-TECH CO., LTD, Tongxiang City, Jiaxing, Zhejiang (CN)

(72) Inventor: Jun LIU, Tongxiang City (CN)

(21) Appl. No.: 18/292,836

(22) PCT Filed: Jul. 27, 2022
(86) PCT No.: PCT/CN2022/108093

§ 371 Date: Jan. 26, 2024

(30) Foreign Application Priority Data
Jul. 27, 2021 (CN) 202110860356.2

(51) Int. Cl. A61K 47/64; A61K 9/107; A61K 33/243

(52) U.S. Cl. CPC A61K 47/64; A61K 9/1075; A61K 33/243

(57) ABSTRACT

A drug loading monomolecular nano polymer, wherein a plurality of polyamino acid chains are constructed, by means of a divalent platinum-containing linker (Lₚₜ), into a nonlinear framework, the terminal of at least one polyamino acid chain is linked to a hydrophilic polymer chain, the platinum atoms in the Lₚₜ participate in forming a platinum drug unit, and a side group of a polyamino acid chain is optionally grafted with a second drug unit. The described drug loading monomolecular nano polymer can be used as a prodrug, and can constitute a micelle or drug delivery system. The present invention further relates to a preparation method and a use.

### CROSS-REFERENCE TO RELATED APPLICATIONS

This application is an U.S. national phase application under 35 U.S.C. § 371 based upon international patent application No. PCT/CN2022/108093 filed on Jul. 27, 2022, which itself claims priority to the Chinese patent application No. 202110860356.2 filed with the China National Intellectual Property Administration on Jul. 27, 2021, titled “Dual-drug monomolecular nano-polymer prodrug, method for preparing the same, and use thereof”, which is hereby incorporated by reference in its entirety.

### TECHNICAL FIELD

The present application relates to the field of pharmaceutical technology and drug delivery systems, relates to a drug-loaded monomolecular nano-polymer, a prodrug, a micelle, a drug delivery system, a preparation method and use, and specifically relates to an intracellular reducing-microenvironment responsive activatable dual-drug monomolecular nano-polymer prodrug. The present application further relates to a method for preparing the intracellular reducing-microenvironment responsive activatable dual-drug monomolecular nano-polymer prodrug, and use thereof.

### BACKGROUND

Nano-formulations of drugs have advantages such as sustained or controlled release and targeted delivery. Currently, nano-formulations represent cutting-edge technology focused on precision cancer treatment. With the rapid development of nanotechnology, various bioactive molecules (such as chemical drugs, peptides, nucleic acids, etc.) can be stored in diverse nanomaterials through various methods such as molecular self-assembly. In particular, the nanomaterials can serve as “intelligent transporters” in organisms, relying on functional design of their member molecules and exquisite control of assembly structures, possessing the potential to break through biological barriers at various levels and transport bioactive molecules directionally to target sites. At present, nano-formulations have addressed many shortcomings of conventional drug formulations. For example, the toxicity and side effects of drugs have been reduced while the bioavailability of drugs at lesion sites has been improved. In the past few decades, research of nano-drug delivery systems for targeted tumor therapy has made significant progress, with several formulations, such as liposome Doxil™, albumin Abraxane™, etc. now available globally. The diameter of anti-cancer nanoparticles is in a range from 10 nm to 100 nm. Tumor blood vessels can leak large molecules, which is called the enhanced permeability and retention effect (i.e., the EPR effect). Thus, the nanoparticles can leak out from blood vessels and accumulate within tumors, and can also spread in the extracellular space.

However, the drug release behaviors of the conventional nano-formulations of drugs such as Bind-14 and NC-6300 encounter the issue that the local instantaneous drug concentrations are difficult to reach an effective level. Despite having high accumulation potential at tumor lesion sites, the conventional nano-formulations exhibit slow drug release rates, which may lead to inferior tumor killing effects compared to free small-molecule drugs. Besides, the drug release behaviors of the conventional nano-formulations also occur in blood circulation. The early leakages of drugs can reduce the bioavailability of the drugs at the target lesion sites and cause toxicity to non-target sites. In addition, the conventional nano-formulations or the self-assembled nano-formulations have the following obvious disadvantages: 1) poor colloidal stability, leading to structural dissociation under complex physiological conditions; 2) early drug leakage; and 3) complex preparation processes, such as film hydration, nanoprecipitation, etc., with the need to remove unencapsulated drug molecules and auxiliary molecules (such as organic solvents, etc.), making large-scale production difficult.

Aiming to address the issues of selective controlled drug release in tumor microenvironment and early drug leakage, it is feasible to construct a prodrug system through chemical bonding of drug molecules. A prodrug refers to a type of drug which, after chemical structural modification, is inactive or less active in vitro, but can release active drugs through physical, chemical, or enzymatic activation in the body, thereby exerting therapeutic efficacy. The prodrug-based design strategies not only prevent the early drug leakage but also enable rapid drug release in response to tumor microenvironment or intracellular microenvironment.

Existing researches have involved bonding chemotherapeutic drugs to polymer macromolecules to obtain polymer-prodrug molecules, which then self-assemble into nanoparticles. For example, polymer-prodrug nanoparticles have been disclosed in China patent documents CN109908084A, CN101203549B, CN100457185C, and CN100344293C, and the master's thesis, “Preparation of Hydroxycamptothecin MePEG-PLA Nanoparticles and In vitro Anti-tumor Research”, Jin Tao, Zhejiang Chinese Medical University, May 1, 2013. However, these nanoparticles also have the problems including structural dissociation in blood circulation, instability in vivo, complexities in preparation process, intolerance to ultrasound, freeze-drying and rehydration, etc.

### SUMMARY

An object of the present application is to provide a drug-loaded monomolecular nano-polymer including a plurality of poly(amino acid) chains. The plurality of poly(amino acid) chains forms a non-linear backbone through interchain covalent bonding with a plurality of divalent linking groups L_{Pt}. An end of at least one of the poly(amino acid) chains is connected with a hydrophilic polymer chain. A linear backbone of the divalent linking group L_{Pt} includes a platinum atom, and the platinum atom is a component of a platinum-based drug unit. The platinum-based drug unit is a residue of either a platinum-based drug active ingredient or a prodrug of the platinum-based drug active ingredient.

Optionally, a side group of the poly(amino acid) chain is grafted with a second drug unit, and the second drug unit is a residue of either an anti-tumor drug active ingredient or a prodrug of the anti-tumor drug active ingredient.

In the drug-loaded monomolecular nano-polymer, the plurality of poly(amino acid) chains forms the non-linear backbone through the platinum-containing divalent linking groups (L_{Pt}). The end of at least one poly(amino acid) chain is connected with a hydrophilic polymer chain. The platinum atom in the divalent linking group (L_{Pt}) is a component of the platinum-based drug unit. The platinum-based drug unit can be a residue of either a platinum-based drug active ingredient or a prodrug thereof. The drug-loaded monomolecular nano-polymer can be controlled to have a branched three-dimensional structure with an appropriate cross-linking degree by adjusting the distribution density of L_{Pt}, and, in combination with the design of the hydrophilic polymer chain at the end of the poly(amino acid) chain, can form a monomolecular nano-polymer micelle with a core-shell structure in an aqueous medium without the need for self-assembly. The hydrophilic polymer chain is distributed as the shell of the micelle, and the drug component is encapsulated within the core of the micelle. The drug-loaded monomolecular nano-polymer can carry the platinum-based drug unit alone, forming a platinum-based single-drug monomolecular nano-polymer. The drug-loaded monomolecular nano-polymer can be further grafted with a second drug unit, thereby forming a dual-drug monomolecular nano-polymer. The second drug unit can be a residue of either an anti-tumor drug active ingredient or a prodrug thereof, and can be grafted onto the side group of the poly(amino acid) chain. The ratio of the platinum-based drug units to the second drug units can be flexibly adjusted by controlling the feed amounts of the corresponding monomers. The distribution density of L_{Pt} can be adjusted by controlling the ratio of non-branched amino acid monomers to a L_{Pt}-branched amino acid monomer. In the non-branched amino acid monomers, the proportion of the amino acid monomer containing the second drug unit can be flexibly adjusted. The drug-loaded monomolecular nano-polymer has good stability both in vivo and in vitro, excellent dispersibility, uniform particle size, non-toxicity, and no side effects. Further, the drug-loaded monomolecular nano-polymer does not release a drug active ingredient outside the cell, but can provide a responsive release of the drug active ingredient inside the cell. Besides, the preparation method of the drug-loaded monomolecular nano-polymer is easy to operate, mild, cost-effective and environment-friendly.

Another object of the present application is to provide a method for preparing the drug-loaded monomolecular nano-polymer, including a step of mixing a platinum-containing compound represented by formula (I-3), a monofunctional hydrophilic polymer represented by formula (III-3), optionally a pharmaceutical compound represented by formula (II-3), and optionally a compound represented by formula (IV-3) in an organic solvent to perform a ring-opening polymerization reaction;

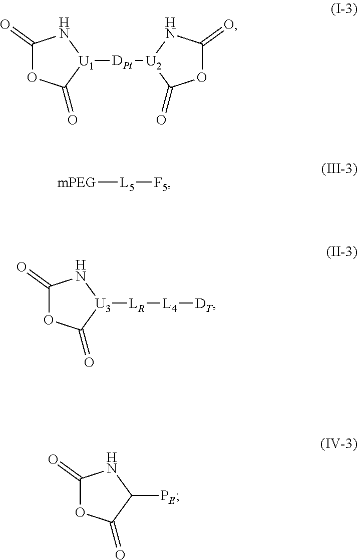

wherein U₁ and U₂ are each independently a carbon-centered trivalent group, D_{Pt} is the platinum-based drug unit, and the platinum-based drug unit can be a residue of either a platinum-based drug active ingredient or a prodrug thereof;

mPEG is a methoxy polyethylene glycol segment, which is connected to L₅ through an oxygen atom:

L₅ is independently a divalent linking group or absent;

F₅ is —NH₂, —COOH,

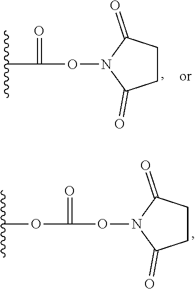 [SMILES: *C(=O)ON1C(=O)CCC1=O ; *OC(=O)ON1C(=O)CCC1=O]

preferably —NH₂;

U₃ is independently a carbon-centered trivalent group; L_{R} is independently a responsive linking group; L₄ is independently a divalent linking group or absent; D_{T} is the second drug unit, and the second drug unit can be a residue of either an anti-tumor drug active ingredient or a prodrug thereof; L_{R} is capable of having bond cleavage under external stimulation;

P_{E} is R_{E} or protected R_{E}, and is non-reactive in the ring-opening polymerization reaction; R_{E} is independently H or R₀, wherein R₀ is a terminal group including no drug unit.

Preferably, the ring-opening polymerization reaction is carried out under an anhydrous condition.

More preferably, a temperature of the ring-opening polymerization reaction is in a range from 15° C. to 40° C. Further more preferably, a time period of the ring-opening polymerization reaction is in a range from 24 hours to 96 hours.

The monomolecular nano-polymer can be prepared by a “one-pot method” through ring-opening polymerization involving bis-N-carboxy anhydride (bis-NCA). The monomolecular nano-polymer can form a micelle having a core-shell structure in an aqueous medium without the need for self-assembly, so that a drug delivery system capable of responsively releasing a drug active ingredient can be prepared for the treatment of tumor diseases.

Another object of the present application is to provide an intracellular reducing-microenvironment responsive activatable dual-drug monomolecular nano-polymer prodrug. The nano-polymer prodrug can be used as a platform for simultaneous delivery of two kinds of drug active ingredients.

Another object of the present application is to provide a method for preparing the intracellular reducing-microenvironment responsive activatable dual-drug monomolecular nano-polymer prodrug.

Another object of the present application is to provide a drug-loaded monomolecular nano-polymer micelle. The drug-loaded monomolecular nano-polymer micelle includes any one of the drug-loaded monomolecular nano-polymer as described above, the drug-loaded monomolecular nano-polymer prepared by the method as described above, the dual-drug monomolecular nano-polymer prodrug as described above, or the dual-drug monomolecular nano-polymer prodrug prepared by the method as described above. The drug-loaded monomolecular nano-polymer micelle has a core-shell structure. The shell is a hydrophilic layer made of a hydrophilic polymer chain, and the drug unit(s) is encapsulated in the core.

The drug-loaded monomolecular nano-polymer provided in the present application can form a core-shell structured nano-polymer micelle in situ during the polymerization reaction. The micelle includes a hydrophilic polymer chain in the shell, and drug unit(s) encapsulated in the core.

The platinum-based single-drug monomolecular nano-polymer provided in the present application can form a core-shell structured nano-polymer micelle in situ during the polymerization reaction. The micelle includes a hydrophilic polymer chain in the shell, and a platinum-based drug unit in the core.

The dual-drug monomolecular nano-polymer provided in the present application can form a core-shell structured nano-polymer micelle in situ during the polymerization reaction. The micelle includes a hydrophilic polymer chain in the shell, and a platinum-based drug unit and a second drug unit both in the core.

Yet another object of the present application is to provide use of the drug-loaded monomolecular nano-polymer as a prodrug. The drug-loaded monomolecular nano-polymer can enter the interior of a tumor cell and release the drug active ingredient in response to the intracellular microenvironment of the tumor cell, thereby producing cytotoxicity and inhibiting the growth of the tumor cell.

Yet another object of the present application is to provide use of the dual-drug monomolecular nano-polymer in delivery of a drug active ingredient, or use of the dual-drug monomolecular nano-polymer in preparation of a drug delivery system. The drug active ingredient(s) can be released from the platinum-based drug unit and optionally from the second drug unit. The drug active ingredient(s) can be the platinum-based drug active ingredient and optionally the anti-tumor drug active ingredient.

Yet another object of the present application is to provide use of the drug-loaded monomolecular nano-polymer as described above, use of the drug-loaded monomolecular nano-polymer prepared by the method as described above, use of the dual-drug monomolecular nano-polymer prodrug as described above, and use of the dual-drug monomolecular nano-polymer prodrug prepared by the method as described above in manufacture of a medicament for treating tumor diseases.

Yet another object of the present application is to provide a drug delivery system. The drug delivery system includes a drug-loaded monomolecular nano-polymer micelle. The drug-loaded monomolecular nano-polymer micelle includes the drug-loaded monomolecular nano-polymer as described above, or includes the drug-loaded monomolecular nano-polymer prepared by the method as described above.

Preferably, the hydrophilic polymer chain is located in the shell of the drug-loaded monomolecular nano-polymer micelle, and the platinum-based drug unit and the second drug unit are both located in the core of the drug-loaded monomolecular nano-polymer micelle.

Yet another object of the present application is to provide a drug delivery system. The drug delivery system includes a dual-drug monomolecular nano-polymer micelle. The dual-drug monomolecular nano-polymer micelle includes a poly(amino acid) connected with a hydrophilic polymer. A prodrug moiety of a platinum-based drug active ingredient and a prodrug moiety of an anti-tumor drug active ingredient are respectively bonded to α-carbons of repeating units of the poly(amino acid). Preferably, the molecule of the anti-tumor drug active ingredient includes a free hydroxyl group, a free amino group, or a combination thereof.

Yet another object of the present application is to provide use of the intracellular reducing-microenvironment responsive activatable dual-drug monomolecular nano-polymer prodrug in delivery of a drug active ingredient, or use of the intracellular reducing-microenvironment responsive activatable dual-drug monomolecular nano-polymer prodrug in preparation of a drug delivery system.

Yet another object of the present application is to provide use of a bis-NCA monomer corresponding to a platinum-based drug active ingredient and a single-NCA monomer corresponding to an anti-tumor drug active ingredient in preparation of a monomolecular nano-polymer prodrug or a drug delivery system. Preferably, the molecular structure of the anti-tumor drug active ingredient includes a free hydroxyl group or a free amino group.

Through extensive and intensive research, the applicant unexpectedly discovered that a monomolecular nano-polymer with a core-shell structure can be formed in situ from a biocompatible hydrophilic polymer including terminal amino or carboxyl groups, a bis-NCA monomer including platinum, and a single-NCA monomer corresponding to an anti-tumor drug active ingredient. The monomolecular nano-polymer can be used as a prodrug, and thus is also called a monomolecular nano-polymer prodrug. The molecule of the anti-tumor drug active ingredient preferably includes a free hydroxyl group, a free amino group, or a combination thereof, thereby is capable of being coupled to a NCA terminal group. The polymer prodrug can coupling two kinds of anti-tumor drugs, exhibits excellent stability in vivo and in vitro, has good dispersibility, uniform particle size, non-toxicity, and no side effects. Further, the polymer prodrug does not release a drug active ingredient outside the cell, but can provide a responsive release of the drug active ingredient inside the cell. Besides, the preparation method is easy to operate, mild, cost-effective and environment-friendly.

The present application further provides use of the drug-loaded monomolecular nano-polymer as described above, use of the drug-loaded monomolecular nano-polymer prepared by the method as described above, use of the dual-drug monomolecular nano-polymer prodrug as described above, use of the dual-drug monomolecular nano-polymer prodrug prepared by the method as described above, use of the drug-loaded monomolecular nano-polymer micelle as described above, or use of the drug delivery system as described above in manufacture of a medicament for treating tumor diseases.

The present application further provides an intracellular reducing-microenvironment responsive activatable dual-drug monomolecular nano-polymer prodrug with a core-shell structure, a drug delivery system including the same, a method for preparing the same, and use thereof.

The following describes the present application in various aspects. The inventions described in these aspects and any variations thereof are independent from yet interconnected to each other, collectively constituting the content of the present application.

In an aspect, the present application provides a dual-drug monomolecular nano-polymer prodrug with a core-shell structure. The core includes a structural unit of a platinum-based drug molecule, a structural unit of a drug active molecule, and a structural unit of a poly(amino acid). The structural units are connected through covalent bonds. The shell is a biocompatible hydrophilic polymer (such as polyethylene glycol, etc.) Preferably, the drug active molecule includes a free hydroxyl group, a free amino group, or a combination thereof. The dual-drug monomolecular nano-polymer prodrug of the present application can inhibit the non-specific reaction of the drug active ingredients in blood circulation, and can release the drug active ingredients in response to the intracellular reducing microenvironment after entering the cells.

In some embodiments, the present application provides a dual-drug monomolecular nano-polymer prodrug formed from a hydrophilic polymer including a terminal amino group, a bis-NCA monomer corresponding to a platinum-based drug active ingredient, and a single-NCA monomer corresponding to an anti-tumor drug active ingredient. Preferably, the molecule of the anti-tumor drug active ingredient includes a free hydroxyl group, a free amino group, or a combination thereof.

In some embodiments, the present application provides a dual-drug monomolecular nano-polymer prodrug, including a poly(amino acid) connected with a hydrophilic polymer. A prodrug moiety of a platinum-based drug active ingredient and a prodrug moiety of an anti-tumor drug active ingredient are respectively bonded to α-carbons of repeating units of the poly(amino acid). Preferably, the molecule of the anti-tumor drug active ingredient includes a free hydroxyl group, a free amino group, or a combination thereof.

In another aspect, the present application provides a drug delivery system. The drug delivery system includes a dual-drug monomolecular nano-polymer micelle. The dual-drug monomolecular nano-polymer micelle includes the dual-drug monomolecular nano-polymer as described above, or includes the dual-drug monomolecular nano-polymer prepared by the method as described above.

Preferably, the hydrophilic polymer chain is distributed in the shell of the dual-drug monomolecular nano-polymer micelle.

The platinum-based drug unit and the second drug unit are both located in the core of the dual-drug monomolecular nano-polymer micelle.

In another aspect, the present application provides a drug delivery system. The drug delivery system includes a dual-drug monomolecular nano-polymer micelle with a core-shell structure. The core includes a structural unit of a platinum-based drug molecule, a structural unit of a drug active molecule, and a structural unit of a poly(amino acid). The structural units are connected through covalent bonds. The shell is a biocompatible hydrophilic polymer (such as polyethylene glycol, etc.) Preferably, the drug active molecule includes a free hydroxyl group, a free amino group, or a combination thereof.

In some embodiments, the present application provides a drug delivery system. The drug delivery system includes a dual-drug monomolecular nano-polymer micelle. The nano-polymer micelle is formed from a hydrophilic polymer including a terminal amino group, a bis-NCA monomer corresponding to a platinum-based drug active ingredient, and a single-NCA monomer corresponding to an anti-tumor drug active ingredient. Preferably, the molecule of the anti-tumor drug active ingredient includes a free hydroxyl group, a free amino group, or a combination thereof.

In some embodiments, the present application provides a drug delivery system. The drug delivery system includes a dual-drug monomolecular nano-polymer micelle. The dual-drug monomolecular nano-polymer micelle includes a poly(amino acid) connected with a hydrophilic polymer. A prodrug moiety of a platinum-based drug active ingredient and a prodrug moiety of an anti-tumor drug active ingredient are respectively bonded to α-carbons of repeating units of the poly(amino acid). Preferably, the molecule of the anti-tumor drug active ingredient includes a free hydroxyl group, a free amino group, or a combination thereof.

In yet another aspect, the present application provides a method for preparing the dual-drug monomolecular nano-polymer prodrug, including the steps of.
(1) synthesizing a single-NCA monomer corresponding to an anti-tumor drug active ingredient under an appropriate reaction condition; preferably, the molecular structure of the anti-tumor drug active ingredient includes a free hydroxyl group or a free amino group;
(2) synthesizing a bis-NCA monomer corresponding to a platinum-based drug active ingredient under an appropriate reaction condition;
(3) reacting the single-NCA monomer obtained in step (1) and the bis-NCA monomer obtained in step (2) with a hydrophilic polymer having a terminal amino group under an appropriate reaction condition, thereby obtaining the dual-drug monomolecular nano-polymer prodrug; and
(4) separating and purifying the dual-drug monomolecular nano-polymer prodrug.

In some embodiments, the dual-drug monomolecular nano-polymer prodrug can be prepared by a “one-pot method” through ring-opening polymerization. That is, the present application provides a method for preparing a dual-drug monomolecular nano-polymer prodrug, the method including the steps of:
(1) reacting a single-NCA monomer corresponding to an anti-tumor drug active ingredient, a bis-NCA monomer corresponding to a platinum-based drug active ingredient, and a hydrophilic polymer including a terminal amino group together under an appropriate reaction condition, thereby obtaining the dual-drug monomolecular nano-polymer prodrug, preferably, the molecular structure of the anti-tumor drug active ingredient includes a free hydroxyl group or a free amino group; and
(2) separating and purifying the dual-drug monomolecular nano-polymer prodrug.

In yet another aspect, the present application provides a bis-NCA monomer corresponding to a platinum-based drug active ingredient, which is suitable for preparing the dual-drug monomolecular nano-polymer prodrug.

In yet another aspect, the present application provides a single-NCA monomer corresponding to an anti-tumor drug active ingredient, which is suitable for preparing the dual-drug monomolecular nano-polymer prodrug, and the molecular structure of the anti-tumor drug active ingredient includes a free hydroxyl group or a free amino group.

In yet another aspect, the present application provides a method for delivering a drug active ingredient to a target site, the method including preparing a monomolecular nano-polymer prodrug from the drug active ingredient, and administering an effective amount of the monomolecular nano-polymer prodrug to a patient in need thereof.

In some embodiments, the drug active ingredient includes a platinum-based drug active ingredient or a prodrug thereof.

In yet another aspect, the present application provides a method for simultaneously delivering two kinds of drug active ingredients to a target site, the method including preparing a monomolecular nano-polymer prodrug from the two kinds of target drug active ingredients, and administering an effective amount of the monomolecular nano-polymer prodrug to a patient in need thereof.

In some embodiments, the present application provides a method for simultaneously delivering two kinds of target drug active ingredients to a target site, the method including preparing a monomolecular nano-polymer prodrug from the two kinds of target drug active ingredients, and administering the prepared monomolecular nano-polymer prodrug to a patient in need thereof, wherein the two kinds of target drug active ingredients are respectively a platinum-based drug and an anti-tumor drug active ingredient, and the molecular structure of the anti-tumor drug active ingredient includes a free hydroxyl group or a free amino group.

In yet another aspect, the present application provides use of a single-NCA monomer corresponding to an anti-tumor drug active ingredient and a bis-NCA monomer corresponding to a platinum-based drug active ingredient in preparation of a drug delivery system for delivering the drug active ingredients to a target site. Preferably, the molecular structure of the anti-tumor drug active ingredient includes a free hydroxyl group or a free amino group.

In some embodiments, the present application provides use of a single-NCA monomer corresponding to an anti-tumor drug active ingredient and a bis-NCA monomer corresponding to a platinum-based drug active ingredient in preparation of a dual-drug monomolecular nano-polymer prodrug micelle. Preferably, the anti-tumor drug active ingredient includes a free hydroxyl group or a free amino group.

In yet another aspect, the present application provides use of a dual-drug monomolecular nano-polymer prodrug in manufacture of a medicament for treating corresponding diseases. The dual-drug monomolecular nano-polymer prodrug includes a poly(amino acid) connected with a hydrophilic polymer. A prodrug moiety of a platinum-based drug active ingredient and a prodrug moiety of an anti-tumor drug active ingredient are respectively bonded to α-carbons of repeating units of the poly(amino acid). Preferably, the molecule of the anti-tumor drug active ingredient includes a free hydroxyl group, a free amino group, or a combination thereof.

In yet another aspect, the present application provides a combined medication method for treating a tumor in a patient in need thereof, including administering a therapeutically effective amount of a monomolecular nano-polymer drug to the patient. The monomolecular nano-polymer drug includes a poly(amino acid) connected with a hydrophilic polymer. A prodrug moiety of a platinum-based drug active ingredient and a prodrug moiety of an anti-tumor drug active ingredient are respectively bonded to α-carbons of repeating units of the poly(amino acid). Preferably, the molecule of the anti-tumor drug active ingredient includes a free hydroxyl group, a free amino group, or a combination thereof.

The applicant unexpectedly discovered through experiments and research that the nano-polymer prodrug or the polymer prodrug nano-micelle possesses not only the advantages of the nano-formulation (such as long blood circulation time, low liver uptake, targeted accumulation potential at a lesion site, etc.), but also the advantages of a prodrug (such as reducing early deactivation of the active drug, precise drug activation, etc.). This ultimately facilitates increasing the spatiotemporal concentration of the active drug at the target site, thereby improving the efficacy of drug and reducing potential toxicity or side effects at non-target sites.

The polymer prodrug nano-micelle of the present application has better stability compared to the conventional polymer prodrug nano-micelles. Without being limited by a specific theory, the enhanced stability of the polymer prodrug nano-micelle of the present application may be attributed to factors including: fully chemical bonding of drug, which withstands physical treatments such as centrifugation, ultrafiltration, hydrothermal treatment, ultrasonication, etc., which ensures stability of chemical composition of the micelle; formation of the core due to the hydrophobic interaction between drugs such as camptothecin, which ensures the core-shell micelle structure; and cross-linking of poly(amino acid)s via platinum bridging, realizing cross-linking in the core, and thus enhancing the structural stability of the micelle.

In addition, the preparation method of the polymer prodrug nano-micelle of the present application also possesses technical advantages. Without being limited by a specific theory, the technical advantages may be attributed to factors including: fully chemical bonding of drug, which allows for precise control of the drug loading amount, overcoming the challenge of batch stability in a self-assembly system; absence of free drugs; being capable of solution storage; each nanoparticle being a single molecule, simplifying the freeze-drying and rehydration with low technical requirements; and the synthesis of the nano micelle through the “one-pot method”, without the need for complicated preparation and purification processes such as film hydration, nanoparticle precipitation, etc.

Besides, the polymer prodrug nano-micelle of the present application has advantages in drug loading and drug release. Without being limited by a specific theory, the advantages in drug loading and drug release may be attributed to factors including: fully chemical bonding of drug, ensuring no drug leakage in blood circulation and extracellular matrix; intracellular responsive release, which on the one hand, increases the spatiotemporal concentration of drug, strengthening the drug efficacy, and thus addressing the shortcoming of the passive and slow drug release of a self-assembled nano-formulation; on the other hand, the dual drugs both respond to the same intracellular microenvironment, achieving synergistical drug release, thereby maximizing the efficacy of the dual drugs.

Finally, the polymer prodrug nano-micelle of the present application can provide dual drugs, achieving the following advantages: The targets of the dual drugs are different, thus overcoming drug resistance. The intracellular drug-resistant glutathione can be depleted, thereby further overcoming the drug resistance. The platinum-based drug and another anti-tumor drug (such as camptothecin) respectively generate anti-tumor activity through different mechanisms, exhibiting excellent synergistic effects.

One or more embodiments of the present application will be described in detail with reference to the accompanying drawings and description below. Other features, objects, and advantages of the present application will become apparent from the description, drawings and claims.

### BRIEF DESCRIPTION OF THE DRAWINGS

In order to illustrate the embodiments of the present disclosure more clearly and to understand the present application and its beneficial effects more completely, the drawings used in the embodiments will be described briefly. Apparently, the following described drawings are merely for some embodiments of the present disclosure, and other drawings can be derived by those of ordinary skill in the art without any creative effort.

FIG. 1 is a schematic view showing a composition of a dual-drug (a platinum-based drug unit+a second drug unit) monomolecular nano-polymer prodrug micelle of the present application.

FIG. 2 is a schematic view showing a composition of a dual-drug (cisplatin+camptothecin) monomolecular nano-polymer prodrug micelle of the present application.

In FIG. 3A to FIG. 3D respectively show mass spectra of intermediates used for synthesis of a product of the present application.

In FIG. 4A to FIG. 4E respectively show ¹H NMR spectra of intermediates used for synthesis of a product of the present application.

In FIG. 5A and FIG. 5B respectively show molecular weight size-exclusion chromatograms of a dual-drug (cisplatin+camptothecin) monomolecular nano-polymer prodrug and an intermediate thereof of the present application.

FIG. 6 is a diagram showing dynamic light scattering (DLS) results for characterizing particle sizes and polydispersity indexes of dual-drug (cisplatin+camptothecin) monomolecular nano-polymer prodrug micelles of the present application and control micelles.

FIG. 7A and FIG. 7B show transmission electron microscopy images of: (VII-A) dual-drug (cisplatin+camptothecin) monomolecular nano-polymer prodrug micelles of the present application and (VII-B) control micelles.

FIG. 8A and FIG. 8B show transmission electron microscopy images of: (VIII-A) dual-drug (cisplatin+camptothecin) monomolecular nano-polymer prodrug micelles of the present application and (VII-B) control micelles, before and after freeze-drying.

FIG. 9A and FIG. 9B show colloidal dynamics characteristics of: (A) dual-drug (cisplatin+camptothecin) monomolecular nano-polymer prodrug micelles of the present application and (B) control micelles, tested by fluorescence correlation spectroscopy.

FIG. 10 shows a small-angle X-ray scattering diagram of dual-drug (cisplatin+camptothecin) monomolecular nano-polymer prodrug micelles in the present application.

FIG. 11A and FIG. 11B show dynamic light scattering diagrams characterizing particle size distributions of (A) dual-drug (cisplatin+camptothecin) monomolecular nano-polymer prodrug micelles of the present application and (B) control micelles.

FIG. 12 shows high-performance liquid chromatograms of dual-drug (cisplatin+camptothecin) monomolecular nano-polymer prodrug micelles of the present application and control micelles.

FIG. 13A and FIG. 13B show drug release behaviors of dual-drug (cisplatin+camptothecin) monomolecular nano-polymer prodrug micelles of the present application.

FIG. 14A to FIG. 14C show in vitro simulated drug release behaviors of control micelles.

FIG. 15 shows cytotoxicity of dual-drug (cisplatin+camptothecin) monomolecular nano-polymer prodrug micelles of the present application and control micelles.

FIG. 16A and FIG. 16B show pharmacokinetic study results of: (A) dual-drug (cisplatin+camptothecin) monomolecular nano-polymer prodrug micelles of the present application and (B) control micelles.

FIG. 17 shows in vivo drug accumulation test results of the dual-drug (cisplatin+camptothecin) monomolecular nano-polymer prodrug micelles of the present application and control micelles.

FIG. 18 shows tumor inhibition test results of dual-drug (cisplatin+camptothecin) monomolecular nano-polymer prodrug micelles of the present application and control micelles.

FIG. 19 shows a ¹H NMR spectrum of a dual-drug (cisplatin+camptothecin) monomolecular nano-polymer prodrug of the present application.

FIG. 20 shows a dynamic light scattering diagram characterizing particle size and polydispersity index of dual-drug (cisplatin+paclitaxel) monomolecular nano-polymer prodrug micelles of the present application.

FIG. 21 shows a drug release behavior of dual-drug (cisplatin+paclitaxel) monomolecular nano-polymer prodrug micelles of the present application.

FIG. 22A and FIG. 22B show transmission electron microscopy images of dual-drug (cisplatin+paclitaxel) monomolecular nano-polymer prodrug micelles of the present application.

FIG. 23 shows a dynamic light scattering diagram characterizing particle size and polydispersity index of dual-drug (cisplatin+resiquimod) monomolecular nano-polymer prodrug micelles of the present application.

FIG. 24 shows a drug release behavior of dual-drug (cisplatin+resiquimod) monomolecular nano-polymer prodrug micelles of the present application.

FIG. 25A and FIG. 25B shows transmission electron microscopy images of dual-drug (cisplatin+resiquimod) monomolecular nano-polymer prodrug micelles of the present application.

FIG. 26A and FIG. 26B shows test results of: (A) gel permeation chromatography (SEC) and (B) dynamic light scattering (DLS) of a platinum-based single-drug (cisplatin) monomolecular nano-polymer in Example 10 of the present application.

FIG. 27A and FIG. 27B show transmission electron microscopy (TEM) images of platinum-based single-drug monomolecular nano-polymer micelles in Example 10 of the present application: (A) before freeze-drying and rehydration and (B) after freeze-drying and rehydration.

FIG. 28 shows a mass spectrum of NCA-DACHPt-NCA prepared in Example 11 of the present application.

FIG. 29A and FIG. 29B shows test results of: (A) gel permeation chromatography (SEC) and (B) dynamic light scattering (DLS) of a platinum-based single-drug (DACHPt) monomolecular nano-polymer in Example 11 of the present application.

FIG. 30A and FIG. 30B shows transmission electron microscopy (TEM) images of platinum-based single-drug monomolecular nano-polymer micelles in Example 11 of the present application: (A) before freeze-drying and rehydration, and (B) after freeze-drying and rehydration.

FIG. 31 shows a ¹H NMR spectrum of PTX-ss-NCA prepared in Example 12 of the present application.

FIG. 32A and FIG. 32B shows DLS test results of a dual-drug (cisplatin+paclitaxel) monomolecular nano-polymer prepared in Example 12 of the present application: (A) without ultrasonic treatment, and (B) with ultrasonic treatment.

FIG. 33A and FIG. 33B shows transmission electron microscopy (TEM) images of dual-drug (cisplatin+paclitaxel) monomolecular nano-polymer micelles prepared in Example 12 of the present application: (A) before freeze-drying and rehydration, and (B) after freeze-drying and rehydration.

FIG. 34 shows drug release behaviors of dual-drug (cisplatin+paclitaxel) monomolecular nano-polymer micelles prepared in Example 12 of the present application under different conditions.

FIG. 35 shows a ¹H NMR spectrum of R848-ss-NCA prepared in Example 13 of the present application.

FIG. 36A and FIG. 36B show DLS test results of a dual-drug (cisplatin+R848) monomolecular nano-polymer prepared in Example 13 of the present application: (A) without ultrasonic treatment, and (B) with ultrasonic treatment.

FIG. 37A and FIG. 37B show transmission electron microscopy (TEM) images of dual-drug (cisplatin+R848) monomolecular nano-polymer micelles prepared in Example 13 of the present application: (A) before freeze-drying and rehydration, and (B) after freeze-drying and rehydration.

FIG. 38 shows drug release behaviors of dual-drug (cisplatin+R848) monomolecular nano-polymer micelles prepared in Example 13 of the present application under different conditions.

FIG. 39 shows a ¹H NMR spectrum of MMAE-ss-NCA prepared in Example 14 of the present application.

FIG. 40 shows a DLS test result of a dual-drug (cisplatin+MMAE) monomolecular nano-polymer prepared in Example 14 of the present application.

FIG. 41A and FIG. 41B show transmission electron microscopy (TEM) images of dual-drug (cisplatin+MMAE) monomolecular nano-polymer micelles prepared in Example 14 of the present application: (A) before freeze-drying and rehydration, and (B) after freeze-drying and rehydration.

FIG. 42 shows drug release behaviors of dual-drug (cisplatin+MMAE) monomolecular nano-polymer micelles prepared in Example 14 of the present application under different conditions.

### DETAILED DESCRIPTION

The present application has been generally described above, and will be described in detail with reference to embodiments below.

The present application will be described in detail with reference to the accompanying drawings, examples and embodiments. It should be understood that the specific embodiments and examples are only for explaining the present application, and not intended to limit the present application. The purpose of providing these embodiments and examples is to facilitate a more thorough and comprehensive understanding of the disclosure of the present application. It should also be understood that the present application can be implemented in many different forms and is not limited to the embodiments and examples described herein. Those of ordinary skill in the art can make various changes or modifications without departing from the concept of the present application, and the equivalent forms obtained are also within the protection scope of the present application. For example, the technical features illustrated or described as a part of one embodiment can be used in another embodiment in a suitable manner to form a further embodiment. Additionally, in the following description, numerous specific details are given to understand the present application more thoroughly. It is understood that the present application can be practiced without one or more of these details.

### Terminology

As used herein, the terms “and/or”, “or/and”, “as well as/or” when used in a list of two or more associated items, mean that any one of the items can be selected, and any or all combinations of the items can be selected. The any or all combinations include the combination of any two of the associated listed items, the combination of any more of the associated listed items, or the combination of all associated listed items. It should be noted that in the present application, when at least three items are connected by at least two conjunction combinations selected from “and/or”, “or/and”, “as well as/or”, the technical solutions encompass both the technical solutions that are all connected by “logical and” and the technical solutions that are all connected by “logical or”. For example, “A and/or B” includes three parallel solutions, i.e., A alone, B alone, and A+B. For another example, the technical solutions of “A, and/or, B, and/or, C, and/or, D” include any one of A, B, C, D (i.e., the technical solutions that are all connected by “logical or”), and include any or all combinations of A, B, C, D, i.e., include combinations of any two or any three of A, B, C, D, and a combination of A, B, C, and D (i.e., the technical solutions that are all connected by “logical and”).

Unless otherwise specified, “a plurality of”, “multiple”, etc. referred to in the present application mean that the number is greater than or equal to two. For example, “one or more” means that the number is one, or equal to or greater than two, and that is, can be one, two, or more.

As used herein, “combinations thereof”, “any combination thereof”, “any combination way thereof”, etc., include all suitable combinations of any two or more items of the listed items.

Herein, the term “suitable” in “suitable combination way”, “suitable way”, “any suitable way”, etc., is determined by being capable of implementing the technical solution of the present application, solving the technical problem of the present application, and realizing the expected technical effect of the present application.

Herein, “preferably”, “better”, “preferred”, “appropriate”, “or preferably”, etc. are merely for describing embodiments or examples having good effects. It should be understood that they do not constitute a limitation to the protection scope of the present application. When there is no contradiction or mutual restriction, if multiple instances of “preferably” appear in one technical solution, the multiple instances of “preferably” are independent from each other, unless otherwise specified. When “preferably” and one or more “or preferably” both appear in one technical solution, any two or more of “preferably” can be independent from each other, or can be combined to form different features.

In the present application, “further”, “furthermore”, “particularly”, etc., are used for description purposes, indicating differences in content, but should not be construed as limiting the protection scope of the present application.

As used herein, the terms “comprising”, “including”, or “having” are synonymous. They are inclusive or open-ended, and do not exclude additional members, elements, or steps which are not recited.

In the present application, when describing technical features in an open-ended way, the scope encompasses both a closed-ended technical solution consisting of the listed features and an open-ended technical solution including the listed features.

In the present application, a numerical range defined by endpoints can encompass all numerical values and fractions within the numerical range, as well as the endpoints.

In the present application, unless otherwise specified, when a numerical interval (i.e., a numerical range) is referred to, the optional values distributed within this numerical interval are considered continuous and include both endpoints of the numerical range (i.e., the minimum value and the maximum value), as well as every value between the two endpoints. The “value” within the numerical interval can be any quantitative value, such as a number, a percentage, a ratio, etc. The “numerical interval” can broadly encompass quantitative intervals, such as a percentage interval, a proportion interval, a ratio interval, etc. Unless otherwise specified, when a numerical interval is referred to as encompassing only integers within the numerical interval, it includes the integers at both endpoints of the numerical interval, and every integer between the endpoints. Furthermore, when multiple ranges are provided to describe a feature or a characteristic, these ranges can be combined. In other words, ranges disclosed herein are to be construed to include any or all sub-ranges subsumed therein, unless otherwise specified.

In the present application, for unit of a numerical range, if the unit is only mentioned after the right endpoint, it means that the units for both the left endpoint and the right endpoint are the same. For example, 50 to 1000 Da means that the unit Da applies to both the left endpoint, 50, and the right endpoint, 1000.

All prior arts mentioned in the present application are incorporated herein by reference as if each prior art is individually incorporated by reference. Unless there is a conflict with the objects and/or technical solutions of the present application, the referenced prior arts in the present application are incorporated in their entireties and for all purposes. When referencing prior arts in the present application, the definitions of relevant technical features, terms, nouns, phrases, etc. in the prior arts are also referenced. When referencing prior arts in the present application, the examples and preferred modes of the related technical features that are referenced can also be incorporated herein by reference, but only to the extent that they enable to carry out the present application. It should be understood that when the referenced content conflicts with the description of the present application, the present application shall prevail or the referenced content shall be adaptively modified according to the description of the present application.

To accurately understand the terminology used in the present application, the meanings of certain terms are specifically defined below. For the terms not specifically defined herein, they should be understood to have the same meanings as commonly understood and accepted by those skilled in the art. If the meaning of a term defined herein is inconsistent with the meaning commonly understood and accepted by those skilled in the art, the meaning of said term as defined herein shall prevail.

As used herein, the term “water-soluble polymer” refers to any pharmaceutically acceptable and biocompatible polymer that is soluble in water at room temperature.

As used herein, the term “water-soluble polymer having a terminal amino group” refers to the water-soluble polymer as defined above having an amino group (—NH₂) at one terminal end.

As used herein, the term “pharmaceutically acceptable excipient” refers to an auxiliary substance that can be included in a nano-polymer prodrug micelle composition of the present application, and the auxiliary substance does not cause significantly adverse pharmacological effects to patients. The term “pharmaceutically acceptable excipient” can be used interchangeably with the term “pharmaceutically acceptable carrier”.

As used herein, the term “therapeutically effective amount” refers to the dosage of the medicament of the present application that is sufficient to achieve the desired therapeutic effect on a disease when administrated to a subject. The “therapeutically effective amount” can be adjusted depending on the employed dosage form, symptoms and severity of the disease, age and weight of the subject being treated, etc.

As used herein, the term “patient” refers to a living organism suffering from or at a risk of having a disease that can be prevented or treated by administration of the dual-drug monomolecular nano-polymer prodrug of the present application, such as humans and mammals, preferably humans.

In the present application, the term “tumor” is understood in its broadest sense, and refers to tissue that is abnormally and excessively growing. The terms “carcinoma” and “cancer” refer to a malignant tumor.

In the present application, when mentioning a molecular weight of a water-soluble polymer (such as PEG), unless otherwise specified, the molecular weight refers to the weight average molecular weight.

In the present application, all numerical ranges encompass two endpoints thereof and any smaller range that are not explicitly recited and located within the specified range.

Conventional nano-formulations such as Bind-14 and NC-6300 are formed through self-assembly driven by physical interactions, such as hydrophobic interaction, electrostatic interaction, etc., between drug molecules and delivery molecules (amphiphilic molecules, e.g., lipid molecules, block copolymers, etc.). After the nano-formulations reach tumor lesions, the drugs are usually released through passive diffusion, which is a slow drug-release behavior, making it difficult to achieve effective local instantaneous drug concentrations. Therefore, despite having high accumulation potential at tumor lesions, the slow drug-release rates of the conventional nano-formulations make them even less effective than the free small-molecule drugs in tumor cell killing. Besides, the drug release behavior of the conventional nano-formulations also occurs in blood circulation. The early leakages of drugs will reduce the bioavailability of the drugs at the target lesion sites and cause toxicity to non-target sites. Therefore, controlling the drugs to be selectively and rapidly released at lesion sites is key to enhancing the effective treatment of tumors with the nano-formulations. There is an unmet need for nano-polymer prodrugs that are stable both in vivo and in vitro, offer improved drug-loading and drug-release performance, can be produced simply and efficiently, and are resistant to treatments such as ultrasound, freeze-drying, and rehydration

In some embodiments of the present application, the platinum included in the drug-loaded monomolecular nano-polymer or in the dual-drug monomolecular nano-polymer prodrug is tetravalent. Tetravalent platinum has an octahedral space structure and high chemical inertness, and is chemically stable in plasma and normal tissues. Thus, tetravalent platinum causes less systemic toxicity and side effects during delivery in the body. In addition, tetravalent platinum does not exhibit cross-resistance with divalent platinum. Once inside tumor cells, the highly reductive environment will reduce tetravalent platinum to release active divalent platinum species, thereby exerting cytotoxicity. In comparison with tetravalent platinum, divalent platinum has higher chemical reactivity and can bind to proteins in plasma, thus having lower bioavailability. In addition, divalent platinum can undergo detoxification reactions with biomolecules containing sulfhydryl groups to inactive the platinum-based drugs in the body. Besides, the cross-resistance of divalent platinum seriously restricts its clinical efficacy and long-term practicality.

In an aspect of the present application, a drug-loaded monomolecular nano-polymer is provided. The drug-loaded monomolecular nano-polymer includes a plurality of poly(amino acid) chains. The plurality of poly(amino acid) chains forms a non-linear backbone through interchain covalent bonding with a plurality of divalent linking groups L_{Pt}. An end of at least one of the poly(amino acid) chains is connected with a hydrophilic polymer chain. A linear backbone of the divalent linking group L_{Pt} includes a platinum atom, and the platinum atom is a component of a platinum-based drug unit. The platinum-based drug unit is a residue of either a platinum-based drug active ingredient or a prodrug of the platinum-based drug active ingredient.

Optionally, a side group of the poly(amino acid) chain is grafted with a second drug unit, and the second drug unit can be a residue of either an anti-tumor drug active ingredient or a prodrug of the anti-tumor drug active ingredient.

In some embodiments of the drug-loaded monomolecular nano-polymer, the residue of either the anti-tumor drug active ingredient or the prodrug thereof at each occurrence is independently connected to a corresponding amino acid repeating unit through a responsive linking group L_{R}, and the responsive linking group L_{R} is capable of having bond cleavage under external stimulation.

As used herein, the “poly(amino acid) chain” refers to a polymer chain formed by sequential connection of multiple amino carboxylic acid molecules through their —NH₂ terminals and —COOH terminals via —CO—NH— bonds. In some preferred embodiments, the poly(amino acid) chain consists of α-amino acid units. Further, the main chain of the poly(amino acid) chain is made of —NH—C—C(═O)—. Furthermore, the second drug unit at each occurrence is independently connected to α-carbon of a corresponding α-amino acid unit. As used herein, the “α-amino acid” refers to an amino acid having a structure of NH₂—CR_{C}R_{E}—COOH, in which R_{C} can be hydrogen or a non-hydrogen atom or group that does not affect the NCA ring-opening polymer, and R_{E} can be hydrogen or R₀, where R₀ is a terminal group that does not include a drug unit. R₀ can optionally be defined below.

As used herein, the “amino acid” refers to a compound containing at least one —NH₂ and at least one —COOH, can be a natural amino acid such as lysine or an unnatural amino acid such as ornithine. The amino acid units constituting the structural units of the poly(amino acid) chain of the present application can be α-amino acid units.

As used herein, the “non-linear” refers to a branched or cross-linked topological structure. In the present application, one bivalent linking group L_{Pt} can be covalently bonded to two poly(amino acid) chains to form two branch points. An appropriate degree of branching can be controlled by adjusting the ratio of L_{Pt} to the poly(amino acid) chains, and specifically can be controlled by adjusting the ratio of the number of L_{Pt} to the total number of amino acid units in the drug-loaded monomolecular nano-polymer. The greater the average number of L_{Pt} bonded to one poly(amino acid) chain, the more the branch points. An over low degree of branching will result in excessive flexibility, while a high degree of branching will form a cross-linked three-dimensional network, and an over high degree of cross-linking will result in increased rigidity. Therefore, both too few and too many branch points can affect the formation of micelle and the drug-release performance of the drug-loaded monomolecular nano-polymer.

As used herein, the term “hydrophilic polymer chain” or “hydrophilic polymer” refers to the polymers that can swell or be dissolved in water. As used herein, the “polymer” has at least two structural units. The molecular weight of the polymer is not particularly limited, and can be equal to or greater than 1,000 Da, or equal to or less than 1,000 Da. In the present application, “hydrophilic polymer” and “polymer with hydrophilicity” have the same meaning and can be used interchangeably. In the present application, “hydrophilic polymer chain” and “polymer chain with hydrophilicity” have the same meaning and can be used interchangeably.

In the present application, the hydrophilic polymer chain can be connected to either the N-terminus or the C-terminus of the poly(amino acid) chain.

In some embodiments, the hydrophilic polymer chain is connected to the C-terminus of the poly(amino acid) chain through an carboxamide bond (—CONH—).

In some embodiments, the hydrophilic polymer chain is connected to the N-terminus of the poly(amino acid) chain through an carboxamide bond (—NH—CO—) or a carbamate group (—NH—COO—).

In the present application, “valence state”, abbreviated as “valency”, refers to the number of chemical combinations of an atom, an atomic group, or a radical of a chemical element with other atoms. Those skilled in the art can understand the meanings of the valence-defined groups such as “monovalent group”, “divalent group”, “trivalent group”, etc.

In the present application, a “residue” of a substance generally refers to the remaining structure after the substance loses at least one atom. For example, —NH—C(CH₂CH₂CH₂CH₂NH—)—C(═O)— represents a trivalent residue of lysine. In addition, the platinum-containing substance in the state being covalently connected to two adjacent atoms (such as, oxygen) is also referred to as a residue of the platinum-containing substance. For example,

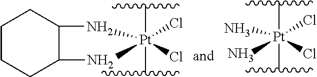

and are residues of

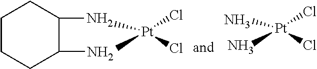

respectively.

The side groups of the amino acid units constituting the poly(amino acid) chain can be connected to L_{N} to form branch points, or can be connected to second drug units, or can be hydrogen atoms or free terminal groups R₀ that do not include any drug unit.

The hydrophilic polymer chains can be connected to the ends of the poly(amino acid) chains. During the preparation of the drug-loaded monomolecular nano-polymer, the chain length of the poly(amino acid) chains can be adjusted by introducing the hydrophilic polymer chains as terminal groups, thereby adjusting the size of the drug-loaded monomolecular nano-polymer.

The number of amino acid units connected to L_{Pt}, the number of amino acid units connected to the second drug units, the number of amino acid units with free terminal groups (i.e., the amino acid units not connected to any drug units), and the number of the hydrophilic polymer chains can be comprehensively controlled, by which the drug-loaded monomolecular nano-polymer can be controlled to have a suitable branching density, a suitable drug loading amount, a suitable ratio of different drugs, and a suitable poly(amino acid) chain length, so that the drug-loaded monomolecular nano-polymer has a suitable size, a suitable flexibility, and a suitable ratio of hydrophilic units to hydrophobic units. In combination with the position design of the hydrophilic polymer chains at the ends of the poly(amino acid) chains, the drug-loaded monomolecular nano-polymer can form a monomolecular polymer micelle with a nano-size (such as a diameter in a range from 20 to 120 nm, and further in a range from 30 to 120 nm) in an aqueous medium without the need for self-assembly. The hydrophilic polymer chains form the shell of the micelle, and the drug units are encapsulated in the core of the micelle. The sizes of the nano micelle, including the core size, the shell thickness, the particle size, the average diameter, etc., can be directly measured or calculated based on test results, by using methods including but not limited to dynamic light scattering (DLS), small angle X-ray scattering (SAXS), transmission electron microscopy (TEM), etc.

As used herein, the “aqueous medium” refers to an aqueous system, which can be water or an aqueous solution, e.g., an in vitro system such as a buffer solution, an in vitro simulation solution, a cell culture fluid, a tissue culture fluid, etc., and an in vivo system such as blood, a tissue fluid, etc.

The drug units in the drug-loaded monomolecular nano-polymer can be solely the platinum-based drug units. In this case, the drug-loaded monomolecular nano-polymer is a platinum-based single-drug monomolecular nano-polymer.

The second drug units in the drug-loaded monomolecular nano-polymer are optional. That is, the second drug units can be present or absent. In the present application, unless otherwise specified, the hydrophilicity or hydrophobicity of the second drug units is not particularly limited. That is, the second drug units can be hydrophilic or hydrophobic.

In some embodiments of the drug-loaded monomolecular nano-polymer, the second drug units are different from the platinum-based drug units, and can act on a different target.

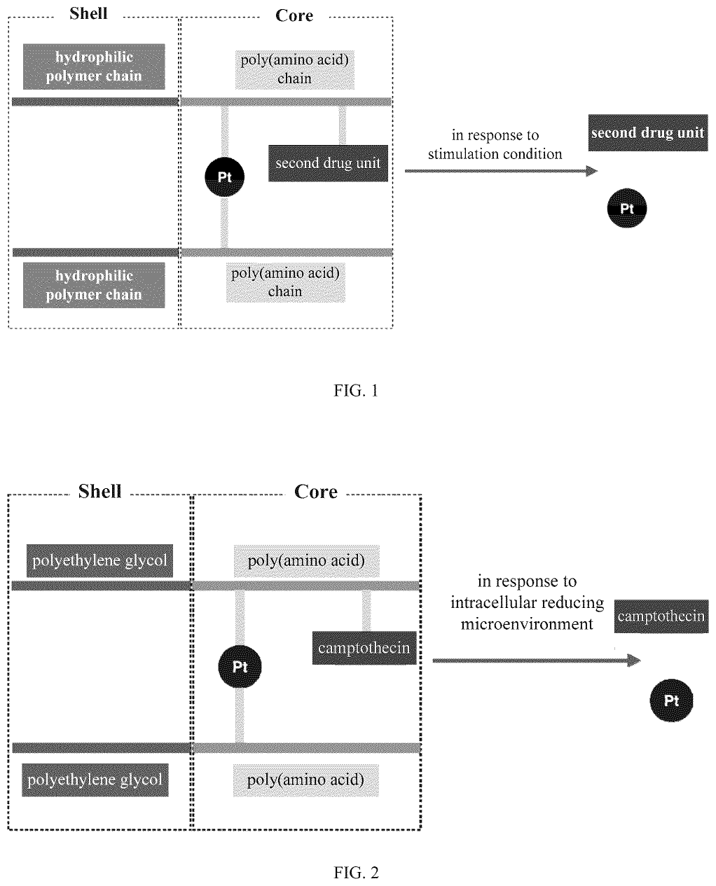

In some embodiments of the drug-loaded monomolecular nano-polymer, the drug units in the drug-loaded monomolecular nano-polymer include the platinum-based drug units and the second drug units. In this case, the drug-loaded monomolecular nano-polymer is a dual-drug monomolecular nano-polymer. FIG. 1 shows a drug release process in response to an external stimulation. FIG. 2 schematically shows a drug release process in response to an intracellular reducing-microenvironment, in which the platinum-based drug units are each a residue of cisplatin, and the second drug units are each a residue of a camptothecin active ingredient.

In the drug-loaded monomolecular nano-polymer, the plurality of poly(amino acid) chains with the platinum-containing divalent linking groups (L_{Pt}) forms the non-linear backbone. The end of at least one of the poly(amino acid) chains is connected with a hydrophilic polymer chain. The platinum atom of the L_{Pt} is a component of the platinum-based drug unit. The platinum-based drug unit can be a residue of either a platinum-based drug active ingredient or a prodrug thereof. The drug-loaded monomolecular nano-polymer can be controlled to have a branched three-dimensional structure with an appropriate cross-linking degree by adjusting the distribution density of L_{Pt}. In further combination with the design of the hydrophilic polymer chain at the end of the poly(amino acid) chain, the drug-loaded monomolecular nano-polymer can form a monomolecular nano-polymer micelle with a core-shell structure in an aqueous medium without the need for self-assembly. The hydrophilic polymer chain is distributed as the shell, and the drug component is encapsulated within the core. The drug-loaded monomolecular nano-polymer can carry the platinum-based drug unit alone, forming a platinum-based single-drug monomolecular nano-polymer. The drug-loaded monomolecular nano-polymer can be further grafted with a second drug unit, thereby forming a dual-drug monomolecular nano-polymer. The second drug unit can be a residue of either an anti-tumor drug active ingredient or a prodrug thereof, and can be grafted onto the side group of the poly(amino acid) chain. The ratio of the platinum-based drug units to the second drug units can be flexibly adjusted by controlling the feed amounts of the corresponding monomers. The distribution density of L_{Pt} can be adjusted by controlling the ratio of non-branched amino acid monomers to a L_{Pt}-branched amino acid monomer. In the non-branched amino acid monomers, the proportion of the amino acid monomer including the second drug unit can be flexibly adjusted. The drug-loaded monomolecular nano-polymer has good stability both in vivo and in vitro, excellent dispersibility, uniform particle size, non-toxicity, and no side effects. Further, the drug-loaded monomolecular nano-polymer does not release a drug active ingredient outside the cell, but can provide a responsive release of the drug active ingredient inside the cell. Besides, the preparation method is easy to operate, mild, cost-effective, and environment-friendly.

In the present application, the “L_{Pt}-branched amino acid monomer” refers to the amino acid monomer participating in forming the branch point of the non-linear backbone, such as the compound represented by formula (I-3), which carries the platinum-based drug unit.

In the present application, the “non-branched amino acid monomers” refers to the amino acid monomers not participating in forming the branch point of the non-linear backbone, such as the compound represented by formula (II-3), which carries the second drug unit, and the compound represented by formula (IV-3).

In some embodiments of the dual-drug monomolecular nano-polymer, a main chain of any poly(amino acid) chain is formed by sequential bonding of a plurality of

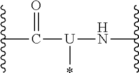

via —C(═O)—NH— bonds; in each

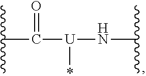

CU is independently a carbon-centered trivalent group; and each “*” terminal is independently connected to the divalent linking group L_{Pt}, or to a hydrogen atom, or to a monovalent side group R_{A}. The monovalent side group R_{A} is a side chain containing the second drug unit, or a terminal group R₀ including no drug unit.

In the present application, the “carbon-centered trivalent group” refers to a trivalent group providing a branch point from a carbon atom thereof.

In some embodiments, U at each occurrence can be CR_{C}. R_{C} can be hydrogen, or a non-hydrogen atom or group that does not affect the NCA ring-opening polymer.

In some embodiments, in each

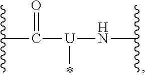

U is independently CH.

In some embodiments, each “*” terminal is independently connected to the divalent linking group L_{Pt}, or to the monovalent side group R_{A}.

In some other embodiments, each “*” terminal is independently connected to the divalent linking group L_{Pt}t, or to the side chain including the second drug unit.

In the present application, unless otherwise specified, the “alkyl” refers to a monovalent alkyl group. The “alkylene” refers to a divalent alkyl group. The “linking group” refers to an atom or a group with a valency equal to or greater than two. The “divalent linking group” refers to a linking group with a valency of 2. The “terminal group” refers to an atom or a group with a valence of 1. In the present application, the “monovalent alkyl group” refers to a residue formed by removing any one hydrogen atom from an alkane compound. The “alkylene” refers to a residue formed by removing any two hydrogen atoms from an alkane compound. The “alkane compound” refers to a saturated hydrocarbon composed of carbon atoms and hydrogen atoms. The alkane compound can be acyclic, i.e., not containing any ring, or can contain a saturated ring such as cyclohexane. Unless otherwise specified, the alkane compound is preferably acyclic.

In some embodiments, R₀ at each occurrence is independently selected from side groups of the 19 natural amino acids (excluding proline), ionic forms of suitable side groups of the 19 natural amino acids (excluding proline), and a side group of ornithine.

In some embodiments, R₀ at each occurrence is independently selected from side groups of the 19 natural amino acids (excluding proline) and a side group of ornithine.

In some embodiments, R₀ at each occurrence is independently any one selected from C₁₋₄ alkyl, -L_{A}-COOH, -L_{A}-NH₂, -L_{A}-OH, -L_{A}-SH, -L_{A}-CONH₂, -L_{A}-imidazolyl, -L_{A}-NHC(═NH)NH₂, -L_{A}-phenyl, -L_{A}-indolyl, and -L_{A}-S—C₁₋₃ alkyl. L_{A} is each independently C₁₋₆ alkylene, preferably C₁₋₄ alkylene, further preferably methylene, 1,2-ethylene, 1,3-propylene, or 1,4-butylene.

In the present application, C₁₋₄ alkyl at each occurrence is independently C₁ alkyl, C₂ alkyl, C₃ alkyl, C₄ alkyl, C₅ alkyl, or C₆ alkyl. Suitable examples include but are not limited to methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl(-CH(CH₃)C(CH₃)₃. In another example, C₁₋₃ at each occurrence is independently C₁ alkyl, C₂ alkyl, or C₃ alkyl.

In the present application, C₁₋₆ alkylene at each occurrence is independently C, alkylene, C₂ alkylene, C₃ alkylene, C₄ alkylene, C₅ alkylene, or C₆ alkylene. C₁₋₄ alkylene at each occurrence is independently C₁ alkylene, C₂ alkylene, C₃ alkylene, or C₄ alkylene. Suitable examples include but are not limited to methylene (—CH₂—), 1,1-ethyl (—CH(CH₃)—), 1,2-ethyl (—CH₂CH₂—), 1,1-propyl (—CH(CH₂CH₃)—), 1,2-propyl (—CH₂CH(CH₃)—), 1,3-propyl (—CH₂CH₂CH₂—), and 1,4-butyl (—CH₂CH₂CH₂CH₂—).

In some embodiments, R₀ at each occurrence is independently any one selected from the following groups: —CH₃, —CH(CH₃)₂, —CH₂CH(CH₃)₂, —CH(CH₃)CH₂CH₃, —CH₂CH₂SCH₃,

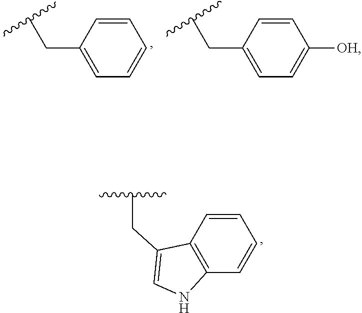 [SMILES: *Cc1ccccc1 ; *Cc1ccc(O)cc1 ; *Cc1c[nH]c2ccccc12]

—CH₂—OH —CH(OH)CH₃, —CH₂SH, —CH₂CONH₂, —CH₂CH₂CONH₂, —CH₂CH₂CH₂NH₂ or an ionic form thereof, —CH₂CH₂CH₂CH₂NH₂ or an ionic form thereof, —CH₂CH₂CH₂NHC(═NH)NH₂ or an ionic form thereof,

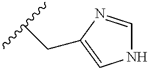 [SMILES: *Cc1c[nH]cn1]

or an ionic form thereof, —CH₂COOH or an ionic form thereof, and —CH₂CH₂COOH or an ionic form thereof.

In some embodiments, R₀ at each occurrence is independently a non-polar terminal group, such as C₁₋₆ alkyl, -L_{A}-phenyl, -L_{A}-S—C₁₋₃ alkyl, further such as —CH₃, —CH(CH₃)₂, —CH₂CH(CH₃)₂, —CH(CH₃)CH₂CH₃, —CH₂CH₂SCH₃, or

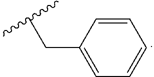 [SMILES: *Cc1ccccc1]

In some embodiments, R₀ at each occurrence is independently a polar terminal group, such as -L_{A}-COOH, -L_{A}-NH₂, -L_{A}-OH, -L_{A}-SH, -L_{A}-CONH₂, -L_{A}-imidazolyl, -L_{A}-NHC(═NH)NH₂, or -L_{A}-indolyl, further such as

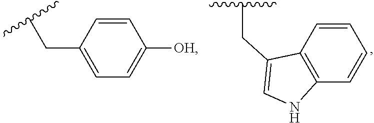 [SMILES: *Cc1ccc(O)cc1 ; *Cc1c[nH]c2ccccc12]

—CH₂—OH, —CH(OH)CH₃, —CH₂SH, —CH₂CONH₂, —CH₂CH₂CONH₂, —CH₂CH₂CH₂NH₂ or an ionic form thereof, —CH₂CH₂CH₂CH₂NH₂ or an ionic form thereof, —CH₂CH₂CH₂NHC(═NH)NH₂ or an ionic form thereof,

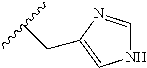 [SMILES: *Cc1c[nH]cn1]

or an ionic form thereof, —CH₂COOH or an ionic form thereof, or —CH₂CH₂COOH or an ionic form thereof.

In some embodiments, R₀ at each occurrence is independently an uncharged polar terminal group, such as —CH₂—OH, —CH(OH)CH₃, —CH₂SH, —CH₂CONH₂, or —CH₂CH₂CONH₂.

In some embodiments, R₀ at each occurrence is independently a non-polar terminal group or an uncharged polar terminal group.

In some embodiments, R₀ at each occurrence is independently a hydrophilic terminal group such as a polar terminal group, or a hydrophobic terminal group such as a non-polar terminal group. The more hydrophobic R₀ is, the denser the core formed, resulting in reduced size of the nano-polymer in an aqueous medium and a lowered drug release rate. On the contrary, the more hydrophilic R₀ is, the looser the core formed in an aqueous medium, resulting in an increased drug release rate.

In some embodiments of the drug-loaded monomolecular nano-polymer, in each molecule, the percentage of the number of platinum atoms in the divalent linking groups L_{Pt} relative to the total number of amino acid units is in a range from 10% to 100%, preferably 10% to 90%, or preferably 10% to 80%, or preferably 10% to 60%, or preferably 10% to 50%, or preferably 10% to 40%, or preferably 10% to 30%, or preferably 15% to 25%, or preferably 18% to 22%, or preferably 15% to 80%, or preferably 15% to 60%, or preferably 15% to 50%, or preferably 15% to 40%, or preferably 15% to 30%. The branch-point density of the drug-loaded monomolecular nano-polymer can be adjusted by controlling the percentage of the number of platinum atoms in the divalent linking groups L_{Pt} relative to the total number of amino acid units. In each drug-loaded monomolecular nano-polymer molecule, the percentage of the number of platinum atoms in the divalent linking groups L_{Pt} relative to the total number of amino acid units can be 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, or in an interval defined by any two of the above values.

In some embodiments of the drug-loaded monomolecular nano-polymer, in each molecule, the ratio of the number of the second drug units to the number of the platinum-based drug units is in a range from 0:1 to 10:1, preferably 0:1 to 5:1, or preferably 0:1 to 3:1, or preferably 0:1 to 1:1, or preferably 0.5:1 to 10:1, or preferably 0.5:1 to 5:1, or preferably 0.5:1 to 3:1, or preferably 1:1 to 5:1, or preferably 1:1 to 3:1, or preferably 2:1 to 3:1. The release amounts of the two different drugs can be adjusted by controlling the ratio of the number of the second drug units to the number of the platinum-based drug units. In each drug-loaded monomolecular nano-polymer molecule, the ratio of the number of the second drug units to the number of the platinum-based drug units can be 0.1:1, 0.2:1, 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.8:1, 2:1, 2.5:1, 2.6:1, 2.8:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1, or in an interval defined by any two of the above values.

In some embodiments, the drug-loaded monomolecular nano-polymer does not include the second drug unit.

In some embodiments of the drug-loaded monomolecular nano-polymer, in each molecule, the ratio of the number of the hydrophilic polymer chains to the number of the platinum-based drug units is in a range from 1:2 to 1:100, preferably 1:10 to 1:60, or preferably 1:15 to 1:45, or preferably 1:15 to 1:25. The poly(amino acid) chain length of the drug-loaded monomolecular nano-polymer can be adjusted by controlling the number of the hydrophilic polymer chains and the number of the platinum-based drug units, thereby controlling the size of the monomolecular nano-polymer and further controlling the size of the nano-polymer micelle formed in the aqueous medium. In each drug-loaded monomolecular nano-polymer molecule, the ratio of the number of the hydrophilic polymer chains to the number of the platinum-based drug units can be 1:2, 1:3, 1:4, 1:5, 1:6, 1:7, 1:8, 1:9, 1:10, 1:11, 1:12, 1:13, 1:14, 1:15, 1:16, 1:18, 1:20, 1:22, 1:24, 1:25, 1:26, 1:28, 1:30, 1:35, 1:40, 1:45, 1:55, 1:60, 1:65, 1:70, 1:75, 1:80, 1:85, 1:90, 1:95, or 1:100, or in an interval defined by any two of the above values.

In some embodiments of the drug-loaded monomolecular nano-polymer, the drug-loaded monomolecular nano-polymer includes a tetravalent structural unit represented by formula (I), a monovalent structural unit represented by formula (III), optionally a bivalent structural unit represented by formula (II), and optionally a bivalent structural unit represented by formula (IV),

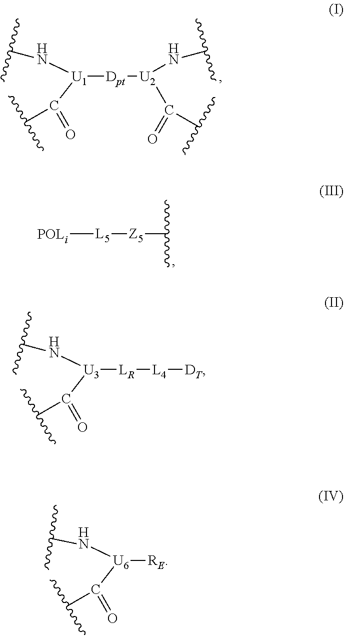

In formula (I) at each occurrence, U₁ and U₂ are each independently a carbon-centered trivalent group, and D_{Pt} is the platinum-based drug unit.

In formula (III) at each occurrence, POL; is the hydrophilic polymer chain, L₅ is independently a divalent linking group or absent, and Z₅ is independently —NH— or —C(═O)—.

In formula (II) at each occurrence, U₃ is independently a carbon-centered trivalent group, L_{R} is independently a responsive linking group, L₄ is independently a divalent linking group or absent, and D_{T} is the second drug unit. L_{R} is capable of having bond cleavage under external stimulation.

In formula (IV) at each occurrence, U₆ is independently a carbon centered trivalent group, and R_{E} is independently H or R₀, wherein R₀ is a terminal group without the second drug unit.

In some embodiments, the drug-loaded monomolecular nano-polymer includes one or both of the bivalent structural unit represented by formula (II) and the bivalent structural unit represented by formula (IV). The branch-point density of the non-linear backbone can be appropriately decreased by introducing at least one of the two structural units, so that the formed micelle can have a relatively loose core and a relatively rapid drug release rate.

In some embodiments, the drug-loaded monomolecular nano-polymer includes either the bivalent structural unit represented by formula (II) or the bivalent structural unit represented by formula (IV).

In some embodiments, the drug-loaded monomolecular nano-polymer does not include the bivalent structural unit represented by formula (II). In this case, the drug-loaded monomolecular nano-polymer is a platinum-based single-drug monomolecular nano-polymer.

In some embodiments, the drug-loaded monomolecular nano-polymer does not include the bivalent structural unit represented by formula (IV). In this case, the amino acid units are all connected with drug units including at least the platinum-based drug units.

In some embodiments, the drug-loaded monomolecular nano-polymer includes both the bivalent structural unit represented by formula (II) and the bivalent structural unit represented by formula (IV).

In some embodiments, the poly(amino acid) chain consists of the tetravalent structural unit represented by formula (I) and the bivalent structural unit represented by formula (II). In this case, the amino acid units are all connected with drug units, each of which is either the platinum-based drug unit, forming a branch point, or the second drug unit, not forming a branch point but providing a free drug-containing side chain. In these embodiments, the monomer represented by formula (IV-3) is not added as a raw material in the preparation process.

In some embodiments, the poly(amino acid) chain consists of the tetravalent structural unit represented by formula (I) and the bivalent structural unit represented by formula (IV).

In some embodiments, the poly(amino acid) chain consists of the tetravalent structural unit represented by formula (I), the bivalent structural unit represented by formula (II), and the bivalent structural unit represented by formula (IV).

In the present application, the wavy line “ [symbol] ” indicates a connection site of an atom or a group.

In some embodiments of the drug-loaded monomolecular nano-polymer,

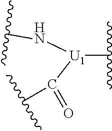

at each occurrence independently includes

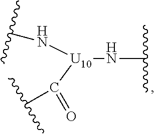

wherein U₁₀ is independently a trivalent hydrocarbon group, preferably a trivalent alkyl; more preferably,

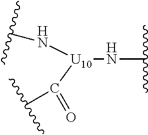

is independently a lysine unit or an ornithine unit. When

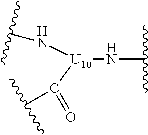

is an ornithine unit, U₁₀ is >CH—CH₂CH₂CH₂—*. When

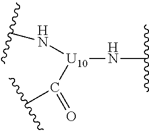

is a lysine unit, U₁₀ is >CH—CH₂CH₂CH₂CH₂—*. The “*” points to D_{Pt}.

In some embodiments,

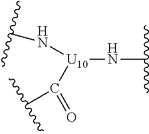

is independently an lysine unit.

In some embodiments of the drug-loaded monomolecular nano-polymer,

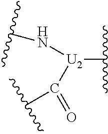

at each occurrence independently includes

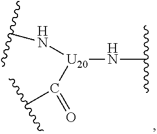

wherein U₂₀ is independently a trivalent hydrocarbon group, preferably a trivalent alkyl; more preferably,

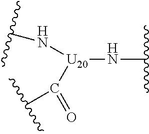

is independently a lysine unit or an ornithine unit. When

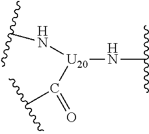

is an ornithine unit, U₂₀ is >CH—CH₂CH₂CH₂—*. When

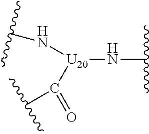

is a lysine unit, U₂₀ is >CH—CH₂CH₂CH₂CH₂—*. The “*” points to D_{Pt}.

In some embodiments,

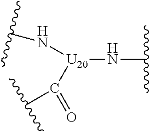

is independently a lysine unit.

In some embodiments, in each molecule, the structure of

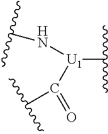

is the same as the structure of

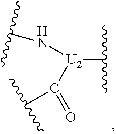

and therefore U₁ and U₂ are the same.

In some embodiments, in each molecule, U₁ and U₂ are all the same.

In some embodiments, in each molecule, the structure of

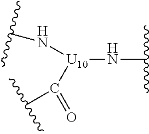

is the same as the structure of

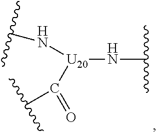

therefore U₁₀ and U₂₀ are the same.

In some embodiments, in each molecule, U₁₀ and U₂₀ are all the same.

In some embodiments of the drug-loaded monomolecular nano-polymer, D_{Pt} and adjacent groups form a main chain having a structure —C(═O)—O—Pt—O—C(═O)— or —C(═O)—NH—O—Pt—O—NH—C(═O)—.

In some embodiments of the drug-loaded monomolecular nano-polymer, D_{Pt} and adjacent groups form a main chain having a structure —C(═O)—O—Pt—O—C(═O)—.

In some embodiments of the drug-loaded monomolecular nano-polymer, formula (I) at each occurrence independently includes a structure represented by formula (I-1):

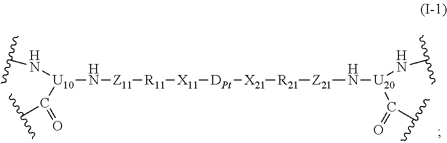

wherein U₁₀ and U₂₀ are independently as defined above;
Z₁₁, and Z₂₁ are each independently absent, —C(═O)—, or —C(═O)—O—*; or each independently —C(═O)— or —C(═O)—O—*; where “*” points to D_{Pt};
R₁₁ and R₂₁ are each independently a divalent linking group, such as a hydrocarbylene group, preferably independently an alkylene group, or preferably independently C₁₋₆ hydrocarbylene, or preferably independently C₁₋₆ alkylene; or further preferably independently methylene, 1,2-ethylene, 1,3-propylene, 1,4-butylene, 1,5-pentylene, or 1,6-hexylene; or more preferably independently methylene, 1,2-ethylene, 1,3-propylene, or 1,4-butylene; or further preferably independently 1,2-ethylene, 1,3-propylene, or 1,4-butylene; or further preferably independently 1,2-ethylene, or 1,3-propylene; or further preferably independently 1,2-ethylene; and
X_{II} and X₂₁ are each independently —C(═O)—O—* or —C(═O)—NH—O—*, preferably —C(═O)—O—*, where “*” points to D_{Pt}.

In some embodiments, Z_{II}—R_{II}—X_{II} and Z₂₁—R₂₁—X₂₁ can be each independently —C(═O)—R₀₁—C(═O)—O—*, —C(═O)—NH—R₀₁—C(═O)—O—*, —C(═O)—R₀₁—C(═O)—NH—O—*, or —C(═O)—NH—R₀₁—C═O)—NH—O—*, further can be —C═O)—R₀₁—C═O)—O—*, where “*” points to DN. The definition of R₀₁ is the same as R₁₁ or R₂₁. R₀₁ can be —(CH₂)_{q}—, wherein q can be an integer in a range from 1 to 6, such as 1, 2, 3, 4, 5, or 6, preferably 1, 2, 3, or 4, further can be 2.

In some embodiments of the drug-loading single molecule nano-polymer, D_{Pt} at each occurrence is independently a residue of a following compound: cisplatin, carboplatin, nedaplatin, oxaliplatin, or lobaplatin.

In some embodiments of the drug-loaded monomolecular nano-polymer, formula (I) at each occurrence has the same structure.

In some embodiments of the drug-loaded monomolecular nano-polymer,

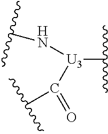

at each occurrence independently includes

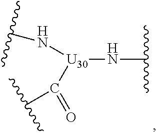

wherein U₃₀ is independently a trivalent hydrocarbon group, independently preferably a trivalent alkyl; more preferably,

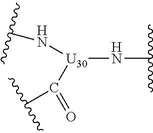

is independently a lysine unit or an ornithine unit. When

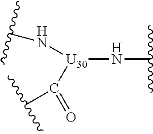

is an ornithine unit, U₃₀ is >CH—CH₂CH₂CH₂—*. When

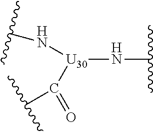

is a lysine unit, U₃₀ is >CH—CH₂CH₂CH₂CH₂—*. The “*” points to D_{Pt}.

In some embodiments,

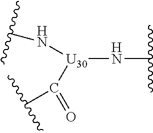

is independently a lysine unit.

In some embodiments, in each molecule, U₃ is all the same.

In some embodiments, in each molecule, U₁₀ is all the same.

In some embodiments of the drug-loaded monomolecular nano-polymer, L_{R} at each occurrence independently includes a linking group capable of having bond cleavage under at least one of the following conditions: an intracellular reducing condition, a reactive oxygen species condition, a pH condition, an enzymatic lysis condition, and a hydrolysis condition.

In some embodiments, the pH condition is a pH value less than 6.8, preferably in a range from 4.0 to 6.8.

In some embodiments, the enzymatic lysis condition includes one or both of MMP-2 enzyme and azoreductase.

In some embodiments, the hydrolysis condition is an acidic hydrolysis condition or an alkaline hydrolysis condition.

In some embodiments, L_{R} at each occurrence independently includes one or more linking groups selected from (a), (b), (c), (d), and (e):
(a) —S—S—;
(b) an oxalate group, a borate group, a thioketal group, a thioether group, a mono-selenium group, a diselenium group, a divalent tellurium group, a thiazolinone group, a boronic acid group, an oligomeric chain of 3 to 7 prolines;
(c) an acetal group, a hydrazone bond;
(d) a peptide GPLGVRG, an azo group; and
(e) —C(═O)—O—, —O—C(═O)—.

The group in (a) can respond to an intracellular reducing condition such as a glutathione environment, a reactive oxygen species condition, etc.

The groups in (b) can respond to a reactive oxygen species (ROS) condition, and thus are ROS responsive groups.

The groups in (c) can respond to a specific acidic pH condition.

The groups in (d) can be cleaved by enzymes. For example, the peptide GPLGVRG can be enzymatically cleaved by the MMP-2 enzyme. For another example, the azo group can be enzymatically cleaved by azoreductase.

The groups in (e) can be hydrolyzed.

It is to be understood that the groups in (a), (b), (c), (d), and (e) can respond to one or more stimulation conditions.

In some embodiments, L_{R} at each occurrence independently includes one or more linking groups selected from —S—S—, an oxalate group, an aryl borate group, an acetal group, a hydrazone bond, a peptide GPLGVRG, an azo group, —C(═O)—O—, and —O—C(═O)—. Preferably, the aryl borate group is a phenyl borate group.

In some embodiments, the ROS responsive groups in (b) can include one or more of the following groups: a thioketal group (—S—C(CH₃)₂—S—), a thioether bond (—S—), a mono-selenium group (—Se—), a diselenium group (—Se—Se—), a divalent tellurium group (—Te—), an oxalate group (—OC(═O)—C(═O)—O—), a thiazolinone group

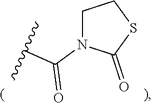 [SMILES: *C(=O)N1CCSC1=O]

a borate group
(such as

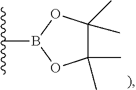 [SMILES: *B1OC(C)(C)C(C)(C)O1]

a boronic acid group (such as —B(OH)₂), or a proline oligomeric chain.

In some embodiments, the proline oligomeric chain has a structure of

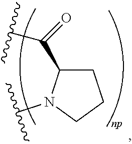

wherein np is an integer in a range from 3 to 8. In some embodiments, the number of proline units in the proline oligomeric chain is in a range from 3 to 7, such as 3, 4, 5, 6, or 7. In some embodiments, np is 7.

An example of the ROS responsive group is —Ar—OC(═O)—C(═O)—O—, wherein Ar is arylene, such as phenylene, further such as 1,4-phenylene.

In some embodiments, -L₄-D-r at each occurrence independently includes Z₄-D_{T}. Z₄ at each occurrence is independently a chemical bond, or any group selected from —C(═O)—, —O—, —S—, —O—C(═O)—*, —NH—C(═O)—*, and —NH—, wherein “*” points to D_{T}.

In some embodiments, -L₄-D_{T} at each occurrence independently has a structure of —R₃₂—Z₄-D_{T}, wherein R₃₂ at each occurrence is independently a divalent linking group, such as a hydrocarbylene group, independently preferably an alkylene group, or independently preferably C₁₋₆ hydrocarbylene, or independently preferably C₁₋₆ alkylene; or independently preferably methylene, 1,2-ethylene, 1,3-propylene, 1,4-butylene, 1,5-pentylene, or 1,6-hexylene; or independently preferably methylene, 1,2-ethylene, 1,3-propylene, or 1,4-butylene; or independently preferably 1,2-ethylene, 1,3-propylene, or 1,4-butylene; or independently preferably 1,2-ethylene, or 1,3-propylene; or independently preferably 1,2-ethylene.

In some embodiments, D_{T} and Z₄ independently form any linking group selected from —C(═O)—O—, —O—C(═O)—, —O—C(═O)—O—, —O—C(═O)—NH—, —NH—C(═O)—O—, —C(═O)—NH—, and —NH—C(═O)—.

In some embodiments, D_{T} and Z₄ independently form any linking group selected from —C(═O)—O—, —O—C(═O)—, —O—C(═O)—O—, —O—C(═O)—NH—, and —NH—C(═O)—O—.

In some embodiments, D_{T} and Z₄ independently form —O—C(═O)—O—.

In some embodiments, the linking group between L_{R} and D_{T} can be cleaved, thereby releasing the drug active ingredient or a prodrug thereof corresponding to D_{T}. Examples of the cleavable linking groups include —C(═O)—O—, —O—C(═O)—, —O—C(═O)—O—, —O—C(═O)—NH—, or —NH—C(═O)—O—.

In some embodiments, formula (II) at each occurrence has a structure represented by formula (II-1):

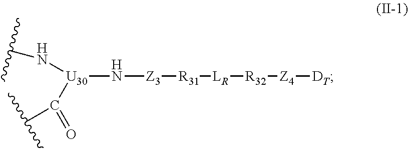

wherein U₃₀ at each occurrence is independently as defined above;
R₃₂ and Z₄ at each occurrence are independently as defined above;
Z₃ at each occurrence is independently absent, —C(═O)—, or —C(═O)—O—*, can independently be —C(═O)—, or —C(═O)—O—*, or can independently preferably be —C(═O)—O—*, or can independently preferably be —C(═O)—; where “*” points to R₃₁;
R₃₁ at each occurrence is independently a divalent linking group, such as a hydrocarbylene group, independently preferably an alkylene group, or independently preferably C₁₋₆ hydrocarbylene, or independently preferably C₁₋₆ alkylene; or independently preferably methylene, 1,2-ethylene, 1,3-propylene, 1,4-butylene, 1,5-pentylene, or 1,6-hexylene; or independently preferably methylene, 1,2-ethylene, 1,3-propylene, or 1,4-butylene; or independently preferably 1,2-ethylene, 1,3-propylene, or 1,4-butylene; or independently preferably 1,2-ethylene, or 1,3-propylene; or independently preferably 1,2-ethylene.

In some embodiments, R₃₁-L_{R}-R₃₂ at each occurrence is independently —(CH₂)ₚ₁—S—S—(CH₂)ₚ₂—, wherein p1 and p2 are each independently an integer in a range from 1 to 6, such as 1, 2, 3, 4, 5, or 6, independently preferably 1, 2, 3 or 4, or independently preferably 2 or 3.

In some embodiments, R₃₁-L_{R}-R3₂ at each occurrence is independently —(CH₂)₂—S—S—(CH₂)₂—.

In some embodiments, Z₃—R₃₁-L_{R}-R₃₂ at each occurrence is independently —C(═O)—(CH₂)₂—S—S—(CH₂)₂—*, or —C(═O)—O—(CH₂)₂—S—S—(CH₂)₂—*, wherein “*” points to D_{T}.

In some embodiments, D_{T} at each occurrence is independently a residue of a following compound: a camptothecin-based compound, resiquimod, or paclitaxel.

It should be understood that the drug active ingredient or the prodrug thereof corresponding to D_{T} has a reactive group F_{T}, or can be activated to have the reactive group F_{T}, so as to be connected with L_{R}. The reactive group F_{T} can be one or more of a hydroxyl group, a carboxyl group, an amino group, a thiol group, etc. In some embodiments, the reactive group F_{T} is a free hydroxyl group, a free amino group, or a combination thereof. When the drug active ingredient or the prodrug thereof corresponding to D_{T} includes two or more reactive groups, some of the reactive groups can be protected such that one D_{T} is connected to one L_{R}. The reactive group F_{T} can be reacted to form a covalent linking group through a conventional coupling reaction. For example, a hydroxyl group can be reacted to form an ether bond (—O—), an ester group (—O—C(═O)—), a carbonate group (—O—C(═O)—O—), etc. For example, a carboxyl group can be reacted to form an amide bond (—C(═O)—NH—). For example, an amino group can be reacted to form a divalent amino group (—NH—), a carboxamide bond (—C(═O)—NH—), a carbamate group (—NH—C(═O)—O—), etc. An appropriate Z₄ linking group can be selected depending on the structural features of the drug active ingredient or the prodrug thereof corresponding to D_{T}.

In some embodiments, the camptothecin-based compound is camptothecin, a derivative or analog thereof.

In some embodiments, the camptothecin-based compound is irinotecan, topotecan, rubitecan, chimmitecan, 9-aminocamptothecin, 9-nitrocamptothecin, or 7-ethyl-10-hydroxycamptothecin.

In some embodiments, formula (II) at each occurrence has the same structure.

In some embodiments, in formula (III), Z₅ at each occurrence is independently —NH—, —C(═O)—, or *—O—C(═O)—, wherein “*” points to L₅.

In some embodiments, in formula (III), L₅ at each occurrence is independently a divalent linking group, such as a hydrocarbylene group, independently preferably an alkylene group, or independently preferably C₁₋₆ hydrocarbylene, or independently preferably C₁₋₆ alkylene; or independently preferably methylene, 1,2-ethylene, 1,3-propylene, 1,4-butylene, 1,5-pentylene, or 1,6-hexylene; or independently preferably methylene, 1,2-ethylene, 1,3-propylene, or 1,4-butylene; or independently preferably 1,2-ethylene, 1,3-propylene, or 1,4-butylene; or independently preferably 1,2-ethylene, or 1,3-propylene; or independently preferably 1,2-ethylene.

In some embodiments, in formula (III) at each occurrence, L₅ and Z₅ are the same.

In some embodiments, in formula (III), POL; at each occurrence independently includes any hydrophilic polymer chain selected from polyethylene glycol, poly(propylene glycol), a copolymer of ethylene glycol and propylene glycol, poly(ethoxylated polyol), poly(alkenol), poly(vinylpyrrolidone), poly(hydroxyalkyl methacrylamide), poly(hydroxyalkyl methacrylate), polysaccharide, poly(α-hydroxyacid), poly(vinyl alcohol), polyphosphazene, polyoxazoline, poly(N-acryloylmorpholine), and any combination thereof.

In some embodiments, in formula (III), a molecular weight of the hydrophilic polymer chain is in a range from 50 Da to 100 kDa, preferably 100 Da to 80 kDa, or preferably 500 Da to 50 kDa, or preferably 500 Da to 10 kDa, or preferably 500 Da to 8000 Da, or preferably 500 Da to 6000 Da, or preferably 500 Da to 5000 Da, or preferably 1000 Da to 50 kDa, or preferably 1000 Da to 10 kDa, or preferably 1000 Da to 8000 Da, or preferably 1000 Da to 6000 Da, or preferably 1000 Da to 5000 Da. The molecular weight of the hydrophilic polymer chain can be about 500 Da, about 600 Da, about 700 Da, about 750 Da, about 800 Da, about 850 Da, about 900 Da, about 950 Da, about 1000 Da, about 1100 Da, about 1200 Da, about 1300 Da, about 1400 Da, about 1500 Da, about 1600 Da, about 1800 Da, about 2000 Da, about 2200 Da, about 2400 Da, about 2500 Da, about 3000 Da, about 3500 Da, about 4000 Da, about 4500 Da, about 5000 Da, about 5500 Da, about 6000 Da, about 6500 Da, about 7000 Da, about 7500 Da, about 8000 Da, about 8500 Da, about 9000 Da, or about 10000 Da, or in an interval defined by any two of the above values. The “about” means ±10%, ±5%, or ±2%, or 0.

In some embodiments, in formula (III). POLᵢ at each occurrence independently includes a polyethylene glycol segment. Preferably, the polyethylene glycol segment is methoxypolyethylene glycol (mPEG). Or preferably, a molecular weight of the polyethylene glycol segment is in a range from 50 Da to 100 kDa, or preferably 100 Da to 80 kDa, or preferably 500 Da to 50 kDa, or preferably 500 Da to 10 kDa, or preferably 500 Da to 8000 Da, or preferably 500 Da to 6000 Da, or preferably 500 Da to 5000 Da, or preferably 1000 Da to 50 kDa, or preferably 1000 Da to 10 kDa, or preferably 1000 Da to 8000 Da, or preferably 1000 Da to 6000 Da, or preferably 2000 Da to 6000 Da, or preferably 4000 Da to 6000 Da, or preferably 1000 Da to 5000 Da; or preferably about 500 Da, about 600 Da, about 800 Da, about 1000 Da, about 1100 Da, about 1200 Da, about 1500 Da, about 1600 Da, about 2000 Da, about 2200 Da, about 2500 Da, about 3000 Da, about 3500 Da, about 4000 Da, about 4400 Da, about 4500 Da, about 5000 Da, about 5500 Da, about 6000 Da, about 6500 Da, about 7000 Da, about 8000 Da, about 9000 Da, about 10 kDa, about 12 kDa, about 15 kDa, about 20 kDa, about 25 kDa, about 30 kDa, about 35 kDa, about 40 kDa, or about 40 kDa. The “about” means ±10%, ±5%, or ±2%, or 0.

In formula (III), the “molecular weight” of each POLᵢ independently represents a weight average molecular weight or a number average molecular weight.

In formula (III), the “molecular weight” of each POLᵢ independently represents a weight average molecular weight.

In formula (III), the “molecular weight” of each POLᵢ independently represents a number average molecular weight.

In some embodiments, the formula (III) at each occurrence has the same structure.

In some embodiments of the drug-loaded monomolecular nano-polymer, formula (IV) at each occurrence has a structure represented by formula (IV-1):

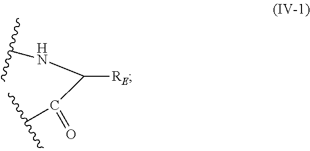

wherein R_{E} at each occurrence is independently a hydrogen atom or R₀, and R₀ is a terminal group including no drug unit.

In some embodiments, R_{E} at each occurrence is independently R₀.

In some embodiments, R₀ is as defined in any preceding embodiment.

In some embodiments, the poly(amino acid) chain includes an amino acid unit having a structure represented by formula (IV-1).

In some embodiments, formula (IV) at each occurrence has the same structure.

In some embodiments of the drug-loaded monomolecular nano-polymer, formula (I) at each occurrence has the same structure; formula (III) at each occurrence has the same L₅ and Z₅; formula (II), if present, at each occurrence has the same structure; formula (IV), if present, at each occurrence has the same structure. In this case, only a single type of raw material needs to be provided for each corresponding structural unit in preparation of the drug-loaded monomolecular nano-polymer.

In some embodiments of the drug-loaded monomolecular nano-polymer, the drug-loaded monomolecular nano-polymer includes a tetravalent structural unit represented by formula (I-2), a monovalent structural unit represented by formula (III-2), optionally a bivalent structural unit represented by formula (II-2), and optionally a bivalent structural unit represented by formula (IV-1),

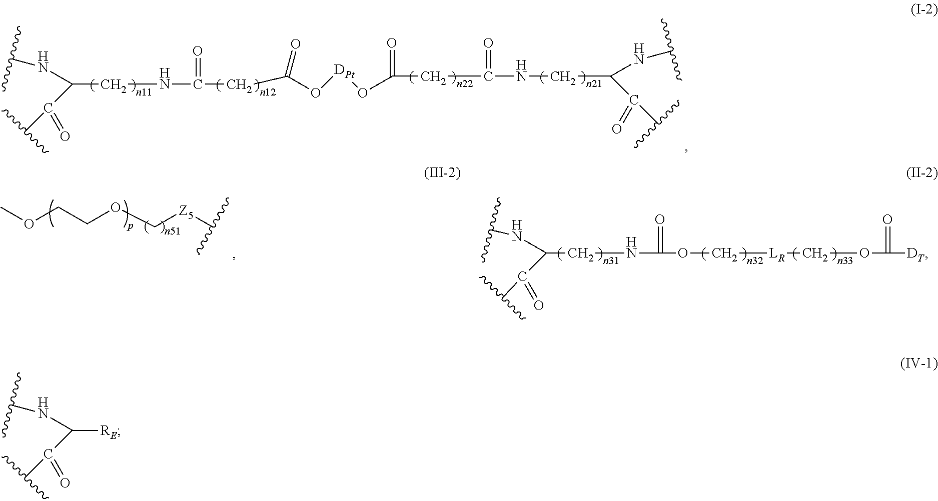

preferably,
n11 and n21 are each independently 3 or 4, and n12 and n22 are each independently 1, 2, 3, 4, or 5;
n31 is independently 3 or 4; n32 is independently 2, 3 or 4; and n33 is independently 2, 3 or 4;
n51 is independently 1, 2, 3, or 4;
p is independently a positive integer, preferably a positive integer less than or equal to 2500, or preferably a positive integer less than or equal to 2000, or preferably a positive integer less than or equal to 1500, or preferably a positive integer less than or equal to 1000, or preferably a positive integer less than or equal to 800, or preferably a positive integer less than or equal to 600, or preferably a positive integer less than or equal to 500, or preferably a positive integer less than or equal to 400, or preferably a positive integer less than or equal to 300, or preferably a positive integer less than or equal to 250, or preferably a positive integer less than or equal to 200, or preferably an integer in a range from 2 to 2500, or preferably an integer in a range from 3 to 2000, or preferably an integer in a range from 5 to 1500, or preferably an integer in a range from 5 to 1000, or preferably an integer in a range from 5 to 800, or preferably an integer in a range from 5 to 600, or preferably an integer in a range from 5 to 500, or preferably an integer in a range from 5 to 400, or preferably an integer in a range from 5 to 300, or preferably an integer in a range from 5 to 250, or preferably an integer in a range from 5 to 200, further preferably an integer in a range from 5 to 1500, further preferably an integer in a range from 5 to 1000, or preferably an integer in a range from 5 to 800, or preferably an integer in a range from 10 to 600, or preferably an integer in a range from 10 to 500, or preferably an integer in a range from 10 to 400, or preferably an integer in a range from 10 to 300, or preferably an integer in a range from 10 to 250, or preferably an integer in a range from 10 to 200, or preferably an integer in a range from 20 to 600, or preferably an integer in a range from 20 to 500, or preferably an integer in a range from 20 to 400, or preferably an integer in a range from 20 to 300, or preferably an integer in a range from 20 to 250, or preferably an integer in a range from 20 to 200, or preferably an integer in a range from 50 to 500, or preferably an integer in a range from 50 to 400, or preferably an integer in a range from 50 to 300, or preferably an integer in a range from 50 to 250, or preferably an integer in a range from 50 to 200, or preferably an integer in a range from 100 to 500, or preferably an integer in a range from 100 to 400, or preferably an integer in a range from 100 to 300, or preferably an integer in a range from 100 to 250, or preferably an integer in a range from 100 to 200, or preferably an integer in a range from 100 to 150.

In some embodiments, p is preferably a positive integer less than or equal to 500, or preferably a positive integer less than or equal to 400, or preferably a positive integer less than or equal to 300, or preferably a positive integer less than or equal to 250, or preferably a positive integer less than or equal to 200, or an integer in a range from 5 to 500, or preferably an integer in a range from 5 to 400, or preferably an integer in a range from 5 to 300, or preferably an integer in a range from 5 to 250, or preferably an integer in a range from 5 to 200, or preferably an integer in a range from 10 to 500, or preferably an integer in a range from 10 to 400, or preferably an integer in a range from 10 to 300, or preferably an integer in a range from 10 to 250, or preferably an integer in a range from 10 to 200, or preferably an integer in a range from 20 to 500, or preferably an integer in a range from 20 to 400, or preferably an integer in a range from 20 to 300, or preferably an integer in a range from 20 to 250, or preferably an integer in a range from 20 to 200, or preferably an integer in a range from 50 to 500, or preferably an integer in a range from 50 to 400, or preferably an integer in a range from 50 to 300, or preferably an integer in a range from 50 to 250, or preferably an integer in a range from 50 to 200, or preferably an integer in a range from 100 to 500, or preferably an integer in a range from 100 to 400, or preferably an integer in a range from 100 to 300, or preferably an integer in a range from 100 to 250, or preferably an integer in a range from 100 to 200, or preferably an integer in a range from 100 to 150.

In some embodiments, p can be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 70, 80, 90, 100, 110, 120, 130, 140, 150, 160, 170, 180, 190, 200, 210, 215, 220, 225, 227, 240, 250, 260, 280, 300, 350, 400, 450, or 500, or an integer in an interval defined by any two of the above values.

In some embodiments, p can be an integer within any range selected from 110 to 120, 100 to 120, 100 to 130, 100 to 140, 100 to 150, 90 to 120, 90 to 130, 90 to 140, 90 to 150, 80 to 120, 80 to 130, 80 to 140, 80 to 150, etc.

In some embodiments, p can be an integer within any range selected from 5 to 115, 5 to 114, 5 to 110, 5 to 100, 5 to 90, 5 to 88, 5 to 78, 5 to 78, 5 to 777, 5 to 66, 5 to 65, 5 to 60, 5 to 55, 5 to 50, 5 to 45, 5 to 44, 5 to 40, 5 to 35, 5 to 34, 5 to 33, 5 to 30, 5 to 25, 5 to 20, 6 to 115, 6 to 114, 6 to 110, 6 to 100, 6 to 90, 6 to 88, 6 to 78, 6 to 78, 6 to 777, 6 to 66, 6 to 65, 6 to 60, 6 to 55, 6 to 50, 6 to 45, 6 to 44, 6 to 40, 6 to 35, 6 to 34, 6 to 33, 6 to 30, 6 to 25, 6 to 20, 8 to 115, 8 to 114, 8 to 110, 8 to 100, 8 to 90, 8 to 88, 8 to 78, 8 to 78, 8 to 777, 8 to 66, 8 to 65, 8 to 60, 8 to 55, 8 to 50, 8 to 45, 8 to 44, 8 to 40, 8 to 35, 8 to 34, 8 to 33, 8 to 30, 8 to 25, 8 to 20, 10 to 115, 10 to 114, 10 to 110, 10 to 100, 10 to 90, 10 to 88, 10 to 78, 10 to 78, 10 to 777, 10 to 66, 10 to 65, 10 to 60, 10 to 55, 10 to 50, 10 to 45, 10 to 44, 10 to 40, 10 to 35, 10 to 34, 10 to 33, 10 to 30, 10 to 25, 10 to 20, 15 to 115, 15 to 114, 15 to 110, 15 to 100, 15 to 90, 15 to 88, 15 to 78, 15 to 78, 15 to 777, 15 to 66, 15 to 65, 15 to 60, 15 to 55, 15 to 50, 15 to 45, 15 to 44, 15 to 40, 15 to 35, 15 to 34, 20 to 115, 20 to 114, 20 to 110, 20 to 100, 20 to 90, 20 to 88, 20 to 78, 20 to 78, 20 to 777, 20 to 66, 20 to 65, 20 to 60, 20 to 55, 20 to 50, 20 to 45, 20 to 44, 20 to 40, 20 to 35, 20 to 34, etc.

The bivalent structural unit represented by formula (IV-1) is as defined above.

In some embodiments, n11 and n21 are each independently 3 or 4, and n11 and n21 are further each independently 4.

In some embodiments, n12 and n22 are each independently 2, 3, 4 or 5, or each independently 2 or 3, or each independently 2, or each independently 3.

In some embodiments, n31 is independently 3 or 4, and further independently 4.

In some embodiments, n32 is independently 2, 3, or 4, and further independently 2.

In some embodiments, n33 is independently 2, 3, or 4, and further independently 2.

In some embodiments, n51 is independently 1, 2, 3 or 4, further independently 2, 3 or 4, further independently 2 or 3, further independently 2, further independently 3.

In some embodiments, n1 and n21 are each independently 4, and n12 and n22 are each independently 4.

In some embodiments, n31 is independently 4; n32 is independently 2; and n33 is independently 2.

In some embodiments, n51 is independently 2, 3 or 4, and further independently 3.

In some embodiments, L_{R} is —S—S—.

In some embodiments, Z₅ is —NH—.

In some embodiments, L_{R} is —S—S—; and Z₅ is —NH—.

In some embodiments, D_{Pt} is a residue of a following compound: cisplatin, oxaliplatin, or

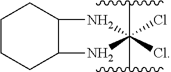

D_{T} is a residue of a following compound: camptothecin, paclitaxel, or resiquimod.

In some embodiments of the drug-loaded monomolecular nano-polymer, the molecular weight of the drug-loaded monomolecular nano-polymer can be greater than 50 kDa, further greater than 100 kDa, further in a range from 100 kDa to 5000 kDa, further in a range from 150 kDa to 5000 kDa, further in a range from 200 kDa to 5000 kDa, further in a range from 250 kDa to 5000 kDa, further in a range from 300 kDa to 5000 kDa, further in a range from 400 kDa to 5000 kDa, preferably 500 kDa to 5000 kDa, further preferably 500 kDa to 4000 kDa, further preferably 500 kDa to 3000 kDa, further preferably 500 kDa to 2500 kDa, further preferably 500 kDa to 2000 kDa, further preferably 500 kDa to 1500 kDa, further preferably 600 kDa to 1500 kDa, further preferably 800 kDa to 1200 kDa. The molecular weight can be a weight average molecular weight or a number average molecular weight. The molecular weight is preferably a weight average molecular weight or preferably a number average molecular weight. The molecular weight of the drug-loaded monomolecular nano-polymer can be 100 kDa, 150 kDa, 200 kDa, 250 kDa, 300 kDa, 400 kDa, 500 kDa, 550 kDa, 600 kDa, 650 kDa, 700 kDa, 750 kDa, 800 kDa, 850 kDa, 900 kDa, 950 kDa, 1000 kDa, 1100 kDa, 1200 kDa, 1300 kDa, 1400 kDa, 1500 kDa, 1600 kDa, 1700 kDa, 1800 kDa, 1900 kDa, 2000 kDa, 2100 kDa, 2200 kDa, 2300 kDa, 2400 kDa, 2500 kDa, 3000 kDa, 3500 kDa, 4000 kDa, 4500 kDa, or 5000 kDa, or in an interval defined by any two of the above values.

In some embodiments of the drug-loaded monomolecular nano-polymer, the number of platinum atoms in each drug-loaded monomolecular nano-polymer molecule is greater than 40, further greater than 50, or can be in a range from 50 to 5000, further in a range from 50 to 4000, further in a range from 50 to 2000, further in a range from 50 to 1500, further in a range from 50 to 1000, further in a range from 50 to 500, further in a range from 60 to 2000, further in a range from 60 to 1500, further in a range from 60 to 1000, further in a range from 60 to 500, further in a range from 80 to 2000, further in a range from 80 to 1500, further in a range from 80 to 1000, further in a range from 80 to 500, further in a range from 100 to 2000, further in a range from 100 to 1500, further in a range from 100 to 1000, further in a range from 100 to 500, further in a range from 150 to 2000, further in a range from 150 to 1500, further in a range from 150 to 1000, further in a range from 150 to 500, further in a range from 200 to 2000, further in a range from 200 to 1500, further in a range from 200 to 1000, further in a range from 200 to 500, further in a range from 250 to 2000, further in a range from 250 to 1500, further in a range from 250 to 1000, further in a range from 250 to 500, further in a range from 300 to 400. In each drug-loaded monomolecular nano-polymer molecule, the number of platinum atoms can further be 50, 60, 80, 100, 120, 150, 200, 250, 300, 350, 400, 450, 500, 550, 600, 650, 700, 750, 800, 900, 1000, 1100, 1200, 1300, 1400, 1500, 1600, 1800, 2000, 2500, or 3000, or in an interval defined by any two of the above values.

The molecular weight of the drug-loaded monomolecular nano-polymer is controlled to be in an appropriate range, so that the micelle formed therefrom can be controlled to have a size suitable for preparation of a medicament. For example, the size of the micelle in water, an aqueous solution, or an in vivo environment can be affected. Taking the drug-loaded monomolecular nano-polymer with a number average molecular weight of 1000 kDa as an example, in some embodiments, its micelle formed in water at 25° C. can have an average diameter in a range from 25 nm to 45 nm, such as about 30 nm, 32 nm, 34 nm, 44 nm, etc.

In the present application, only when specified, the particle size of the drug-loaded monomolecular nano-polymer micelle refers to an average diameter or an average particle size. Unless otherwise specified, the particle size is tested at a temperature of 20° C. to 30° C., such as 25° C.

Further, the present application provides a method for preparing a drug-loaded monomolecular nano-polymer, the method including the following step:
mixing a platinum-containing compound represented by formula (1-3), a monofunctional hydrophilic polymer represented by formula (III-3), optionally a pharmaceutical compound represented by formula (II-3), and optionally a compound represented by formula (IV-3) in an organic solvent to perform a ring-opening polymerization reaction;

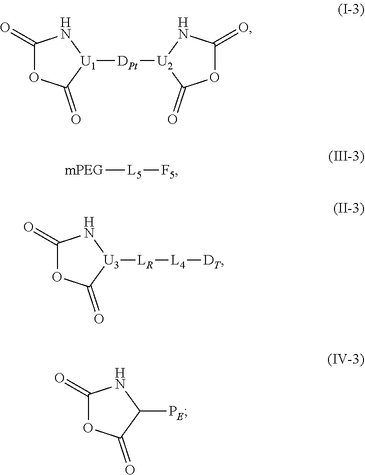

wherein P_{E} is R_{E} or protected R_{E}, and is non-reactive in the ring-opening polymerization reaction, i.e., P_{E} has no reactivity in the ring-opening polymerization reaction;
U₁, U₂, D_{Pt}, U₃, L_{R}, L₄, D_{T}, mPEG, L₅ and R_{E} are respectively as defined above;
F₅ is —NH₂, —COOH,

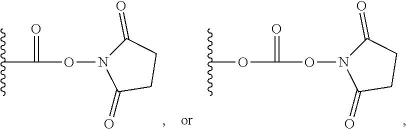 [SMILES: *C(=O)ON1C(=O)CCC1=O ; *OC(=O)ON1C(=O)CCC1=O]

preferably —NH₂.

In the present application, unless otherwise specified, mPEG is CH₃(OCH₂CH₂)ₚ—O—, wherein p is defined as above.

In some embodiments, the ring-opening polymerization reaction is carried out under an anhydrous condition.

In some embodiments, a temperature of the ring-opening polymerization reaction is in a range from 15° C. to 40° C.; preferably, a time period of the ring-opening polymerization reaction is in a range from 24 hours to 96 hours.

The platinum-containing compound represented by formula (I-3), expressed as NCA-Pt-NCA, is a bis-NCA monomer. The pharmaceutical compound represented by formula (II-3), expressed as NCA-L_{R}-D_{T}, is a single-NCA monomer. The compound represented by formula (IV-3), expressed as NCA-AA, is a single-NCA monomer. They are all NCA-functionalized amino acid monomers.

In the present application, the “NCA” refers to the functional group “N-carboxy anhydride”. The functional group “N-carboxy anhydride” refers to a functional group containing —NH—C(═O)—O—C(═O)—. Unless otherwise specified, the functional group “N-carboxy anhydride” is preferably in a cyclic structure, further preferably a five-membered ring, such as

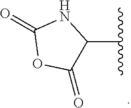 [SMILES: *C1NC(=O)OC1=O]

The monomolecular nano-polymer can be prepared by a “one-pot method” through ring-opening polymerization involving bis-N-carboxy anhydride (bis-NCA). The obtained monomolecular nano-polymer can form a micelle having a core-shell structure in an aqueous medium without the need for self-assembly, so that a drug delivery system capable of responsively releasing a drug active ingredient can be prepared for the treatment of tumor diseases.

In the present application, the monomer represented by formula (I-3) is a branched amino acid monomer, in which two NCA functional groups are connected through a platinum atom as a bridge. This monomer can form the non-linear backbone through ring-opening polymerization, providing branch points in the non-linear backbone.

In the present application, the monomer represented by formula (II-3) and the monomer represented by formula (IV-3) are both non-branched amino acid monomers.

The monomer represented by formula (II-3) has an NCA functional group at one end and a second drug unit D_{T} at the other end. The monomer represented by formula (II-3) can form a part of the poly(amino acid) chain through the ring-opening polymerization, but cannot provide branch points in the non-linear backbone.

The monomer represented by formula (IV-3) is an NCA-functionalized amino acid, including an NCA functional group and not including other reactive groups participating in the ring-opening polymerization. That is, only the NCA functional group participates in the ring-opening polymerization. The monomer represented by formula (IV-3) can form a part of the poly(amino acid) chain, but cannot provide branch points in the non-linear backbone.

It can be understood that, upon the monomer represented by formula (1-3), addition of the monomer represented by formula (II-3) and/or the monomer represented by formula (IV-3) can space the branch points of the monomers represented by formula (1-3) in the poly(amino acid) chain. This allows for the adjustment of the distribution density of the branch points in the poly(amino acid) chain, and the adjustment of the overall branching degree or cross-linking degree of the drug-loaded monomolecular nano-polymer.

The monomer represented by formula (III-3) can act as a terminating agent. The more the terminating agent, the shorter the length of the poly(amino acid) chain and the smaller the size of the drug-loaded monomolecular nano-polymer. Thus, the molecular size and the drug-loading amount of the drug-loaded monomolecular nano-polymer can be adjusted by controlling the amount of the monomer represented by formula (III-3).

In some embodiments, the molar percentage of the monomer represented by formula (I-3) relative to all amino acid monomers, specifically all NCA-functionalized amino acid monomers, can be 15% to 100%, preferably 15% to 90%, or preferably 15% to 80%, or preferably 15% to 60%, or preferably 15% to 50%, or preferably 15% to 40%, or preferably 15% to 30%, or preferably 20% to 80%, or preferably 20% to 60%, or preferably 20% to 50%, or preferably 20% to 40%, or preferably 20% to 30%. The molar percentage of the monomer represented by formula (I-3) relative to all amino acid monomers can be 15%, 16%, 17%, 18%, 19%, 20%, 22%, 24%, 25%, 26%, 28%, 30%, 35%, 40%, 45%, 50%, 55%, 60%, 65%, 70%, 75%, 80%, 85%, 90%, 95%, 96%, 97%, 98%, 99%, or 100%, or in an interval defined by any two of the above values.

In some embodiments, the molar ratio of the monomer represented by formula (II-3) to the monomer represented by formula (I-3) can be equal to the ratio of the number of the second drug units to the number of the platinum-based drug units. The molar ratio of the monomer represented by formula (II-3) to the monomer represented by formula (I-3) can be in a range from 0:1 to 10:1, preferably 0:1 to 5:1, or preferably 0:1 to 3:1, or preferably 0:1 to 1:1, or preferably 0.5:1 to 10:1, or preferably 0.5:1 to 5:1, or preferably 0.5:1 to 3:1, or preferably 1:1 to 5:1, or preferably 1:1 to 3:1, or preferably 2:1 to 3:1. The molar ratio of the monomer represented by formula (II-3) to the monomer represented by formula (I-3) can be 0.1:1, 0.2:1, 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.8:1, 2:1, 2.5:1, 2.6:1, 2.8:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1, or in an interval defined by any two of the above values.

In some embodiments, the molar ratio of the monomer represented by formula (IV-3) to the monomer represented by formula (I-3) can be equal to the ratio of the number of the hydrophilic polymer chains to the number of the platinum-based drug units. The molar ratio of the monomer represented by formula (IV-3) to the monomer represented by formula (I-3) can be in a range from 1:2 to 1:100, preferably 1:10 to 1:60, or preferably 1:15 to 1:45, or preferably 1:15 to 1:25. The molar ratio of the monomer represented by formula (IV-3) to the monomer represented by formula (I-3) can be 1:2, 1:3, 1:4, 1:5, 1:6, 1:7, 1:8, 1:9, 1:10, 1:11, 1:12, 1:13, 1:14, 1:15, 1:16, 1:18, 1:20, 1:22, 1:24, 1:25, 1:26, 1:28, 1:30, 1:35, 1:40, 1:45, 1:55, 1:60, 1:65, 1:70, 1:75, 1:80, 1:85, 1:90, 1:95, or 1:100, or in an interval defined by any two of the above values.

According to the present application, a dual-drug monomolecular nano-polymer prodrug includes a poly(amino acid) connected with a hydrophilic polymer, wherein a prodrug moiety of a platinum-based drug active ingredient and a prodrug moiety of an anti-tumor drug active ingredient are respectively bonded to α-carbons of repeating units of the poly(amino acid). Preferably, the molecule of the anti-tumor drug active ingredient includes a free hydroxyl group, a free amino group, or a combination thereof.

In some embodiments of the dual-drug monomolecular nano-polymer prodrug, the dual-drug monomolecular nano-polymer prodrug includes a random-copolymerized amino acid backbone connected with the hydrophilic polymer.

In some embodiments of the dual-drug monomolecular nano-polymer prodrug, the hydrophilic polymer is selected from the group consisting of poly(alkylene glycol) (such as polyethylene glycol (PEG), poly(propylene glycol) (PPG), a copolymer of ethylene glycol and propylene glycol, etc.), poly(ethoxylated polyol), poly(alkenol), poly(vinylpyrrolidone), poly(hydroxyalkyl methacrylamide), poly (hydroxyalkyl methacrylate), polysaccharide, poly(α-hydroxyacid), poly(vinyl alcohol), polyphosphazene, polyoxazoline (POZ), poly(N-acryloylmorpholine), and any combination thereof.

In a preferred embodiment of the dual-drug monomolecular nano-polymer prodrug, the hydrophilic polymer is polyethylene glycol (PEG), preferably polyethylene glycol terminated by methoxy at one end. The molecular weight of the hydrophilic polymer is not particularly limited. For example, PEG50-5000, PEG50-6000, PEG50-7000, PEG50-8000, PEG500-2000, PEG500-4000, PEG500-6000, PEG500-8000, PEG500-10000, PEG500-20000, PEG1000-20000, PEG1000-50000, or PEG1000-80000 in the unit of Da can be used in the present application. For example, PEG500-4000 means PEG with a molecular weight in a range from 500 Da to 4000 Da.

In some embodiments of the drug-loaded monomolecular nano-polymer and the dual-drug monomolecular nano-polymer prodrug, the platinum-based drug is any one of cisplatin, carboplatin, nedaplatin, oxaliplatin, or lobaplatin. The chemical structures, preparation methods, and pharmacological effects of these platinum-based drugs are all known in the art. For example, cisplatin is a cell-cycle non-specific anti-cancer drug, with the following structure:

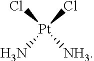

In a preferred embodiment of the drug-loaded monomolecular nano-polymer and the dual-drug monomolecular nano-polymer prodrug, the platinum-based drug active ingredient is cisplatin.

In some embodiments of the drug-loaded monomolecular nano-polymer and the dual-drug monomolecular nano-polymer prodrug, the anti-tumor drug active ingredient whose molecule includes a free hydroxyl group, a free amino group, or a combination thereof is selected from the following compounds: a camptothecin-based compound; a tumor immune activator, such as resiquimod (R-848), telratolimod, etc.; paclitaxel (PTX), epirubicin, docetaxel, pemetrexed, methylauristatin E, gemcitabine, dexamethasone; a protein kinase inhibitor, such as sorafenib, dasatinib, etc. The camptothecin-based compound can be camptothecin, derivatives or analogs thereof, such as irinotecan, topotecan, rubitecan, chimmitecan, 9-aminocamptothecin, 9-nitrocamptothecin, 7-ethyl-10-hydroxycamptothecin (SN38), etc.

The chemical structures, preparation methods, and pharmacological effects of these anti-tumor drug active ingredients are all known in the art. For example, camptothecin is a DNA topoisomerase I inhibitor, with the following structure:

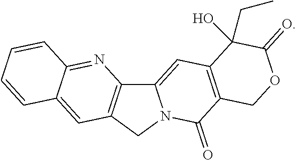 [SMILES: CCC1(O)C(=O)OCc2c1cc1n(c2=O)Cc2cc3ccccc3nc2-1]

Resiquimod (R-848) is an immune response modifier that can enhance immunity against tumor, with the following structure:

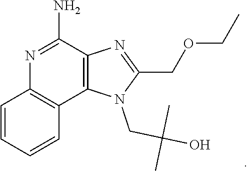 [SMILES: CCOCc1nc2c(N)nc3ccccc3c2n1CC(C)(C)O]

Paclitaxel (PTX), as an alkaloid extracted from the plants of Tams, is a cell cycle-specific anti-tumor drug, which promotes tubulin polymerization and inhibits tubulin depolymerization to maintain the stability of tubulin, thereby inhibiting cell mitosis. The chemical name of paclitaxel is 5β, 20-epoxy-1,2α,4,7β,10β,13α-hexahydroxytax-11-en-9-one-4,10-diacetate-2-benzoate-13[(2′R,3'S)—N-benzoyl-3-phenylis oserine ester], with the following structure:

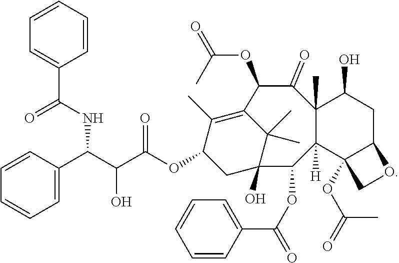 [SMILES: CC(=O)O[C@H]1C(=O)[C@@]2(C)[C@H]([C@H](OC(=O)c3ccccc3)[C@]3(O)C[C@H](OC(=O)C(O)[C@@H](NC(=O)c4ccccc4)c4ccccc4)C(C)=C1C3(C)C)[C@]1(OC(C)=O)CO[C@@H]1C[C@@H]2O]

In preferred embodiments of the drug-loaded monomolecular nano-polymer and the dual-drug monomolecular nano-polymer prodrug, the anti-tumor drug active ingredient whose molecule includes a free hydroxyl group, a free amino group, or a combination thereof is camptothecin.

In some embodiments of the drug-loaded monomolecular nano-polymer and the dual-drug monomolecular nano-polymer prodrug, the dual-drug monomolecular nano-polymer prodrug has the following structure:

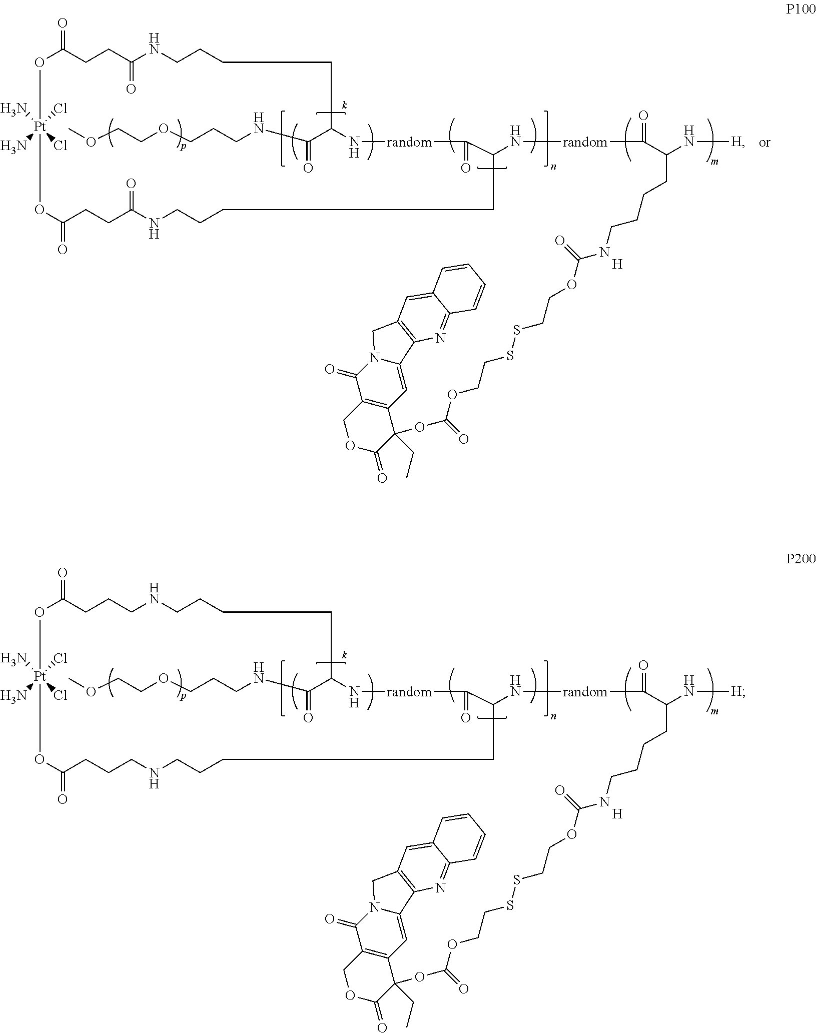

wherein p can be in a range from 1 to 500, m can be in a range from 0 to 100, n can be in a range from 1 to 100, k can be in a range from 1 to 1000, but theses ranges are not limiting; p represents the degree of polymerization of the polyethylene glycol segment, or the definition of p can be found in other parts of the present application.

In the above general formula, m can represent the number of the second drug units in one molecule; n can represent the number of platinum atoms (in L_{Pt}) in one molecule; k can represent the number of mPEG segments in one molecule.

In some embodiments, the number m of the second drug units in each molecule can be in a range from 0 to 5000, further can be in a range from 0 to 4000, further can be in a range from 0 to 2000, further can be in a range from 0 to 1500, further can be in a range from 0 to 1000, further can be in a range from 0 to 500, further can be in a range from 10 to 2000, further can be in a range from 10 to 1500, further can be in a range from 10 to 1000, further can be in a range from 10 to 500, further can be in a range from 20 to 2000, further can be in a range from 20 to 1500, further can be in a range from 20 to 1000, further can be in a range from 20 to 500, further can be in a range from 40 to 2000, further can be in a range from 40 to 1500, further can be in a range from 40 to 1000, further can be in a range from 40 to 500, further can be in a range from 50 to 2000, further can be in a range from 50 to 1500, further can be in a range from 50 to 1000, further can be in a range from 50 to 500, further can be in a range from 80 to 2000, further can be in a range from 80 to 1500, further can be in a range from 80 to 1000, further can be in a range from 80 to 500, further can be in a range from 80 to 2000, further can be in a range from 80 to 1500, further can be in a range from 80 to 1000, further can be in a range from 80 to 500, further can be in a range from 100 to 2000, further can be in a range from 100 to 1500, further can be in a range from 100 to 1000, further can be in a range from 100 to 500, further can be in a range from 500 to 1500, further can be in a range from 600 to 1500, further can be in a range from 800 to 1500, further can be in a range from 800 to 1200. In each molecule, the number m of the second drug units can be 0, 1, 2, 3, 4, 5, 6, 7, 8, 9, 10, 11, 12, 13, 14, 15, 16, 18, 20, 25, 30, 35, 40, 45, 50, 60, 70, 80, 90, 100, 120, 150, 200, 250, 300, 350, 400, 450, 500, 550, 600, 650, 700, 750, 800, 900, 1000, 1100, 1200, 1300, 1400, 1500, 1600, 1800, 2000, 2500, or 3000, or in an interval defined by any two of the above values.

It should be noted that the amino acid unit in the general formula is lysine. The lysine units in the general formula form a non-linear backbone through the Pt-containing linking groups. The non-linear backbone can include a plurality of polylysine chains. The amino acid units corresponding to k and the amino acid units corresponding to n can belong to different polylysine chains. Different polylysine chains can each include lysine units corresponding to m. The C-terminus of each polylysine chain can be terminated by a polyethylene glycol segment. The “random” in the above general formula represents that the amino acid units are randomly polymerized.

In specific embodiments of the drug-loaded monomolecular nano-polymer and the dual-drug monomolecular nano-polymer prodrug, the dual-drug monomolecular nano-polymer prodrug has the following structure:

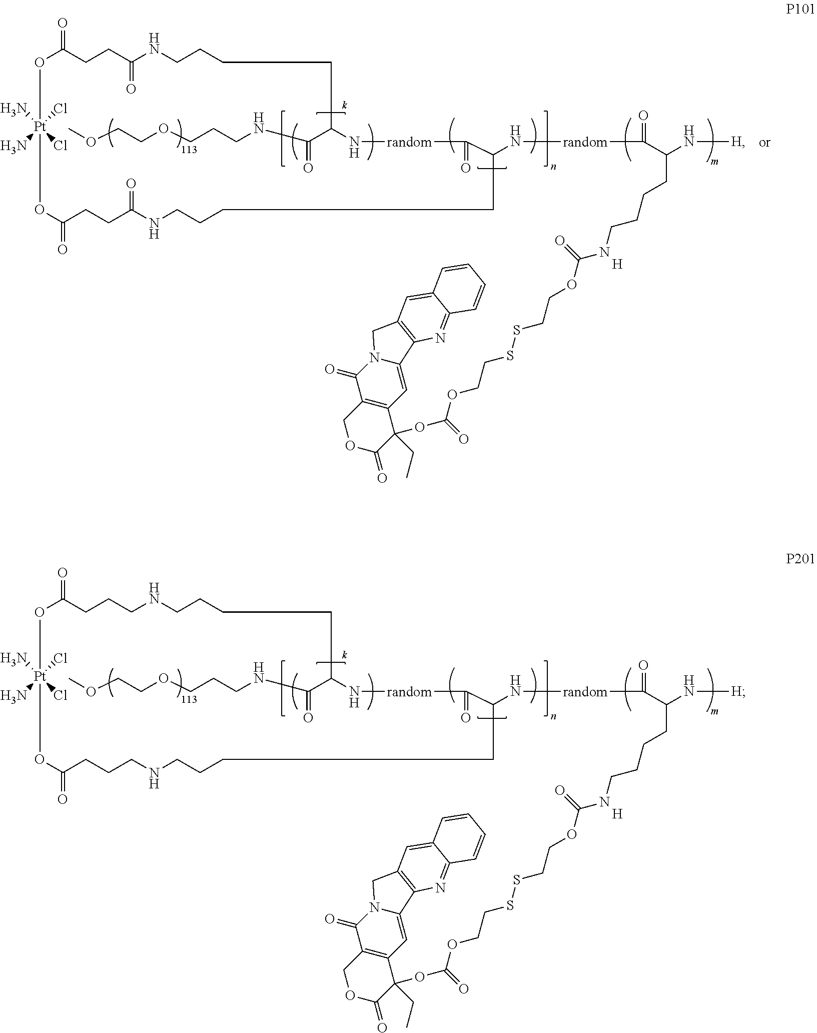

wherein m can be in a range from 0 to 100, n can be in a range from 1 to 100, k can be in a range from 1 to 1000, but these ranges are not limiting.

According to the present application, a drug delivery system includes a dual-drug monomolecular nano-polymer micelle. The polymer micelle includes a poly(amino acid) connected with a hydrophilic polymer. A prodrug moiety of a platinum-based drug active ingredient and a prodrug moiety of an anti-tumor drug active ingredient are respectively bonded to α-carbons of repeating units of the poly(amino acid). Preferably, the molecule of the anti-tumor drug active ingredient includes a free hydroxyl group, a free amino group, or a combination thereof.

In some embodiments of the drug delivery system, the dual-drug monomolecular nano-polymer micelle includes a random amino acid copolymer backbone connected with the hydrophilic polymer.

In some embodiments of the drug delivery system, the hydrophilic polymer is selected from the group consisting of poly(alkylene glycol) (such as, polyethylene glycol (PEG), poly(propylene glycol) (PPG), a copolymer of ethylene glycol and propylene glycol, etc.), poly(ethoxylated polyol), poly(alkenol), poly(vinylpyrrolidone), poly(hydroxyalkyl methacrylamide), poly (hydroxyalkyl methacrylate), polysaccharide, poly(α-hydroxyacid), poly(vinyl alcohol), polyphosphazene, polyoxazoline (POZ), poly(N-acryloylmorpholine), poly(2-methacryloyloxyethyl phosphorylcholine) (PMPC), and any combination thereof.

In preferred embodiments of the drug delivery system, the hydrophilic polymer is polyethylene glycol (PEG), preferably methoxy-terminated polyethylene glycol.

In some embodiments of the drug delivery system, the platinum-based drug is selected from cisplatin, carboplatin, nedaplatin, oxaliplatin, or lobaplatin.

In preferred embodiments of the drug delivery system, the platinum-based drug active ingredient is cisplatin.

In some embodiments of the drug delivery system, the anti-tumor drug active ingredient whose molecule includes a free hydroxyl group, a free amino group, or a combination thereof is a camptothecin-based compound, resiquimod (R-848), or paclitaxel (PTX). The camptothecin-based compound can be camptothecin, derivatives or analogs thereof, such as irinotecan, topotecan, rubitecan, chimmitecan, 9-aminocamptothecin, 9-nitrocamptothecin, 7-ethyl-10-hydroxycamptothecin (SN38), etc.

In preferred embodiments of the drug delivery system, the anti-tumor drug active ingredient whose molecule includes a free hydroxyl group, a free amino group, or a combination thereof is camptothecin.

In some embodiments of the drug delivery system, the dual-drug monomolecular nano-polymer prodrug has the structure shown in P100 or P200.

In some embodiments of the drug delivery system, the dual-drug monomolecular nano-polymer prodrug has the following structure:

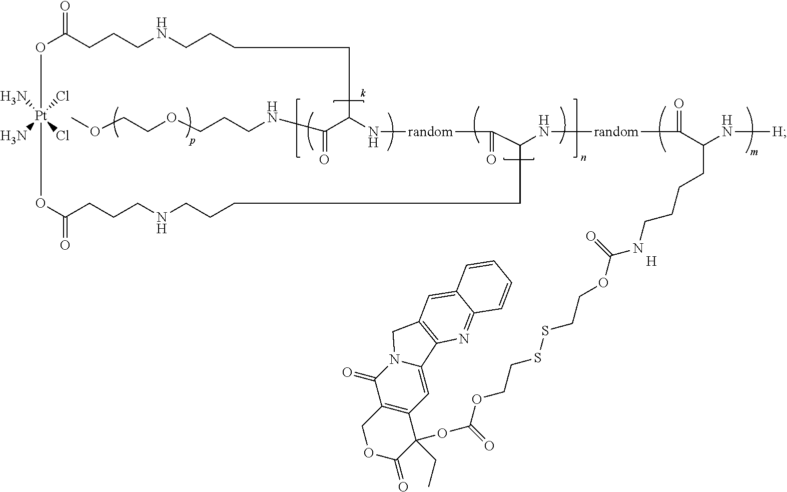

wherein p can be in a range from 1 to 500, m can be in a range from 0 to 100, n can be in a range from 1 to 100, k can be in a range from 1 to 1000, but theses ranges are not limiting.

In some specific embodiments of the drug delivery system, the dual-drug monomolecular nano-polymer prodrug has the structure shown in P101 or P201.

In some specific embodiments of the drug delivery system, the dual-drug monomolecular nano-polymer prodrug has the following structure:

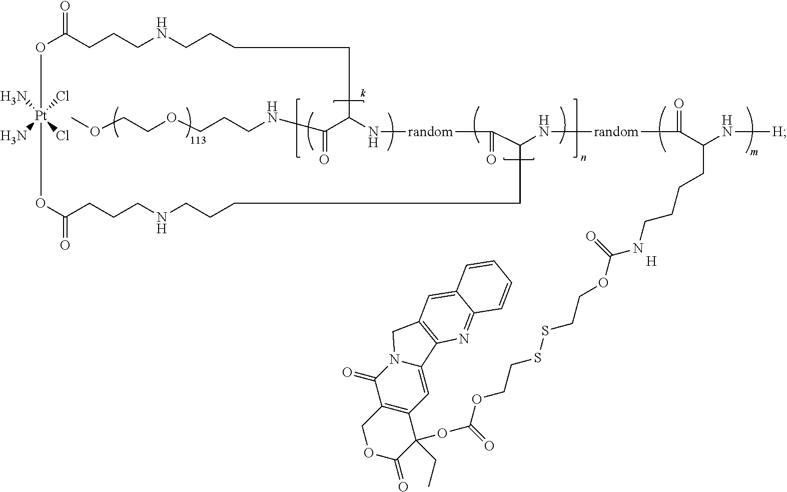

wherein m can be in a range from 0 to 100, n can be in a range from 1 to 100, k can be in a range from 1 to 1000, but theses ranges are not limiting.

The dual-drug monomolecular nano-polymer prodrug provided in the present application can be prepared by a method including the following steps:
(1) synthesizing a single-NCA monomer corresponding to an anti-tumor drug active ingredient under an appropriate reaction condition; preferably, the molecular structure of the anti-tumor drug active ingredient includes a free hydroxyl group or a free amino group;
(2) synthesizing a bis-NCA monomer corresponding to an platinum-based drug active ingredient under an appropriate reaction condition;
(3) reacting the single-NCA monomer in step (1) and the bis-NCA monomer in step (2) with a hydrophilic polymer having a terminal amino group under an appropriate reaction condition, thereby obtaining the dual-drug monomolecular nano-polymer prodrug; and
(4) separating and purifying the dual-drug monomolecular nano-polymer prodrug.

In some preferred embodiments, the single-NCA monomer corresponding to the anti-tumor drug active ingredient including the free hydroxyl group or the free amino group is synthesized according to the scheme below.

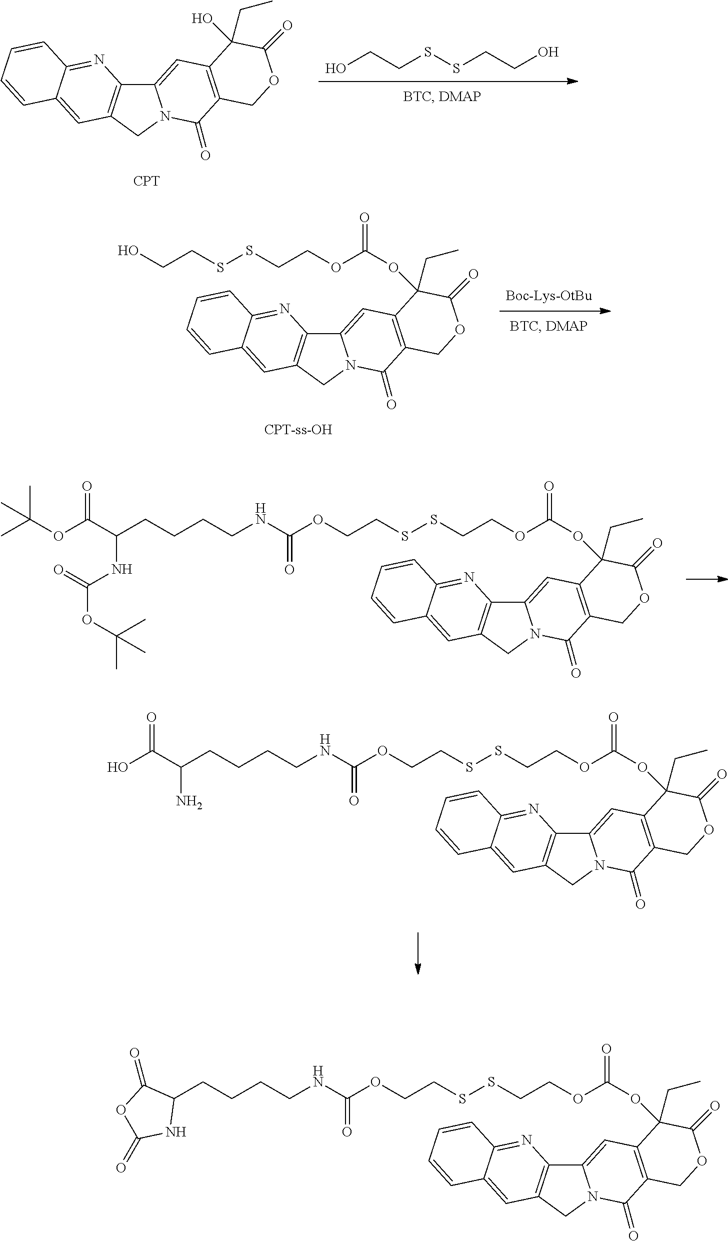

Boc-Lyc-OtBu can be synthesized according to the scheme below.

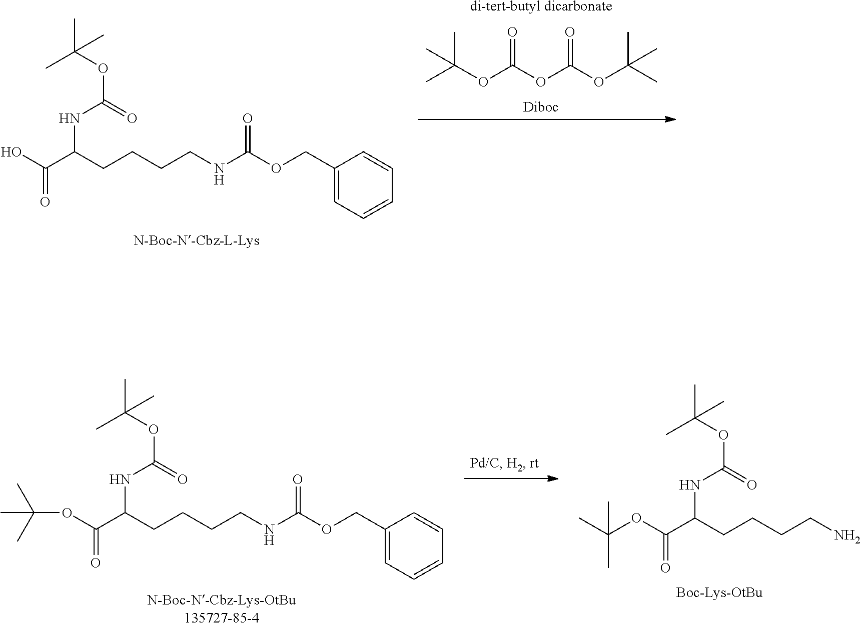

In preferred embodiments, the single-NCA monomer corresponding to the anti-tumor drug active ingredient including the free hydroxyl group or the free amino group is synthesized according to the scheme below.

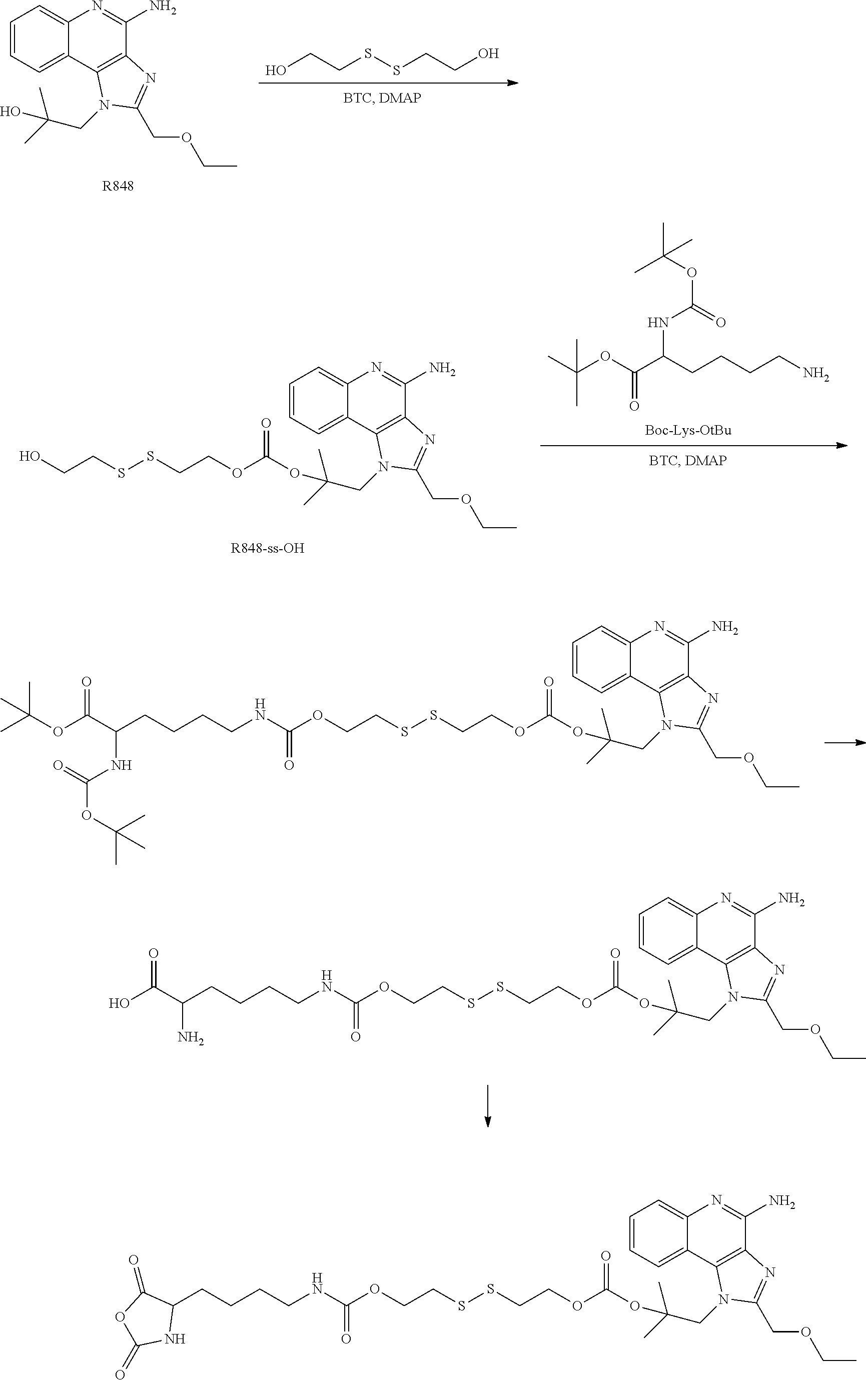

In some preferred embodiments, the bis-NCA monomer corresponding to the platinum-based drug active ingredient is synthesized according to the scheme below.

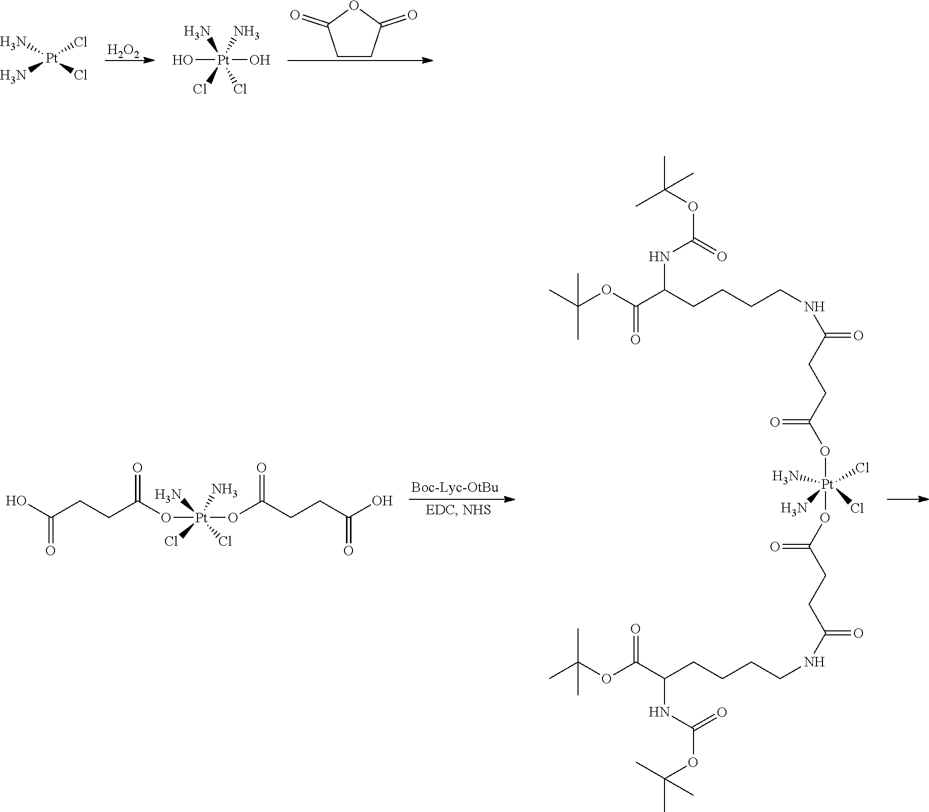

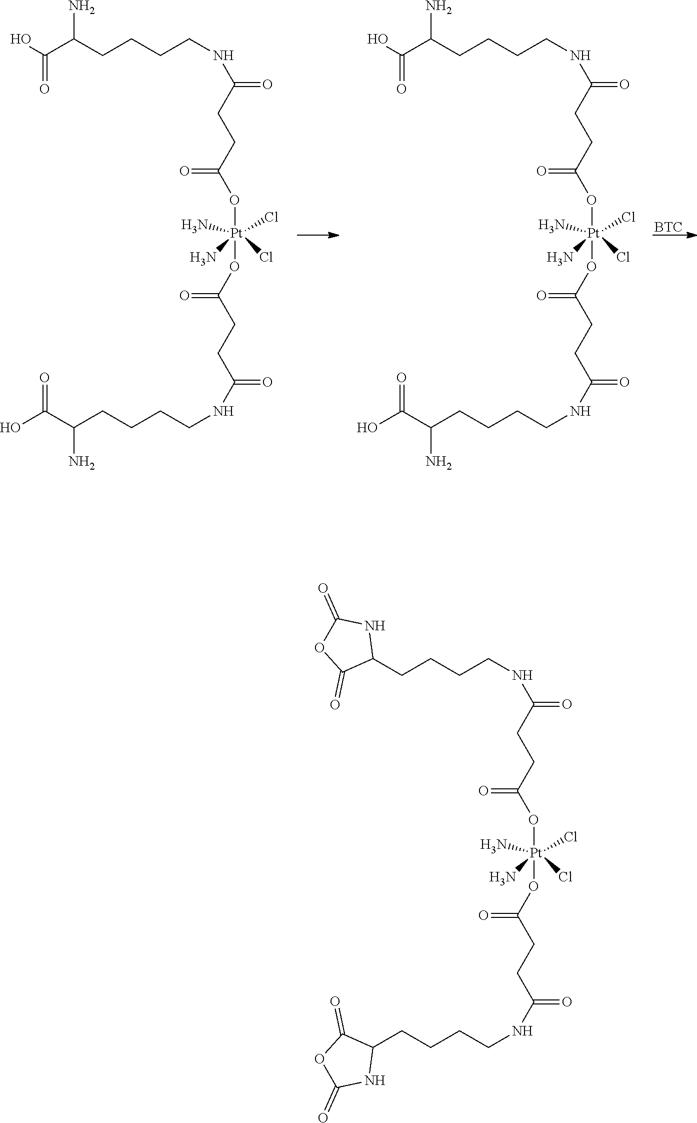

In some preferred embodiments, the duel-drug monomolecular nano-polymer prodrug is synthesized to the “one-pot method” below.

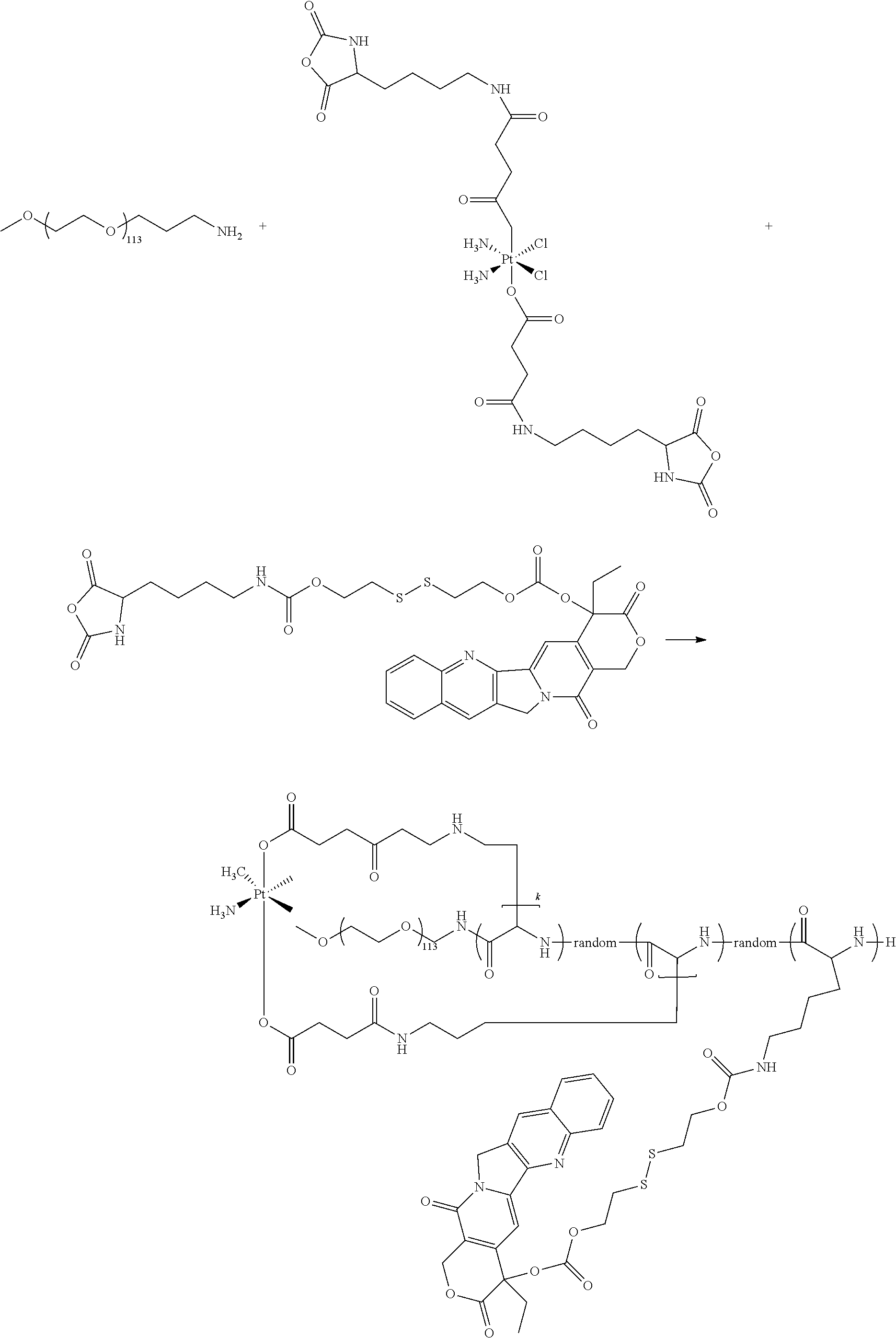

p can be in a range from 1 to 500, m can be in a range from 0 to 100, n can be in a range from 1 to 100, k can be in a range from 1 to 1000, but theses ranges are not limiting.

In some embodiments, the duel-drug monomolecular nano-polymer prodrug of the present application is synthesized by a method including the following steps.

A hydrophilic polymer (such as polyethylene glycol) including a terminal amino group is dissolved in an organic solvent such as benzene, frozen, and then dried under vacuum in a cold trap.

In a glove box, the dried hydrophilic polymer is dissolved in an anhydrous organic solvent such as DMF. The signal-NCA monomer corresponding to the anti-tumor drug active ingredient including the free hydroxyl group or the free amino group and the bis-NCA monomer corresponding to the platinum-based drug active ingredient are dissolved in a same anhydrous organic solvent. The resulting solution is slowly added to the reaction system dropwise. The reaction tube is sealed, taken out from the glove box, and continuously stirred in an oil bath for a sufficient time for reaction.

The reaction product is slowly added dropwise into anhydrous diethyl ether to obtain a white precipitate, and the supernatant is discarded, thereby obtaining a purified product.

The purified product is vacuum dried to obtain a solid, which is then dissolved in a suitable solvent such as DMSO, and the resulting solution is introduced into a dialysis bag with MWCO of 100 kDa, dialyzed in ultrapure water for several days (during which water is changed several times). After freeze-drying, the final product, the dual-drug monomolecular nano-polymer prodrug, is collected.

In a specific embodiment, the dual-drug monomolecular nano-polymer prodrug of the present application is synthesized by the method including the following steps.

Methoxy-terminated polyethylene glycol including a free amino group is dissolved in benzene, frozen, and then dried under vacuum in a cold trap.

In a glove box, the dried methoxy-terminated polyethylene glycol including the free amino group is dissolved in DMF. The single-NCA monomer corresponding to the anti-tumor drug active ingredient including the free hydroxyl group or the free amino group and the bis-NCA monomer corresponding to the platinum-based drug active ingredient are dissolved in DMF. The resulting solution is slowly added dropwise to the reaction system. The reaction tube is sealed, taken out from the glove box, and continuously stirred in an oil bath for a sufficient time for reaction.

The reaction product is added dropwise into anhydrous diethyl ether to obtain a white precipitate, and the supernatant is discarded, thereby obtaining a purified product.

The purified product is vacuum dried to obtain a solid, which is then dissolved in DMSO, and the resulting solution is introduced into a dialysis bag with MWCO of 100 kDa, dialyzed in ultrapure water for two days (during which water is changed five times). After freeze-drying, the final product, the dual-drug monomolecular nano-polymer prodrug, is collected.

In another aspect of the present application, a drug-loaded monomolecular nano-polymer micelle is provided. The drug-loaded monomolecular nano-polymer micelle is made of any one of the drug-loaded monomolecular nano-polymer as described above, the drug-loaded monomolecular nano-polymer prepared by the method as described above, the dual-drug monomolecular nano-polymer prodrug as described above, or the dual-drug monomolecular nano-polymer prodrug prepared by the method as described above. The drug-loaded monomolecular nano-polymer micelle has a core-shell structure. The shell is a hydrophilic layer made of the hydrophilic polymer chain(s), and the drug unit(s) is encapsulated in the core.

The drug-loaded monomolecular nano-polymer can be controlled to have a branched three-dimensional structure with an appropriate cross-linking degree by adjusting the distribution density of L_{Pt}. In further combination with the design of the hydrophilic polymer chain at the end of the poly(amino acid) chain, the drug-loaded monomolecular nano-polymer can form a drug-loaded monomolecular nano-polymer micelle with a core-shell structure in an aqueous medium without the need for self-assembly. The hydrophilic polymer chain is distributed as the shell, and the drug component is encapsulated within the core.

In another aspect of the present application, a drug delivery system is provided. The drug delivery system includes the drug-loaded monomolecular nano-polymer micelle. The drug-loaded monomolecular nano-polymer micelle includes the drug-loaded monomolecular nano-polymer as described above, or includes the drug-loaded monomolecular nano-polymer prepared by the method as described above. Preferably, the hydrophilic polymer chain is located in the shell of the drug-loaded monomolecular nano-polymer micelle, and the platinum-based drug unit and the second drug unit are both located in the core of the drug-loaded monomolecular nano-polymer micelle.

In the present application, unless otherwise specified, the particle size of the drug-loaded monomolecular nano-polymer micelle is tested at a temperature of 20° C. to 30° C., such as 25° C.

The size and morphology of the drug-loaded monomolecular nano-polymer micelle can be characterized by using the test methods in following embodiments.

In the present application, only when specified, the particle size of the drug-loaded monomolecular nano-polymer micelle refers to an average diameter or an average particle size.

In the present application, when the size of the drug-loaded monomolecular nano-polymer micelle is measured, the test condition “in water” can be “in pure water” or “in an aqueous solution”. Examples of the aqueous solution include a buffer solution such as a PBS solution, a physiological simulation solution, etc.

In some embodiments, the particle size of the drug-loaded monomolecular nano-polymer micelle is in a range from 10 to 120 nm, preferably 10 to 110 nm, or preferably 10 to 100 nm, or preferably 10 to 80 nm, or preferably 10 to 50 nm, or preferably 10 to 40 nm, or preferably 10 to 30 nm, or preferably 15 to 120 nm, or preferably 15 to 110 nm, or preferably 15 to 100 nm, or preferably 15 to 80 nm, or preferably 15 to 50 nm, or preferably 15 to 40 nm, or preferably 15 to 30 nm, or preferably 20 to 120 nm, or preferably 20 to 110 nm, or preferably 20 to 100 nm, or preferably 20 to 80 nm, or preferably 20 to 70 nm, or preferably 20 to 50 nm, or preferably 20 to 40 nm, or preferably 25 to 120 nm, or preferably 25 to 110 nm, or preferably 25 to 100 nm, or preferably 25 to 80 nm, or preferably 25 to 50 nm, or preferably 25 to 40 nm, or preferably 25 to 35 nm, or preferably 30 to 35 nm. The particle size of the drug-loaded monomolecular nano-polymer micelle can be 15 nm, 16 nm, 18 nm, 20 nm, 25 nm, 30 nm, 35 nm, 40 nm, 45 nm, 50 nm, 55 nm, 60 nm, 65 nm, 70 nm, 80 nm, 90 nm, 100 nm, 110 nm, or 120 nm, or in an interval defined by any two of the above values. The temperature for testing the particle size can be in range from 20° C. to 30° C., further 25° C. The particle size can be obtained by dynamic light scattering in water, or can be obtained by transmission electron microscopy.

In some embodiments, the particle size of the drug-loaded monomolecular nano-polymer micelle obtained by transmission electron microscopy (referring to Example 4) is equal to or less than 120 nm, further equal to or less than 100 nm, further equal to or less than 90 nm, further equal to or less than 8 nm, further equal to or less than 70 nm, further equal to or less than 60 nm, further equal to or less than 50 nm.

In some embodiments, the average diameter of the drug-loaded monomolecular nano-polymer micelles is in a range from 15 to 50 nm, or can be in a range from 15 to 40 nm, or can be in a range from 20 to 40 nm, or can be in a range from 25 to 35 nm. The temperature for testing the average diameter can be in a range from 20° C. to 30° C., further 25° C. The average diameter can be obtained by dynamic light scattering in water, or can be obtained by transmission electron microscopy.

In some embodiments, the core radius of the drug-loaded monomolecular nano-polymer micelle is in a range from 5 to 50 nm, or can be in a range from 5 to 45 nm, or can be in a range from 5 to 40 nm, or can be in a range from 5 to 35 nm, or can be in a range from 5 to 30 nm, or can be in a range from 5 to 25 nm, or can be in a range from 5 to 20 nm, further can be in a range from 5 to 15 nm, or can be in a range from 5 to 10 nm, or can be in a range from 10 to 50 nm, or can be in a range from 10 to 45 nm, or can be in a range from 10 to 40 nm, or can be in a range from 10 to 35 nm, or can be in a range from 10 to 30 nm, or can be in a range from 10 to 25 nm, or can be in a range from 10 to 20 nm, or can be in a range from 6 to 8 nm. The core radius of the drug-loaded monomolecular nano-polymer micelle can be 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 22 nm, 24 nm, 25 nm, 30 nm, 35 nm, 40 nm, 45 nm, or 50 nm, or in an interval defined by any two of the above values. The temperature for testing the core radius can be in a range from 20° C. to 30° C., further 25° C. The core radius can be tested in water, further can be obtained by small angle X-ray scattering (SAXS).

In some embodiments, the thickness of the shell of the drug-loaded monomolecular nano-polymer micelle in water at 25° C. is in a range from 5 to 40 nm, or can be in a range from 5 to 35 nm, or can be in a range from 5 to 30 nm, or can be in a range from 5 to 25 nm, or can be in a range from 5 to 20 nm, or can be in a range from 5 to 15 nm, or can be in a range from 10 to 40 nm, or can be in a range from 10 to 35 nm, or can be in a range from 10 to 30 nm, or can be in a range from 10 to 25 nm, or can be in a range from 10 to 20 nm, or can be in a range from 8 to 12 nm. The thickness of the shell of the drug-loaded monomolecular nano-polymer micelle in water at 25° C. can be 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 22 nm, 24 nm, 25 nm, 30 nm, 35 nm, or 40 nm, or in an interval defined by any two of the above values. The thickness of the shell can be tested in water, further can be obtained by small angle X-ray scattering (SAXS).

In another aspect of the present application, a drug delivery system is provided. The drug delivery system includes a dual-drug monomolecular nano-polymer micelle. The dual-drug monomolecular nano-polymer micelle includes a poly(amino acid) connected with a hydrophilic polymer. A prodrug moiety of a platinum-based drug active ingredient and a prodrug moiety of an anti-tumor drug active ingredient are respectively bonded to α-carbons of a repeating units of the poly(amino acid). Preferably, the molecule of the anti-tumor drug active ingredient includes a free hydroxyl group, a free amino group, or a combination thereof.

In some embodiments, the hydrophilic polymer is selected from polyethylene glycol, poly(propylene glycol), a copolymer of ethylene glycol and propylene glycol, poly(ethoxylated polyol), poly(alkenol), poly(vinylpyrrolidone), poly(hydroxyalkyl methacrylamide), poly(hydroxyalkyl methacrylate), polysaccharide, poly(α-hydroxyacid), poly(vinyl alcohol), polyphosphazene, polyoxazoline, poly(N-acryloylmorpholine), and any combination thereof.

In some embodiments, the platinum-based drug includes one or more of cisplatin, carboplatin, nedaplatin, oxaliplatin, or lobaplatin.

In some embodiments, the anti-tumor drug active ingredient whose molecule includes a free hydroxyl group, a free amino group or a combination thereof is one or more of a camptothecin-based compound, resiquimod, or paclitaxel. Preferably, the camptothecin-based compounds can be camptothecin, derivatives or analogs thereof. More preferably, the camptothecin-based compounds include irinotecan, topotecan, rubitecan, chimmitecan, 9-aminocamptothecin, 9-nitrocamptothecin, or 7-ethyl-10-hydroxycamptothecin.

Therefore, the drug-loaded monomolecular nano-polymer micelle or the dual-drug active ingredient nano-polymer prodrug micelle can be easily prepared by those skilled in the art according to the above methods and the methods described in the following examples.

In another aspect of the present application, use of a bis-NCA monomer corresponding to a platinum-based drug active ingredient and a single-NCA monomer corresponding to an anti-tumor drug active ingredient in the preparation of a monomolecular nano-polymer prodrug or a drug delivery system is provided. Preferably, the molecular structure of the anti-tumor drug active ingredient includes a free hydroxyl group or a free amino group.

In another object of the present application, use of the drug-loaded monomolecular nano-polymer described above as a prodrug is provided. The drug-loaded monomolecular nano-polymer can enter the interior of a tumor cell and release the drug active ingredient in response to the intracellular microenvironment of the tumor cell, thereby producing cytotoxicity and inhibiting the growth of the tumor cell.

In some embodiments, the drug release mechanisms are as follows: (a) tetravalent platinum is reduced in the highly reducing intracellular microenvironment to shed the ligands connected to the poly(amino acid), leading to selectively release of the divalent platinum active species in the cell; and (b) in the highly reducing intracellular microenvironment, especially in reaction with the high concentration of glutathione in the cell, the disulfide bond cleaves to form free thiol groups, which in turn attack the carbonate bond or the carbamate bond connected to the anti-tumor drug, leading to selectively release of the anti-tumor drug active ingredient in the cell.

In an example of the structural unit of formula (II) as shown below, with a residue of camptothecin (CPT) as the second drug unit, and a disulfide bond as the responsive linking group L_{R}, the drug active ingredient CPT is released according to the following molecular mechanism.

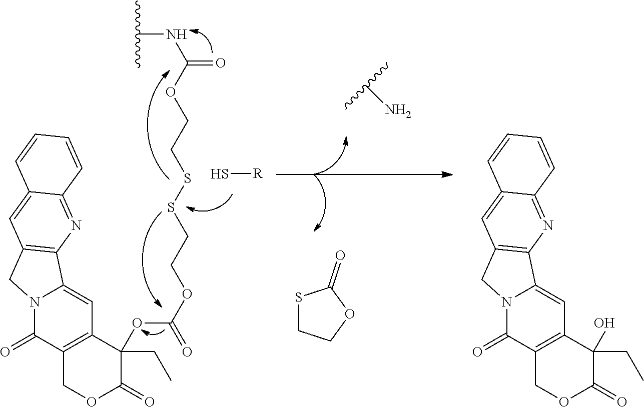

In an example of the structural unit of formula (I) as shown below, with a residue of cisplatin as the platinum-based drug unit, the active Pt(II) drug is released from the tetravalent platinum according to the following molecular mechanism;

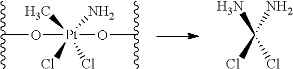

and specifically according to the following molecular mechanism:

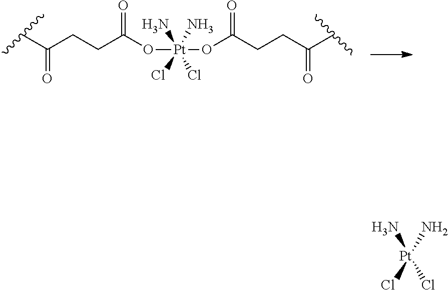

In another aspect of the present application, there is provided use of the drug-loaded monomolecular nano-polymer as described above, use of the drug-loaded monomolecular nano-polymer prepared by the method as described above, use of the dual-drug monomolecular nano-polymer prodrug as described above, use of the dual-drug monomolecular nano-polymer prodrug prepared by the method as described above, use of the drug-loaded monomolecular nano-polymer micelle as described above, or use of the drug delivery system as described above in the manufacture of a medicament for treating tumor diseases.

The tumor diseases can include but are not limited to lung cancer, gastric cancer, bladder cancer, ovarian cancer, testicular cancer, endometrial cancer, bone cancer, sarcomas, cervical cancer, esophageal cancer, liver cancer, colorectal cancer, head and neck cancers, chorionic epithelioma, malignant hydatidiform mole, non-Hodgkin lymphoma, acute and chronic myelogenous leukemia, etc., and can also include but are not limited to lung cancer, esophageal cancer, and head and neck tumors.

Based on the above, those skilled in the art can easily further prepare a desired formulation of the drug-loaded monomolecular nano-polymer micelles or the nano-polymer prodrug micelles of the present application according to the methods described in the present application and the techniques known in the art.

The drug-loaded monomolecular nano-polymer or the dual-drug active ingredient nano-polymer prodrug of the present application can be used in the treatment of lung cancer, gastric cancer, bladder cancer, ovarian cancer, testicular cancer, endometrial cancer, bone cancer, sarcomas, cervical cancer, esophageal cancer, liver cancer, colorectal cancer, head and neck cancers, chorionic epithelioma, malignant hydatidiform mole, non-Hodgkin lymphoma, acute and chronic myelogenous leukemia, etc. The nano-formulation of the present application can also be used to increase the sensitivity of tumor cells to radiotherapy, with concurrent administration during radiotherapy enhancing the control of local progression of lung cancer, esophageal cancer, and head and neck tumors.

In some embodiments, the present application provides use of the dual-drug active ingredient nano-polymer prodrug in manufacture of a medicament for treating tumors.

In some other embodiments, the present application provides a combined medication method for treating a tumor in a patient in need thereof, including administering a therapeutically effective amount of the monomolecular nano-polymer prodrug or a formulation thereof to the patient.

The skilled person in the art can formulate the dual-drug active ingredient nano-polymer prodrug of the present application into any desired pharmaceutical formulation according to the methods known in the art. The dosage form of the pharmaceutical formulation of the present application can be any dosage form which is clinically suitable for treating the specified diseases, including solution, suspension, gel, lyophilized powder, capsule, tablet, etc. Preferably, the dosage form of the present application is suitable for injection, such as intravenous infusion.

When the nano-polymer micelles of the present application are formulated for administration by injection, such as intravenous infusion, the injectable formulation can be present in unit dosage forms, for example in ampoules, vials, prefilled syringes, or multi-dose containers.

It can be understood by those skilled in the art that, besides including the nano-polymer micelles, the pharmaceutical formulation of the present application can further include at least one pharmaceutically acceptable excipient as needed, such as an isotonic agent, a wetting agent, a solubilizing agent, an emulsifier, a preservative, a buffering agent, an acidifying agent, an alkalizing agent, an antioxidant, a chelating agent, a colorant, a complexing agent, a flavoring agent, a suspending agent, or a lubricant. These excipients are known in the art, and one or more appropriate excipients can be selectively incorporated into the pharmaceutical formulation of the present application by those skilled in the art.

The pharmaceutical formulation of the present application can be administered to a patient in need thereof through oral administration, intramuscular, intraperitoneal, intravenous, subcutaneous injection, etc. to treat the disease, such as the tumor, of the patient.

Clinicians in the field can choose and determine the dosage regimen of the nano-polymer micelles or the formulation thereof of the present application depending on the nature of the disease being treated, the time period of treatment, and the age and physical condition of the patient, so as to provide the desired therapeutic effect. The desired dose can be conveniently administered in a single dose or in multiple doses at appropriate intervals, and for example once, twice, or more appropriate doses per day can be administered.

The skilled person in the art can easily determine an applicable dosage of the nano-polymer micelles or the formulation thereof according to the type and amount of the drug active ingredient contained in the micelles.

In the treatment of the tumors, the nano-polymer micelles or the formulation thereof can be administered in combination with other chemotherapeutic agents and/or radiation.

### EXAMPLES

To further illustrate the present application, examples are provided below. These examples are merely for illustrating the present application and are not intended to limited to the scope of the present application.

Embodiments of the present application will be further described in detail below in conjunction with examples. It should be understood that the examples are merely for illustrating the present application and are not intended to limited to the scope of the present application. The experimental methods without indication of specific conditions should first refer to the guidelines given in the present application, and can also follow the experimental manuals or conventional conditions in the art, or follow the conditions suggested by the manufacturers, or refer to the experimental methods known in the art.

In the following specific examples, unless otherwise specified, measurements of raw material components may have slight deviations within the accuracy range of weighing. For temperature and time parameters, acceptable deviations due to instrument testing accuracy or operation accuracy are allowed.

Unless otherwise specified, the raw materials, experimental reagents and experimental instruments used in the following examples can be purchased from the market, the reaction conditions used are known in the art, and the identification or determination methods used are common methods in the art.

In the following examples, unless otherwise specified, Diboc is abbreviation for di-tert-butyl dicarbonate; DMAP is abbreviation for 4-dimethylaminopyridine; THF is abbreviation for tetrahydrofuran; DMF is abbreviation for N,N-dimethylformamide; EDC is abbreviation for 1-(3-dimethylaminopropyl)-3-ethylcarbodiimide hydrochloride; NHS is abbreviation for N-hydroxysuccinimide; BTC is abbreviation for bis(trichloromethyl)carbonate; Tween 80 is abbreviation for polysorbate 80; DACHPt is abbreviation for dichloro(1,2-diaminocyclohexane)platinum(II); CPT is abbreviation for camptothecin; and GSH is abbreviation for glutathione.

In the following examples, unless otherwise specified, “molecular weight” of poly(ethylene glycol) refers to the number average molecular weight.

In the following examples, unless otherwise specified, “molecular weight” of monomolecular nano-polymer refers to the number average molecular weight.

In the following examples, unless otherwise specified, “rt” in a reaction formula refers to room temperature.

In the following examples, unless otherwise specified, testing conditions of mass spectrometry are as follows: 1 mg/mL sample solution with dichloromethane or methanol as the solvent is prepared, 0.5 μL of the sample solution is dopped onto a sample stage and dried at room temperature, and the sample stage is introduced into an ion source (Bruker REFLEX III type, MALDI-TOF-MS) for testing.

In the following examples, unless otherwise specified, testing conditions of ¹H NMR are as follows: 10 mg/mL sample solution with deuterated chloroform (CDCl₃) as the solvent is prepared, and ¹H NMR spectra is established by using a superconducting pulse Fourier transform NMR spectrometer (Bruker AVANCE 500111 type) under a temperature of 25° C., with 64 scans, and tetramethylsilane (TMS) as an internal standard.

In the following examples, % (w/v) represents a percentage of mass to volume, and % (v/v) represents a percentage of volume to volume.

### Example 1. Preparation of a Drug Active Ingredient Prodrug Whose Molecule Includes a Free Hydroxyl Group, a Free Amino Group, or a Combination Thereof

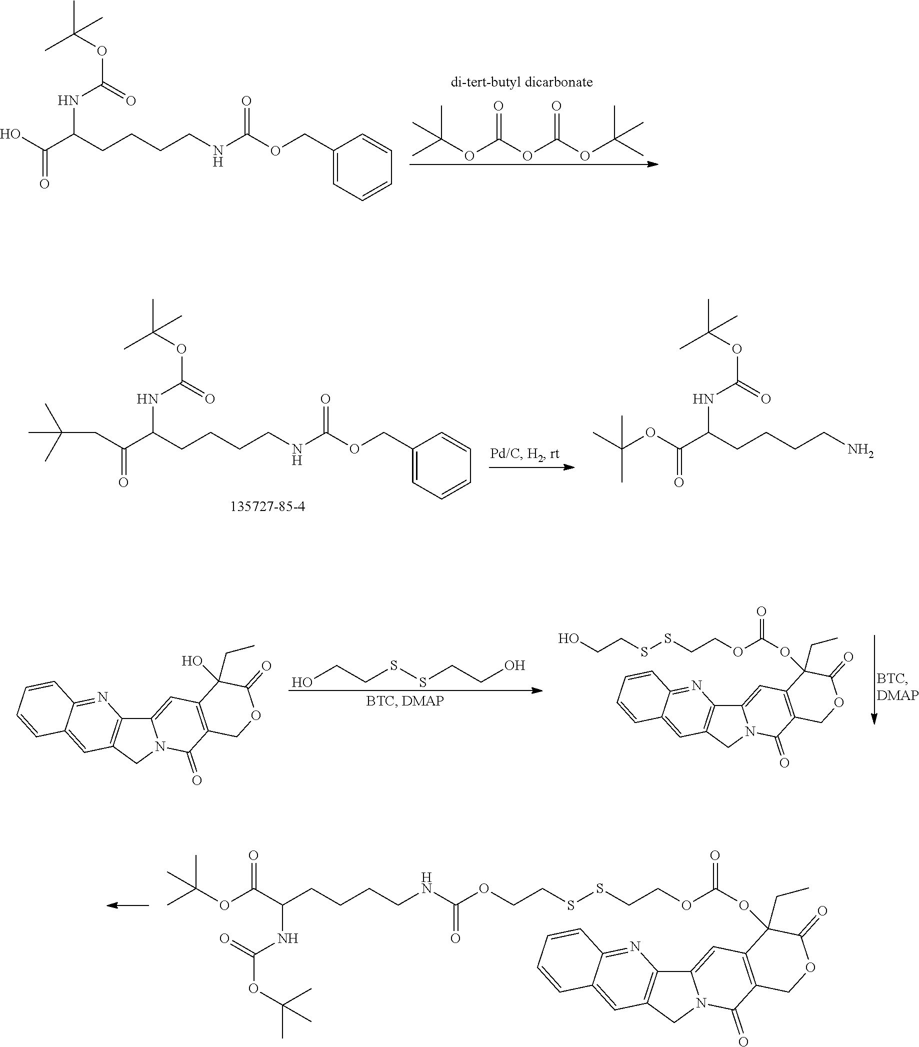

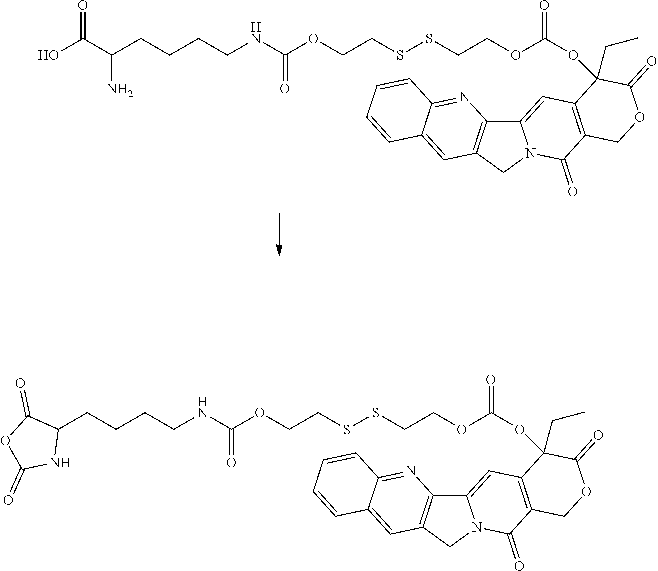 [SMILES: CCC1(OC(=O)OCCSSCCOC(=O)NCCCCC(N)C(=O)O)C(=O)OCc2c1cc1n(c2=O)Cc2cc3ccccc3nc2-1 ; CCC1(OC(=O)OCCSSCCOC(=O)NCCCCC2NC(=O)OC2=O)C(=O)OCc2c1cc1n(c2=O)Cc2cc3ccccc3nc2-1]

### 1.1 Syntheses of N-Boc-N′-Cbz-Lys-Otbu

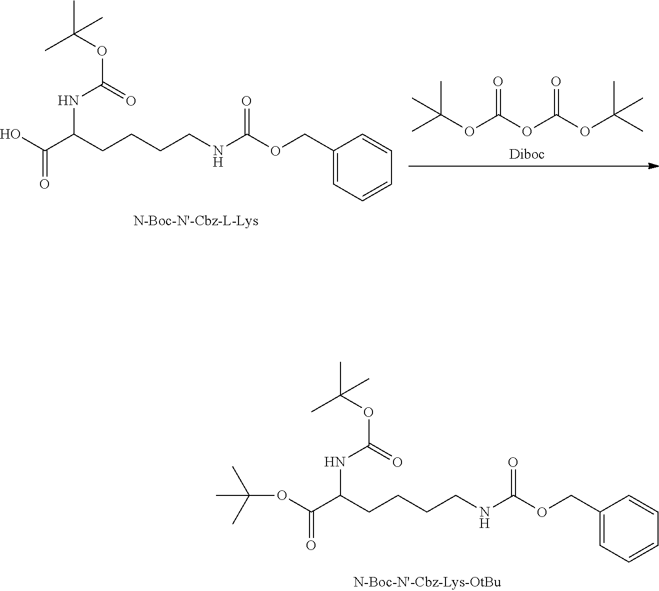

N-Boc-N′-Cbz-L-Lys (2 g, 5.26 mmol) was dissolved in 15 mL of chloroform, mixed with sodium bicarbonate solution (12 mL, 0.45 mmol/L), and stirred for 5 minutes under nitrogen gas protection. Then, a solution of di-tert-butyl dicarbonate (Diboc) in chloroform (1.22 g, 5.5 mmol, 9 mL) was added dropwise. The reaction solution was then refluxed for 90 minutes, and cooled to room temperature. The organic phase was separated, and the aqueous phase was extracted with chloroform. The organic phases were combined and evaporated under reduced pressure to remove the solvent, and then purified by column chromatography to obtain N-Boc-N′-Cbz-Lys-OtBu.

¹H NMR spectrum of the intermediate is shown in FIG. 4A.

### 1.2 Synthesis of Boc-Lys-OtBu

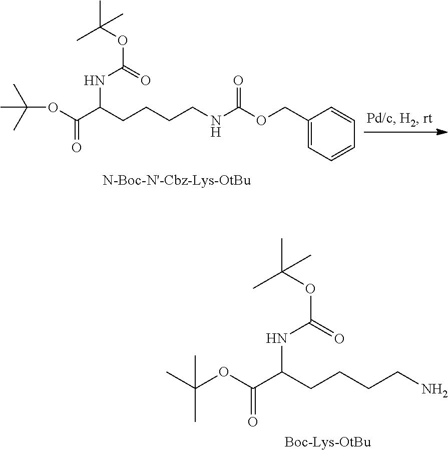 [SMILES: "Boc-Lys-OtBu" ; CC(C)(C)OC(=O)NC(CCCCNC(=O)OCc1ccccc1)C(=O)OC(C)(C)C ; CC(C)(C)OC(=O)NC(CCCCN)C(=O)OC(C)(C)C]

N-Boc-N′-Cbz-Lys-OtBu (436.5 mg, 1.0 mmol) was dissolved in methanol (5 mL), and added with 10% of palladium on carbon (Pd/C) (53.2 mg, 0.05 mmol). After mixing well, the reaction bottle was infused with hydrogen gas to remove the air inside, and the reactant was hydrogenated under balloon pressure, with stirring for reaction at room temperature for 2 hours. The solution was filtered through diatomite to remove insoluble substance. The filtrate was eluted with methanol, distilled under reduced pressure to remove the solvent, to obtain a yellow oil.

Mass spectrum of the intermediate is shown in FIG. 3A. ¹H NMR spectrum of the intermediate is shown in FIG. 4B.

### 1.3 Synthesis of CPT-ss-OH

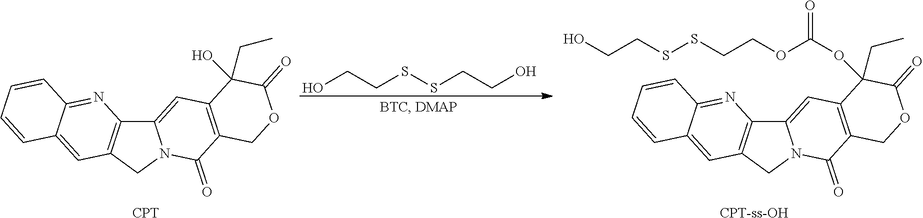

Camptothecin (500 mg, 1.43 mmol) was dispersed in anhydrous dichloromethane (80 mL). In an ice bath and under nitrogen gas protection, a solution of triphosgene (157 mg, 0.53 mmol) in anhydrous dichloromethane (3 mL) was added, and the mixture was stirred in the ice bath for 30 minutes to thoroughly dissolve the camptothecin. Then 4-dimethylaminopyridine (DMAP) (560 mg, 4.6 mmol) dissolved in anhydrous dichloromethane (10 mL) was added. Then the stirring was continued in the ice bath for 1 hour, and then in the dark at room temperature for another hour. 2-hydroxyethyl disulfide (1.75 mL, 14.3 mmol) dissolved in anhydrous THF (15 mL) was added under nitrogen gas protection. After mixing well, the reaction solution was stirred in the dark at room temperature for 24 hours. After completion of the reaction, additional 50 mL of dichloromethane was added. The mixed solution was washed three times each with 0.1 M HCl aqueous solution, saturated NaCl, and water sequentially. The organic phase was collected, dried with anhydrous Na₂SO₄, and distilled under reduced pressure by using a rotary evaporator to remove the organic solvent. The resulting yellow solid was purified by recrystallization in chloroform/methanol (3/10, v/v), to obtain light yellow crystals CPT-ss-OH.

Mass spectrum of the intermediate is shown in FIG. 3B. ¹H NMR spectrum of the intermediate is shown in FIG. 4C.

### 1.4 Synthesis of Boc-Lys-OtBu-ss-CPT

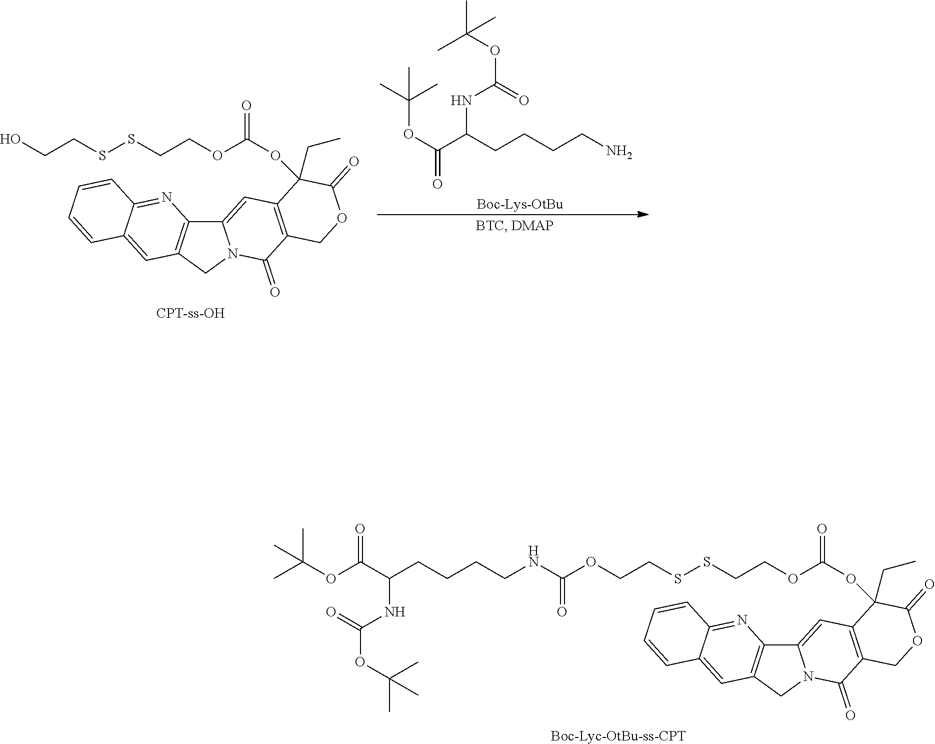 [SMILES: "Boc-Lys-OtBu" ; CCC1(OC(=O)OCCSSCCO)C(=O)OCc2c1cc1n(c2=O)Cc2cc3ccccc3nc2-1 ; CC(C)(C)OC(=O)NC(CCCCN)C(=O)OC(C)(C)C ; "CPT-ss-OH" ; CCC1(OC(=O)OCCSSCCOC(=O)NCCCCC(NC(=O)OC(C)(C)C)C(=O)OC(C)(C)C)C(=O)OCc2c1cc1n(c2=O)Cc2cc3ccccc3nc2-1 ; "Boc-Lyc-OtBu-ss-CPT"]

CPT-ss-OH (52.8 mg, 0.1 mmol) was dispersed in anhydrous dichloromethane (15 mL). In nitrogen environment, a solution of triphosgene (13.2 mg, 0.045 mmol) dissolved in anhydrous dichloromethane (1 mL) was added, and the mixture was stirred in an ice bath for 30 minutes to thoroughly dissolve the camptothecin. Then, 4-dimethylaminopyridine (DMAP) (39 mg, 0.32 mmol) dissolved in anhydrous dichloromethane (2 mL) was added. Then the stirring was continued in the ice bath for 1 hour, and then in the dark at room temperature for another hour. Boc-Lys-OtBu (45.3 mg, 0.15 mmol) dissolved in anhydrous dichloromethane (1 mL) was added under nitrogen gas protection. After mixing well, the reaction solution was stirred in the dark at room temperature for 24 hours. After completion of the reaction, additional 50 mL of dichloromethane was added. The mixed solution was washed three times each with 0.1 M HCl aqueous solution, saturated NaCl and water sequentially. The organic phase was collected, dried with anhydrous Na₂SO₄, and separated by column chromatography to obtain a light yellow crystal Boc-Lys-OtBu-ss-CPT.

¹H NMR spectrum of the intermediate is shown in FIG. 4D.

### 1.5 Synthesis of Lys-ss-CPT

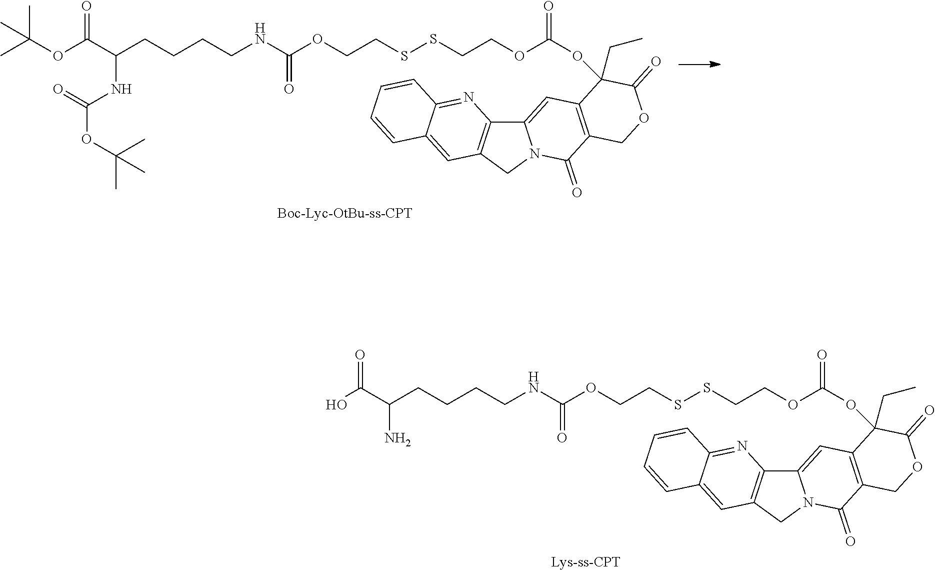 [SMILES: CCC1(OC(=O)OCCSSCCOC(=O)NCCCCC(NC(=O)OC(C)(C)C)C(=O)OC(C)(C)C)C(=O)OCc2c1cc1n(c2=O)Cc2cc3ccccc3nc2-1 ; "Boc-Lyc-OtBu-ss-CPT" ; CCC1(OC(=O)OCCSSCCOC(=O)NCCCCC(N)C(=O)O)C(=O)OCc2c1cc1n(c2=O)Cc2cc3ccccc3nc2-1 ; "Lys-ss-CPT"]

Boc-Lys-OtBu-ss-CPT powder (85.7 mg, 0.1 mmol) was dissolved in dichloromethane (2 mL), mixed with trifluoroacetic acid (2 mL), and reacted at room temperature for 2 hours. The solution was distilled under reduced pressure to remove the solvent, then dissolved with dichloromethane, and washed with saturated sodium bicarbonate. The organic phase was collected and dried to obtain Lys-ss-CPT.

### 1.6 Synthesis of NCA-Lys-ss-CPT

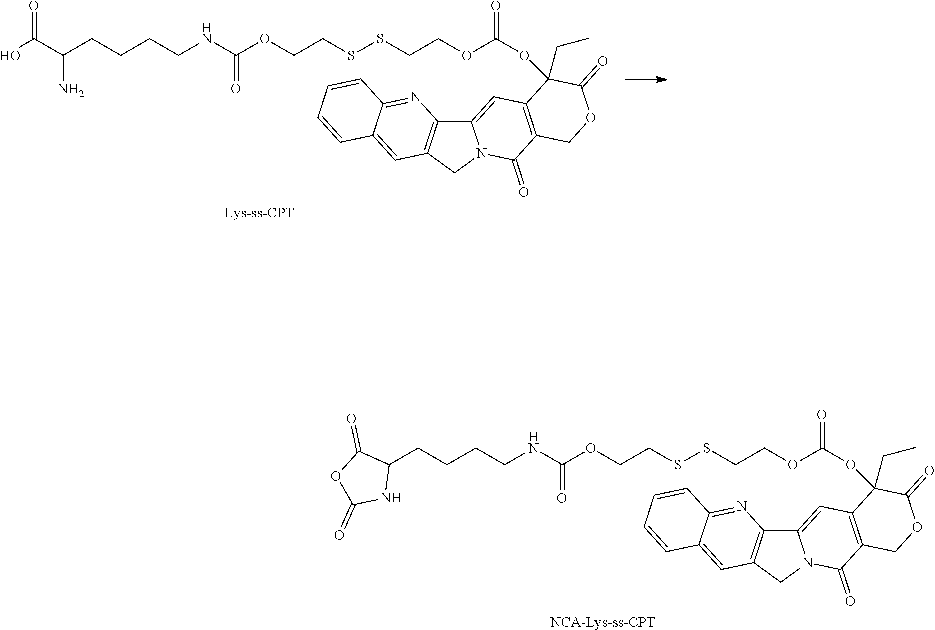 [SMILES: CCC1(OC(=O)OCCSSCCOC(=O)NCCCCC(N)C(=O)O)C(=O)OCc2c1cc1n(c2=O)Cc2cc3ccccc3nc2-1 ; "Lys-ss-CPT" ; CCC1(OC(=O)OCCSSCCOC(=O)NCCCCC2NC(=O)OC2=O)C(=O)OCc2c1cc1n(c2=O)Cc2cc3ccccc3nc2-1 ; "NCA-Lys-ss-CPT"]

Lys-ss-CPT (0.1 mmol) was dissolved in dichloromethane (2 mL), mixed with a solution of triphosgene (0.2 mmol) in dichloromethane (2 mL), and reacted at room temperature for 2 hours. The solution was distilled under reduced pressure to remove the solvent, then dissolved in tetrahydrofuran at a temperature of 60° C., and then added with an appropriate amount of n-hexane. The solution was placed in a refrigerator at 4° C. for crystallization, and white needle-like crystals (i.e., NCA-Lys-ss-CPT) were collected.

Mass spectrum of the camptothecin prodrug is shown in FIG. 3C.

### Example 2. Preparation of a Platinum-Based Drug Active Ingredient Prodrug

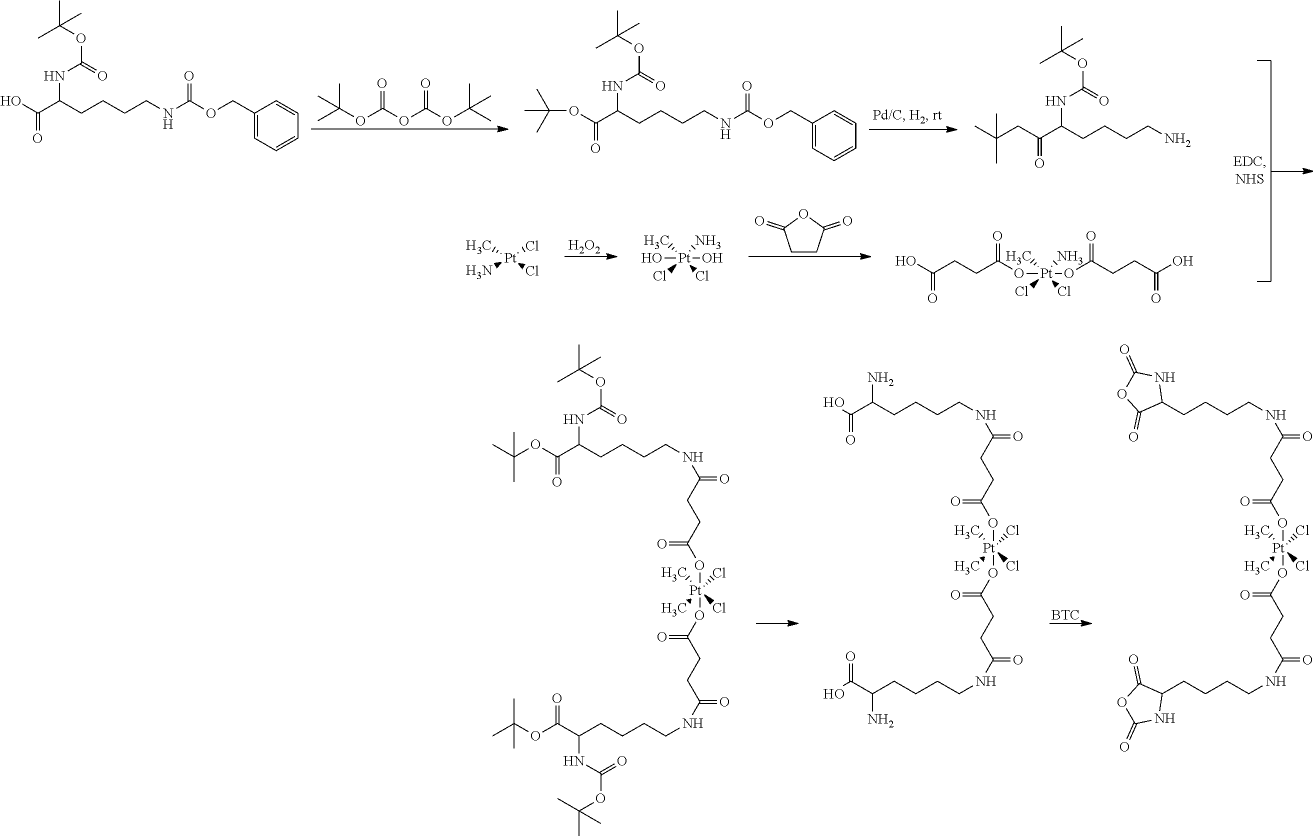

### 2.1 Synthesis of Compound (4)

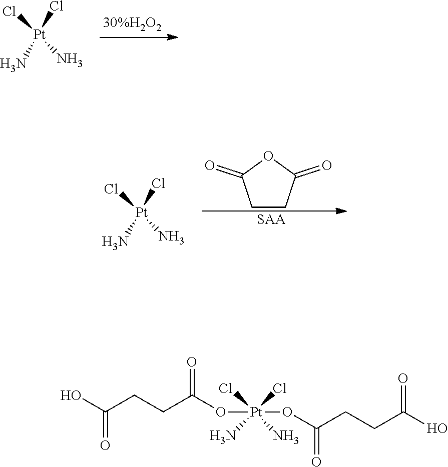

Cisplatin (1.0 g, 3.33 mmol) was dispersed in distilled water, and 30% hydrogen peroxide (H₂O₂) (20 ml) was added. The mixture was stirred in the dark at 70° C. for 5 hours until becoming clear, cooled to room temperature, and then placed in a refrigerator at 4° C. for recrystallization. The formed crystals were filtered, and the filter cake was washed with ice water, ethanol, and diethyl ether sequentially, then dried to obtain yellow crystalline compound (1).

Compound (1) (0.5 mg, 1.5 mmol) and succinic anhydride (0.62 g, 6.15 mmol) were dissolved in DMF (20 ml), and the mixture was stirred overnight in the dark at 70° C., followed by distillation under reduced pressure to remove the solvent, washed with methanol and acetone, and then dried to obtain light yellow compound (4).

¹H NMR spectrum of compound (4) is shown in FIG. 4E.

### 2.2 Synthesis of N-Boc-N′-Cbz-Lys-OtBu and Boc-Lys-OtBu

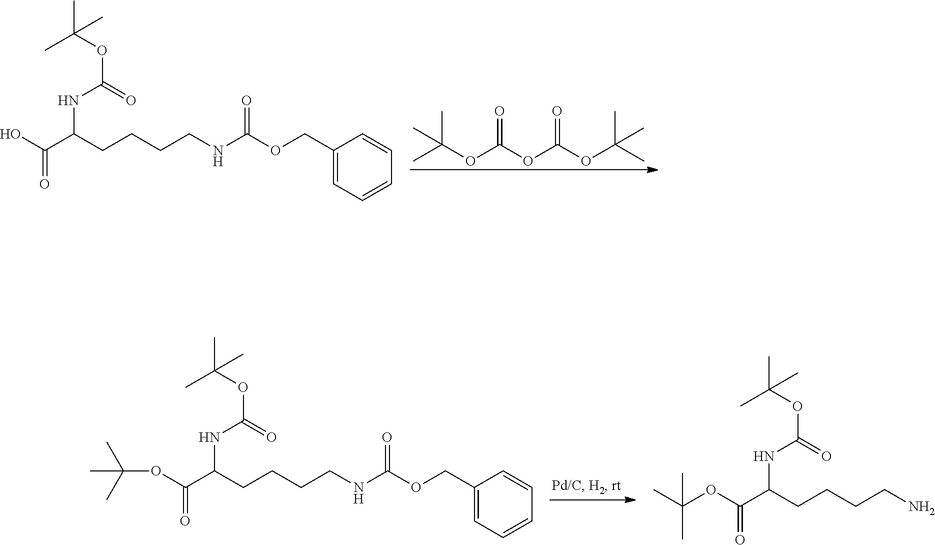

### 2.3 Synthesis of N-Boc-N′-Cbz-Lys-OtBu

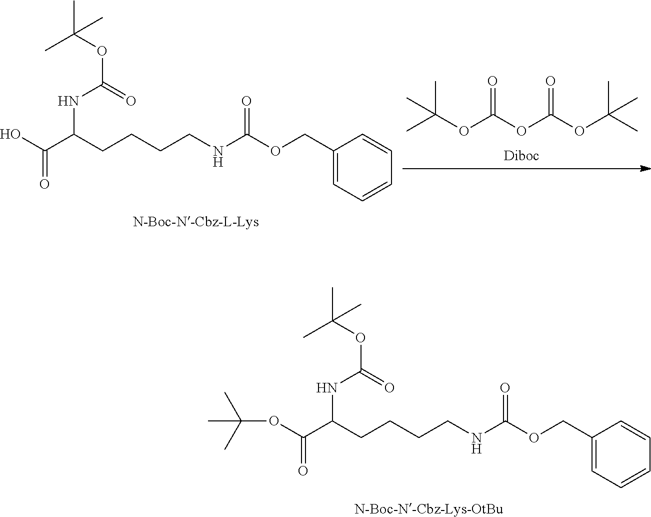

N-Boc-N′-Cbz-L-Lys (2 g, 5.26 mmol) was dissolved in chloroform (15 mL) and mixed with sodium bicarbonate solution (12 mL, 0.45 mmol/L). The mixed solution was stirred for 5 minutes under nitrogen gas protection. A Diboc-containing chloroform solution (1.22 g, 5.5 mmol, 9 mL) was added dropwise. The solution was then refluxed for 90 minutes, and cooled to room temperature. The organic phase was separated, and the aqueous phase was extracted with chloroform. The organic phases were combined and evaporated under reduced pressure to remove solvent, and then purified by column to obtain N-Boc-N′-Cbz-Lys-OtBu.

### 2.4 Synthesis of Boc-Lys-OtBu

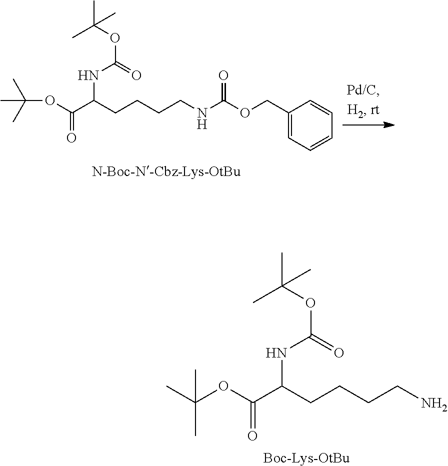 [SMILES: "Boc-Lys-OtBu" ; CC(C)(C)OC(=O)NC(CCCCNC(=O)OCc1ccccc1)C(=O)OC(C)(C)C ; CC(C)(C)OC(=O)NC(CCCCN)C(=O)OC(C)(C)C]

N-Boc-N′-Cbz-Lys-OtBu (436.5 mg, 1.0 mmol) was dissolved in methanol (5 mL), and added with 10% of palladium on carbon (Pd/C) (53.2 mg, 0.05 mmol). After mixing well, the reaction bottle was infused with hydrogen gas to remove the air inside, and the reactant was hydrogenated under balloon pressure, with stirring for reaction at room temperature for 2 hours. The solution was filtered through diatomite to remove insoluble substance. The filtrate was eluted with methanol, distilled under reduced pressure to remove solvent to obtain a yellow oil.

### 2.5 Synthesis of Compound (5)

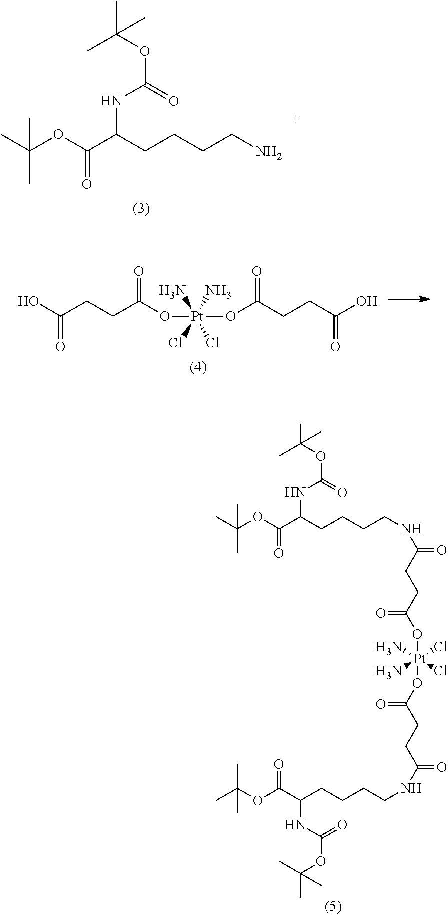

Compound (4) (0.12 mmol) was dissolved in anhydrous DMF (5 mL), mixed with a solution of 1-(3-dimethylaminopropyl)-3-ethylcarbodiimide hydrochloride (EDC) (0.15 mmol) and DMAP (0.05 mmol) in anhydrous DMF (5 mL), and then reacted for 30 minutes. After that, a solution of compound (3) (0.1 mmol) in anhydrous DMF (5 mL) was added and mixed. The mixed solution was stirred at room temperature for 6 hours, and then separated by column chromatography to obtain a light yellow solid (5).

### 2.6 Synthesis of Compound (6)

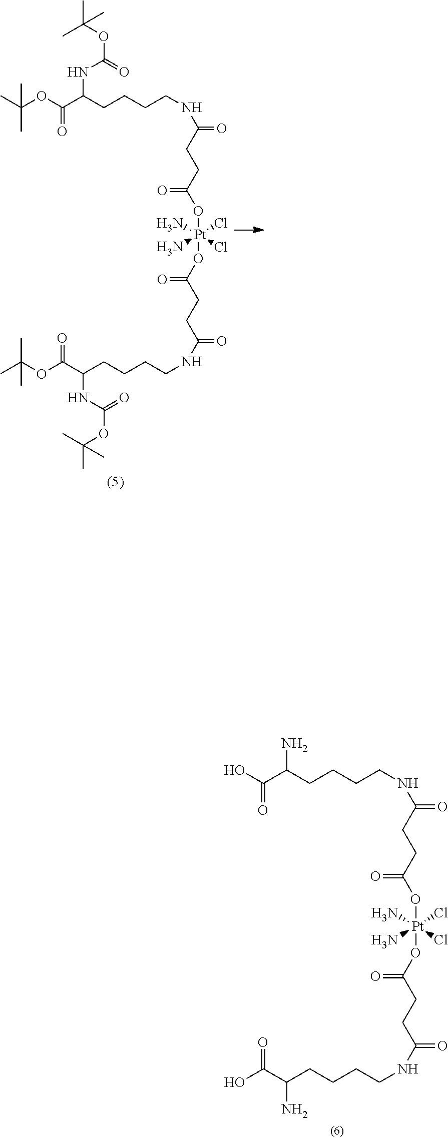

Compound (5) (0.1 mmol) was dissolved in DMF (2 mL), mixed with trifluoroacetic acid (2 mL), and reacted at room temperature for 2 hours. The reacted solution was distilled under reduced pressure to remove the solvent, then washed with methanol and acetone sequentially, and dried to obtain light yellow compound (6).

### 2.7 Synthesis of Compound (7)

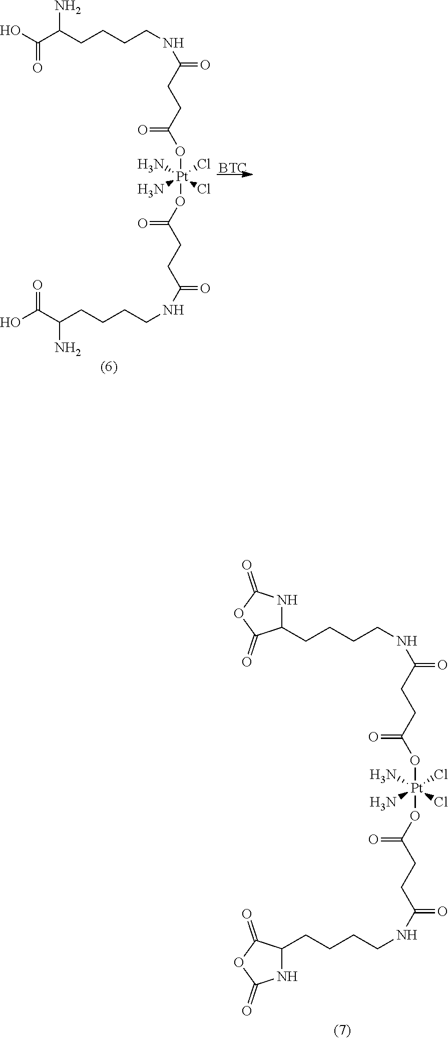

Compound (6) (0.1 mmol) was dissolved in DMF (2 mL), mixed with a solution of triphosgene (0.2 mmol) in DMF (2 mL), and then reacted at room temperature for 2 hours. The reacted solution was distilled under reduced pressure to remove the solvent, and dissolved in tetrahydrofuran at 60° C., and then an appropriate amount of n-hexane was added. The solution was placed in a refrigerator at 4° C. for crystallization, and white needle-like crystals (7) NCA-Pt-NCA were collected. Mass spectrum of compound (7) is shown in FIG. 3D.

### Example 3. Preparation of Drug-Loaded Monomolecular Nano-Polymer and Micelles Thereof

In this example, the drug-loaded monomolecular nano-polymer is a dual-drug monomolecular nano-polymer and can be used as a nano-polymer prodrug. The drug-loaded monomolecular nano-polymer includes a tetravalent structural unit represented by formula (I-2), a monovalent structural unit represented by formula (III-2), and a bivalent structural unit represented b formula (II-2):

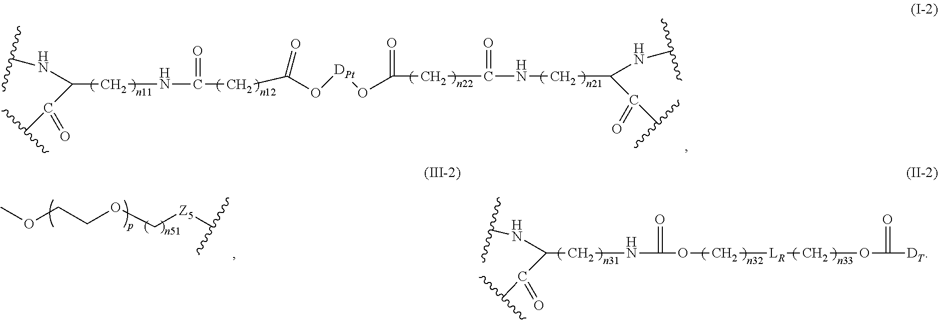

Further, in this example, n11 and n21 at each occurrence are each 4, and n12 and n22 at each occurrence are each 2.

n31 at each occurrence is 4, n32 at each occurrence is 2, and n33 at each occurrence is 2. L_{R} at each occurrence is-S—S—.

n51 at each occurrence is 3, and Z₅ at each occurrence is NH.

Furthermore, D_{Pt} is the residue of cisplatin, and D_{T} is the residue of camptothecin.

Furthermore, p is approximately equal to 113, and the number average molecular weight of corresponding mPEG is about 5000 Da.

The drug-loaded monomolecular nano-polymer was prepared by using the monomer represented by formula (I-3), the monomer represented by formula (II-3), and the monomer represented by formula (II-3).

Further, NCA-Pt-NCA prepared in Example 2 was used as the monomer represented by formula (I-3), NCA-Lys-ss-CPT prepared in Example 1 was used as the monomer represented by formula (II-3), and the monomer represented by formula (II-3) had

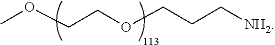

The reaction scheme for preparing the drug-loaded monomolecular nano-polymer is shown as below, in which P101 is labeled for the drug-loaded monomolecular nano-polymer.

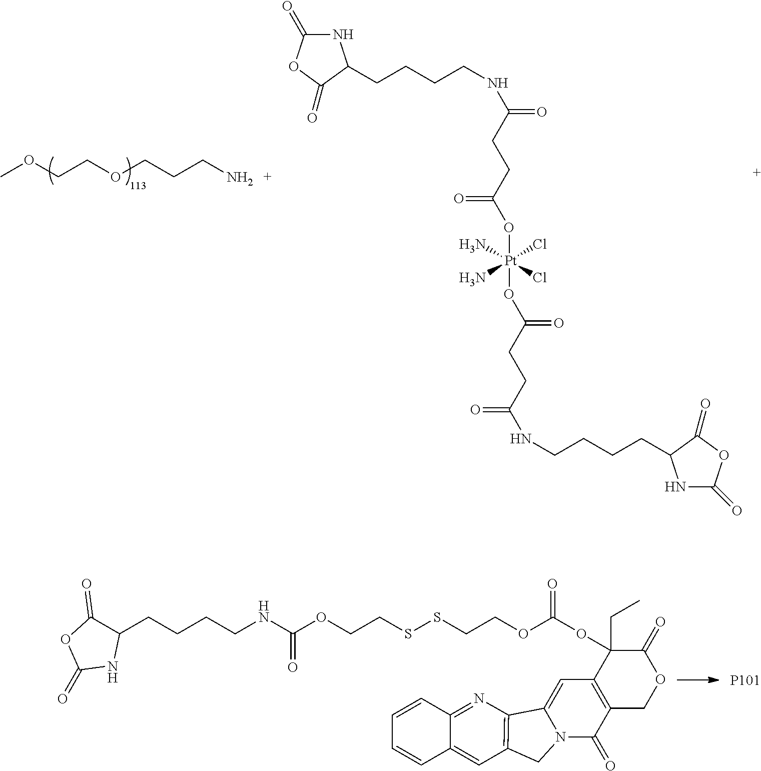

In the above scheme, m can be in a range from 0 to 100, n can be in a range from 1 to 100, k can be in a range from 1 to 1000, but theses ranges are not limiting.

Methoxy-poly(ethylene glycol)-amino (MeO-PEG-NH₂) (0.1 g, 0.01 mmol) was added in benzene (3 mL), stirred to completely dissolve the PEG, then frozen in liquid nitrogen, and dried under vacuum in a cold trap for 6 hours. Then, in a glove box, the dried polyethylene glycol was dissolved in anhydrous DMF (2 mL) and stirred uniformly. NCA-Lys-ss-CPT (0.50 mmol) and NCA-Pt-NCA (0.18 mmol) were dissolved in anhydrous DMF (2 mL), and the resulting solution was slowly added dropwise to the reaction system. The reaction tube was then sealed, taken out from the glove box, and continuously stirred in an oil bath at a temperature of 35° C. for 72 hours. The reaction product was slowly added dropwise into anhydrous diethyl ether to obtain a white precipitate, the supernatant was discarded, and this process was repeated three times to obtain a purified product. The purified product was then vacuum dried in a vacuum pot for 6 hours. The dried solid was dissolved in dimethyl sulfoxide (DMSO) (2 mL), introduced into a dialysis bag (MWCO: 100 kDa), dialyzed in ultrapure water for two days (during which water was changed five times), freeze-dried, to obtain a final product P101.

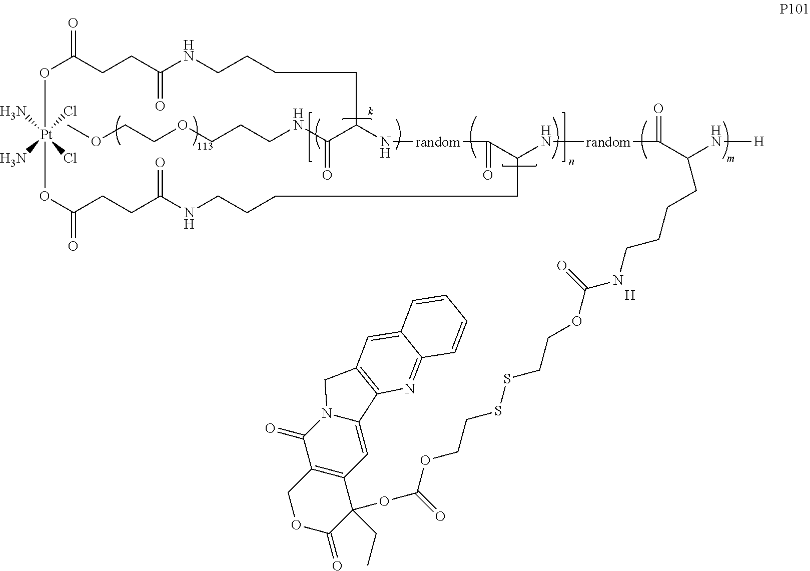

In the above formula, m can be in a range from 0 to 100, n can be in a range from 1 to 100, k can be in a range from 1 to 1000, but theses ranges are not limiting.

In addition, by using the above method for preparing the dual-drug (camptothecin and cisplatin) polymer prodrug nano-micelles, and replacing camptothecin with paclitaxel or resiquimod, dual-drug (paclitaxel and cisplatin) polymer prodrug nano-micelles and dual-drug (resiquimod and cisplatin) polymer prodrug nano-micelles were prepared.

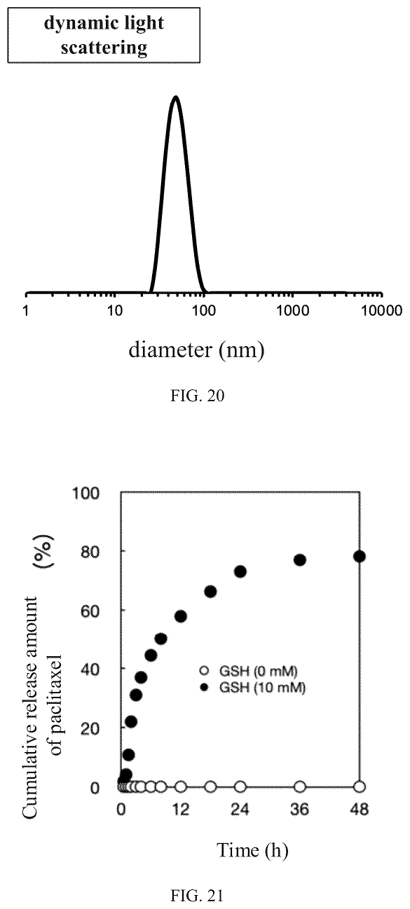

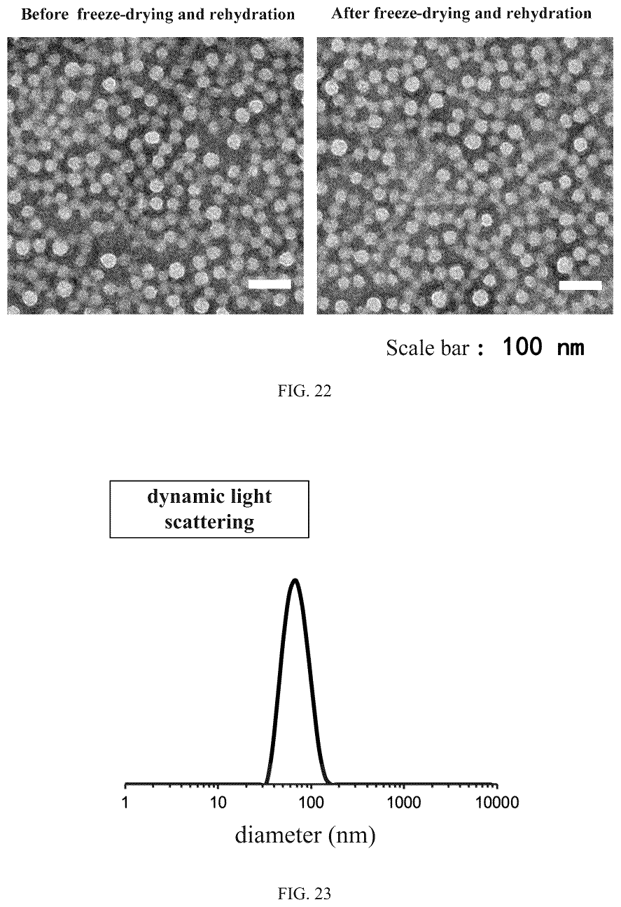

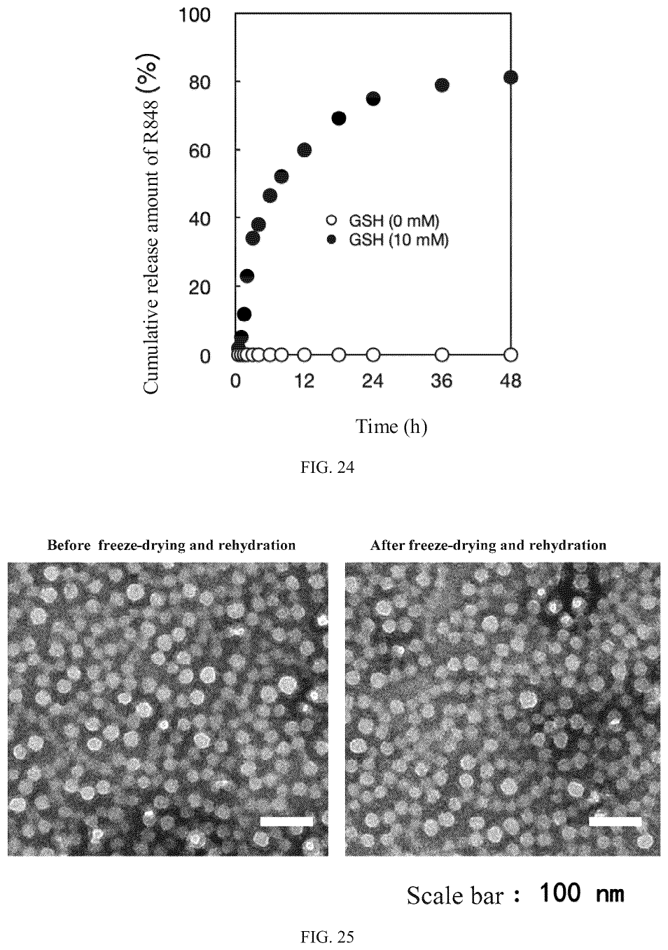

The dynamic light scattering characteristics and drug release characteristics of the dual-drug (paclitaxel and cisplatin) polymer prodrug nano-micelles and the dual-drug (resiquimod and cisplatin) polymer prodrug nano-micelles are shown in FIGS. 20 to 25B.

### Example 4. Characterization of Dual-Drug Monomolecular Polymer Prodrug Nano-Micelles

### 4.1. Control Micelles Used in the Present Application

### A: CPT@PEG-PLA

Refer to the master's thesis, “Jin Tao, Preparation of hydroxycamptothecin MePEG-PLA nanoparticles and in vitro anti-tumor research, Zhejiang Chinese Medical University, May 1, 2013”.

B: Cisplatin @ PEG-Gluc (ss-CPT)

Refer to patent document CN109908084A, entitled in “Platinum cross-linked camptothecin prodrug micelle nano drug and preparation method and application thereof”.

### C: Cisplatin @PEG-PGlu

Refer to patent documents CN101203549B, CN100457185C, and CN100344293C.

D: Cisplatin, which is Commercially Available.

4.2 Physical and chemical characterization: Molecular weight, GPC (SEC), etc.

The freeze-dried product was dissolved in water (1 mg/mL) and characterized for molecular weight distribution using GPC (superdex200). As shown in FIGS. 5A and 5B, the synthesis of the high molecular weight product was successful.

The freeze-dried product was dissolved in water (1 mg/mL). The molecular weight of the product was quantified by using an analytical ultracentrifuge, which was about 1030 kDa. That is, the post-polymerization molecular weight was measured by analytical ultracentrifugation.

Results: the molecular weight of PEG (p in formula (III-2) was about 113) is 5 kDa, and the molecular weight of the nano particles is 1030 kDa.

The freeze-dried product was dissolved in water (1 mg/mL). The diffusion time of the product was quantified by using a fluorescence correlation spectrometer (FCS), which was about 7600 μs. That is, the post-polymerization molecular weight was measured by fluorescence correlation spectroscopy.

Results: The diffusion time for PEG is 121 μs, and for the formed nano particles is 7600 μs.

### 4.3 Particle Size Characterization

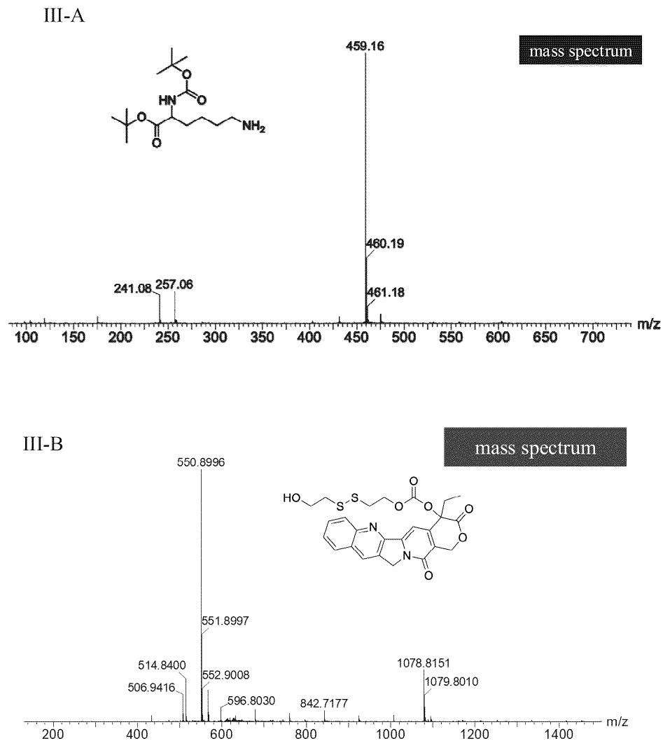

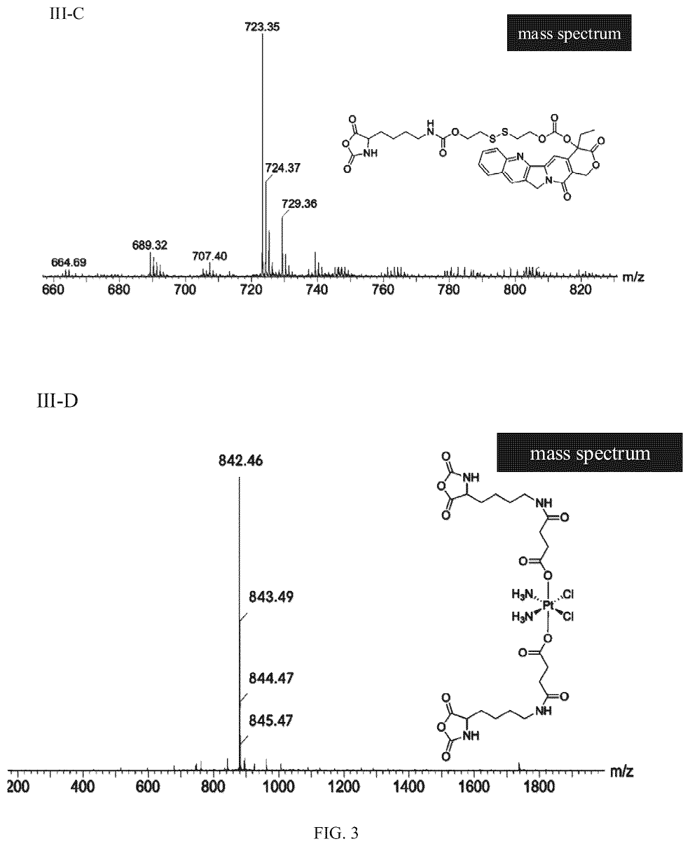

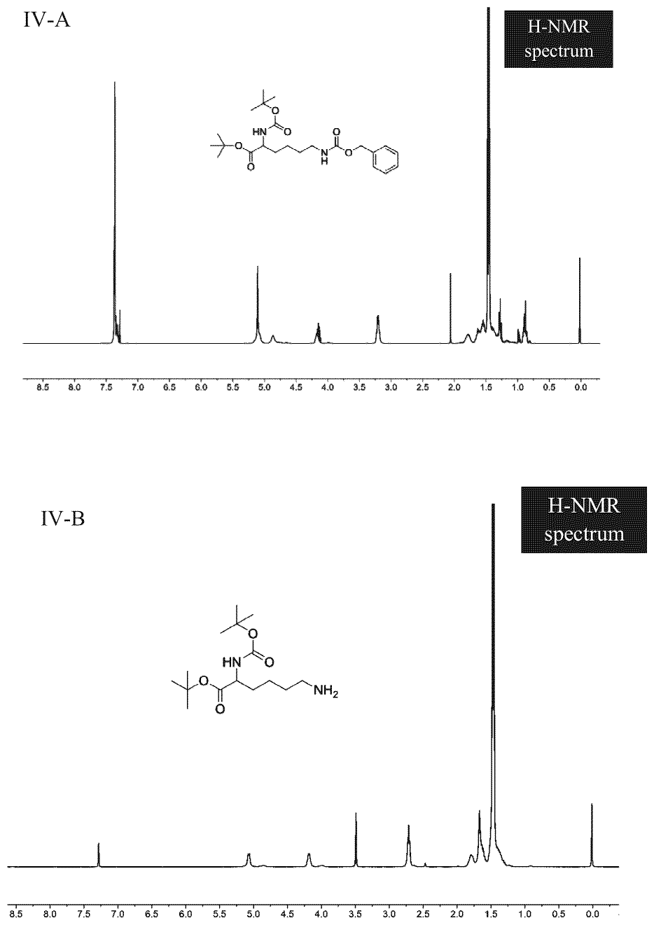

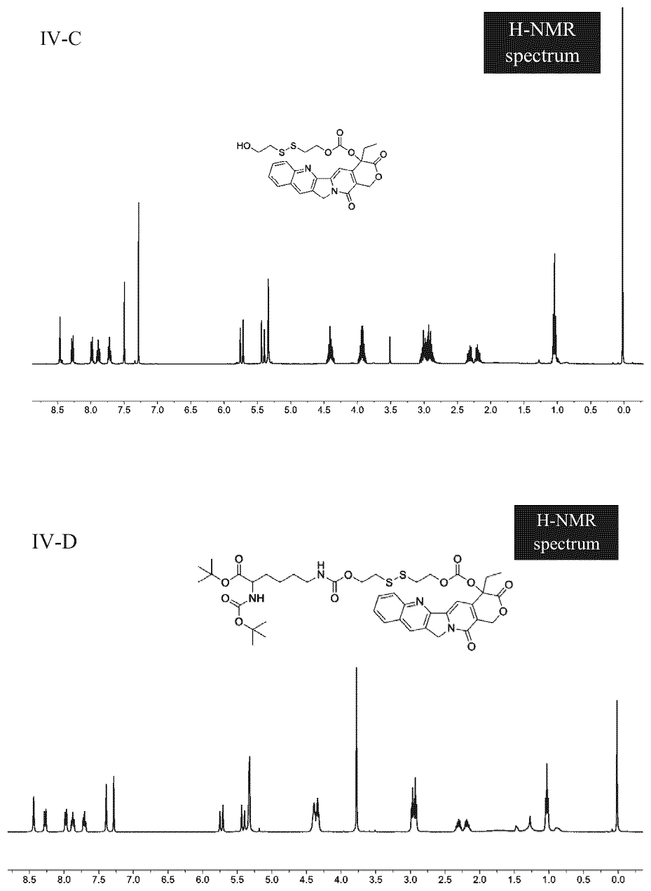

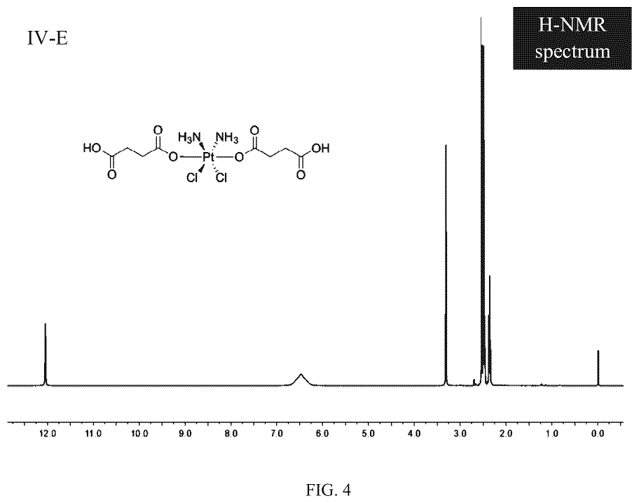

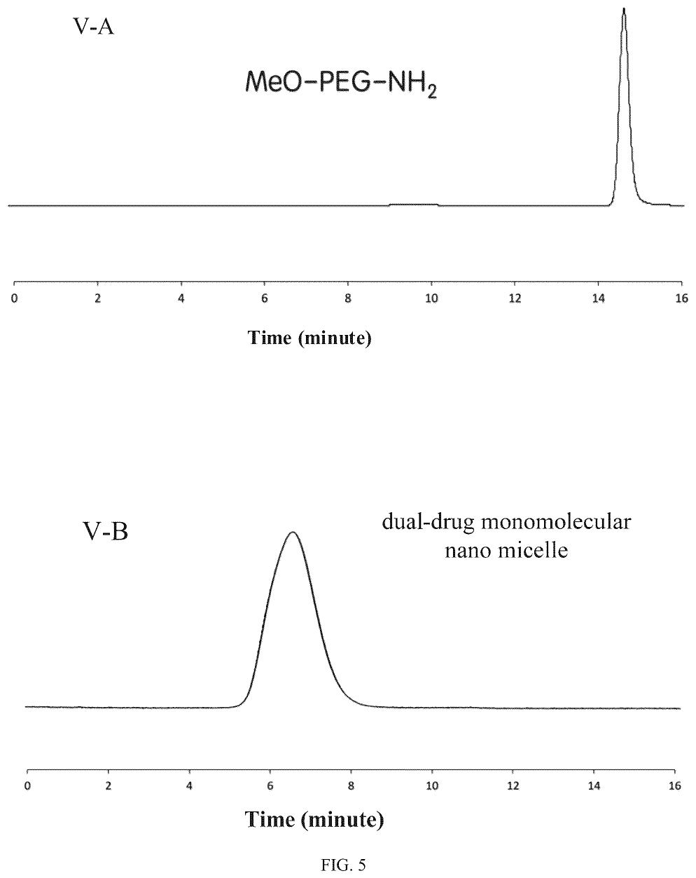

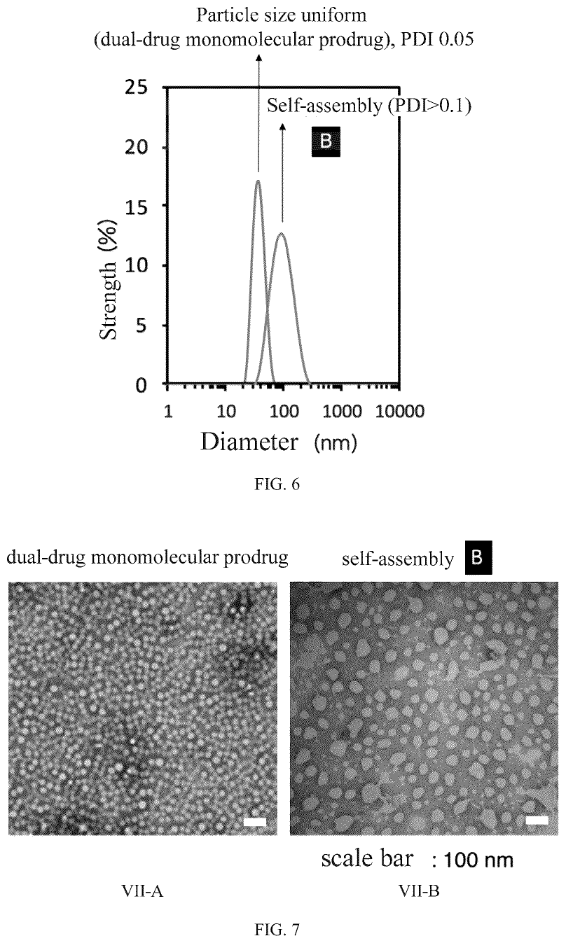

A nano-formulation (0.01 mg/mL) in a phosphate buffer saline (PBS) solution (10 mM, pH 7.4) was prepared. The particle size and polydispersity index (PDI) of the nano-formulation were characterized by using a dynamic light scattering instrument. The results are shown in FIG. 6. It can be seen from FIG. 6 that the dual-drug monomolecular nano-polymer micelles of the present application have particle sizes ranging from 21.5 nm to 52.7 nm, an average particle size of 33.6 nm, and a polydispersity index (PDI) of about 0.05; while the control micelles have particle sizes ranging from 32.3 nm to 264.1 nm, an average particle size of about 96.7 nm, and a polydispersity index (PDI) greater than 0.1, specifically in a range from 0.18 to 0.30.

### 4.4. TEM Morphology Test

A nano-formulation aqueous solution (0.01 mg/mL) was prepared. A TEM copper grid was completely immersed in the solution, kept for 30 minutes, and taken out using tweezers. Then, excess solution on the copper grid was removed with a filter paper. The copper grid was air-dried naturally, then negatively stained with 2 wt % phosphotungstic acid aqueous solution for 2 minutes, air-dried naturally again, and then observed and photographed under in a transmission electron microscope. The results are shown in FIGS. 7A and 7B.

It can be seen that the nano particles obtained by chemical polymerization exhibit a high degree of size uniformity, while the control nano particles obtained by self-assembly exhibit a low degree of size uniformity.

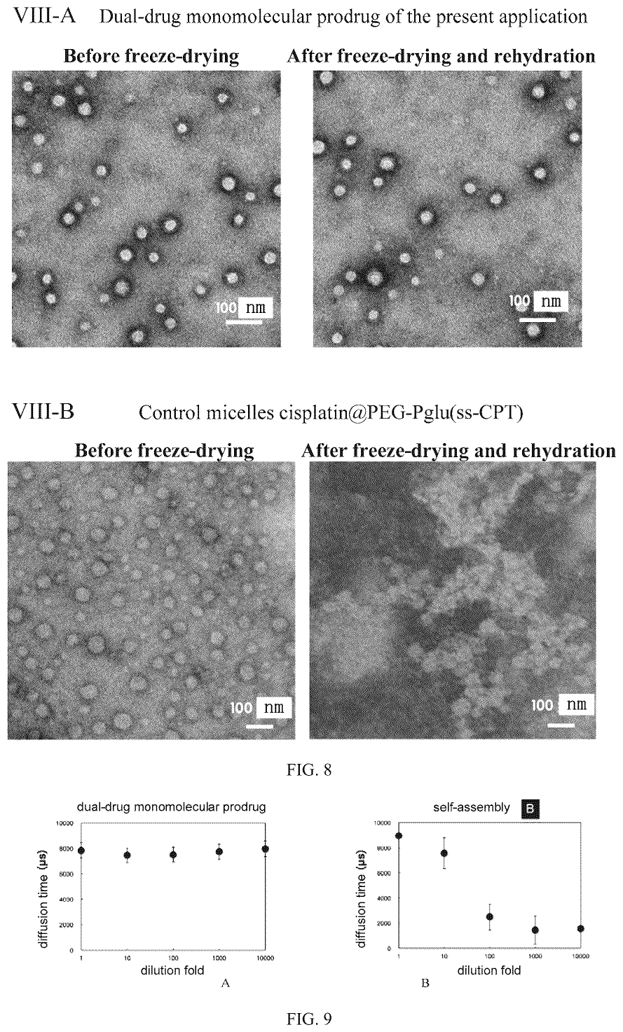

The solution subjected to dialysis undergone three times of freeze-drying and rehydration (1 mg/mL). Comparing the morphologies before and after the dialysis and three times of freeze-drying and rehydration, the dual-drug monomolecular polymer prodrug nano-micelles can maintain their original morphology, while the control nano-formulation formed by self-assembly cannot withstand the freeze-drying and rehydration process. The results are shown in FIGS. 8A and 8B.

Conclusion: The dual-drug monomolecular polymer prodrug nano-micelles obtained by chemical polymerization has an excellent colloidal solution stability, can tolerate the freeze-drying and rehydration process, and maintain structural stability; while the control nano particles obtained by self-assembly cannot tolerate the freeze-drying and rehydration process, and cannot retain their original structure after rehydration.

### 4.5 Fluorescence Correlation Spectroscopy (FCS) Test for Colloid Dynamics Characteristics

A solution of dual-drug monomolecular polymer prodrug nano-micelles (0.1 mg/mL) and a solution of self-assembled nano micelles (0.1 mg/mL) were respectively diluted with water, and their colloid dynamics characteristics were tested by using fluorescence correlation spectroscopy (FCS). The results are shown in FIGS. 9A and 9B.

It can be seen from the results that the dual-drug monomolecular polymer prodrug nano-micelles obtained by chemical polymerization can tolerate the dilution process, and can maintain their original structure even in a 10,000-fold dilution; while the nano-formulation obtained by self-assembly cannot tolerate the dilution process, with their self-assembled structure dissociated after a 100-fold dilution.

### 4.6 Small-Angle X-Ray Scattering (SAXS) Test

A solution of dual-drug monomolecular polymer prodrug nano-micelles (0.1 mg/mL) was subjected to small-angle X-ray scattering (SAXS) testing. The result is shown in FIG. 10.

It can be seen from the result that the synthesized product has a nano-sized micelle structure. The core radius of the micelle is about 6.4 nm, and the thickness of the PEG shell of the micelle is about 9.8 nm. The diameter of the micelles is about 32.4 nm.

### 4.7 Colloidal Stability

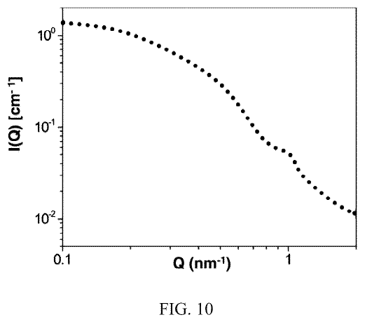

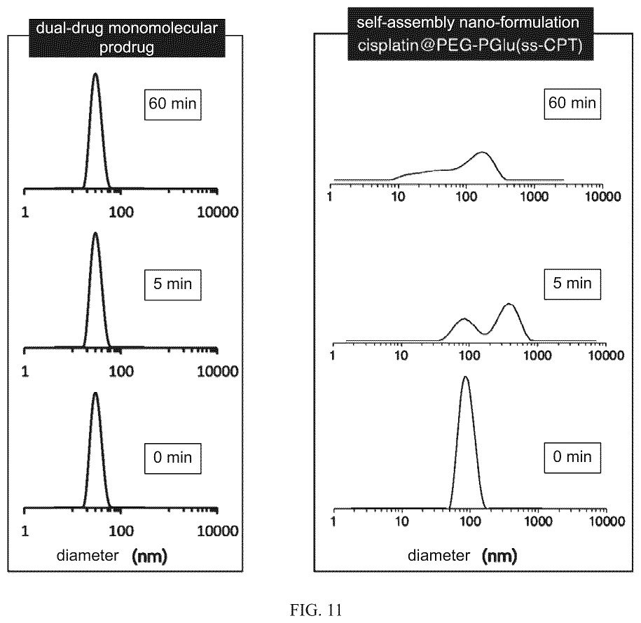

A pharmaceutical formulation dispersed in an aqueous solution and circulating in the bloodstream will be subjected to mechanical shear forces. Therefore, the colloidal stability of the nano-formulation is of great significance to its pharmacokinetics and its industrial processing. The present test investigated the colloidal structure stability of the dual-drug monomolecular polymer prodrug nano-micelles of the present application and the self-assembled nano-formulation cisplatin@PEG-PGlu(ss-CPT) under ultrasonic treatment, using dynamic light scattering to test their colloidal stability. The ultrasonic parameters (1.0 MHz, 9.9 W) and ultrasonic time period (0 min, 5 mins, and 60 mins) were set. The solutions after the ultrasonic treatment were analyzed by using a dynamic light scattering instrument to determine the particle size distribution. The results are shown in FIGS. 11A and 11B.

Conclusion: The dual-drug monomolecular nano-polymer prodrug micelles obtained by chemical polymerization can tolerate ultrasonic treatment very well, maintaining stable particle size; while the self-assembled nano-formulation cisplatin@PEG-PGlu(ss-CPT) cannot tolerate ultrasonic treatment, showing obvious structural deformation.

It can be seen from FIGS. 11A and 11B that for the dual-drug monomolecular nano-polymer micelles of the present application, before the ultrasonic treatment, the average particle size is 33.6 nm, the particle sizes range from 21.5 nm to 52.7 nm, and the polydispersity index (PDI) is about 0.05; after 60 minutes of ultrasonic treatment, the average particle size is 33.9 nm, the particle sizes range from 22.1 nm to 53.4 nm, and the polydispersity index (PDI) is about 0.05. Thus, these results indicate that the dual-drug monomolecular nano-polymer micelles can tolerate the ultrasonic treatment very well, maintaining stable particle size.

For the self-assembled nano-formulation cisplatin@PEG-PGlu(ss-CPT) control micelles, before the ultrasonic treatment, the average particle size is 96.7 nm, the particle sizes range from 32.3 nm to 264.1 nm, and the polydispersity index (PDI) is about 0.18; after 60 minutes of ultrasonic treatment, the average particle size is 342 nm, the particle sizes range from 7.6 nm to 464.1 nm, and the polydispersity index (PDI) is about 0.64. Thus, these results indicate that the self-assembled nano-formulation cisplatin@PEG-PGlu(ss-CPT) control micelles cannot tolerate the ultrasonic treatment, showing obvious structural deformation.

### Example 5. Dual-Drug Monomolecular Polymer Prodrug Nano-Micelles in Response to Intracellular Reducing Microenvironment—Investigation of Active Drug Release

### 5.1. Drug Release Mechanism

The active drug CPT is released according to the following molecular mechanism.

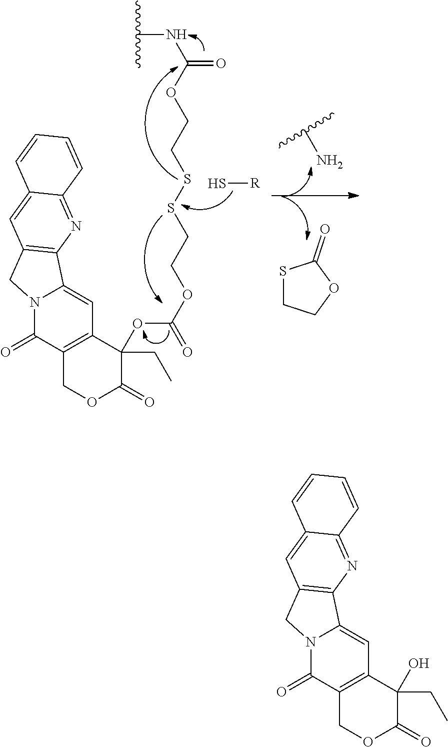

The monomolecular nano-polymer of the present application releases an active Pt(II) drug due to reduction of the tetravalent platinum in the cell according to the following molecular mechanism.

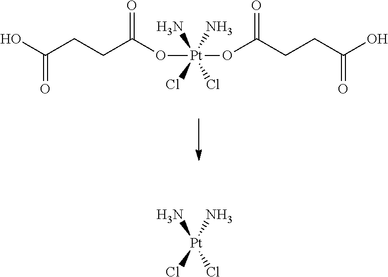

### 5.2. In Vitro Simulated Drug Release Behavior

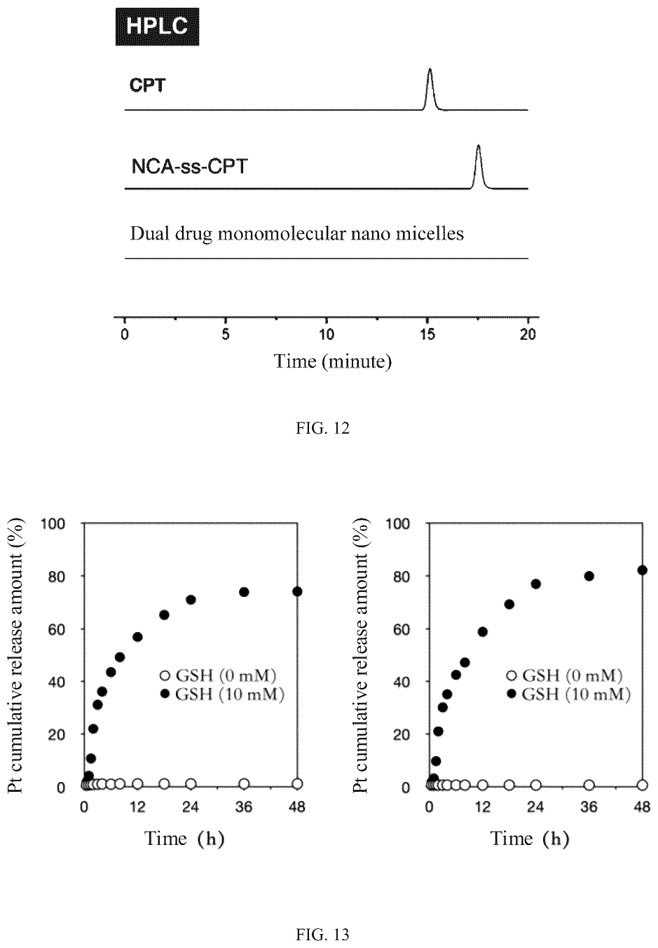

The drug release behavior of the dual-drug monomolecular polymer prodrug nano-micelles was investigated under different conditions: (1) extracellular microenvironment: pH 7.4, GSH (0 mM); and (2) intracellular microenvironment: pH 7.4, GSH (10 mM). The PBS solution containing the above nano-formulation (10 mM) was introduced into dialysis bags (MWCO: 10,000). The dialysis bags were respectively immersed into the above two PBS solutions (10 mM, 28 mL, containing 0.5% (w/v) Tween 80), and incubated under shaking (100 RPM) at 37° C. for 48 hours. At predetermined intervals, 1 mL of the release medium was collected and replaced with 1 mL of fresh blank medium. As shown in FIG. 12, the concentration of CPT in the dialysate was determined through high performance liquid chromatography (HPLC), with methanol and deionized water (20% to 100%, v/v) as the mobile phase, a flow rate of 1.0 mL/min, a temperature of 25° C., and an absorption wavelength of 370 nm. The concentration of Pt in the dialysate was determined by inductively coupled plasma mass spectrometry (ICP-MS), and the results are shown in FIGS. 13A and 13B.

Conclusion: The dual-drug monomolecular polymer prodrug nano-micelles obtained by chemical polymerization do not release the active drugs in the extracellular microenvironment, but can release the active drugs in response to the intracellular microenvironment.

### 5.3. In Vitro Simulated Drug Release Behavior

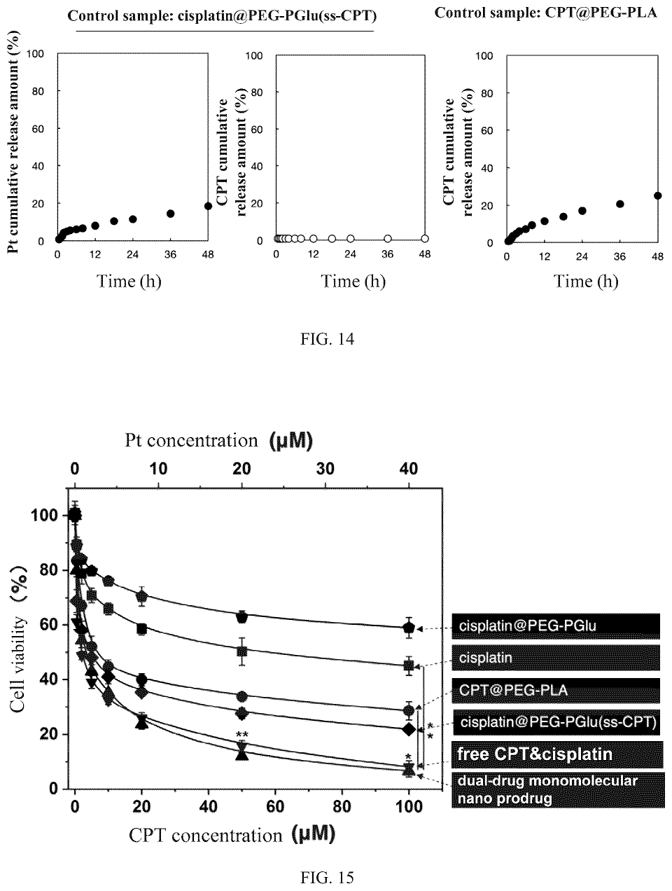

The drug release behavior of the control micelles, cisplatin@PEG-PGlu(ss-CPT) and CPT@PEG-PLA, in the extracellular microenvironment (pH 7.4, GSH (0 mM)) was investigated. The PBS solutions containing the above two nano-formulations (10 mM) were respectively introduced into dialysis bags (MWCO: 10 kDa). The dialysis bags were respectively immersed into the above two PBS solutions (10 mM, 28 mL, containing 0.5% (w/v) Tween 80), and incubated under gentle shaking (100 RPM) at 37° C. for 48 hours. At predetermined intervals, 1 mL of the release medium was collected and replaced with 1 mL of fresh blank medium. The concentration of CPT in the dialysate was determined through HPLC, with methanol and deionized water (20% to 100%, v/v) as the mobile phase, a flow rate of 1.0 mL/min, a temperature of 25° C., and an absorption wavelength of 370 nm. The concentration of Pt in the dialysate was determined by ICP-MS. The results are shown in FIGS. 14A to 14C.

Conclusion. The self-assembled control micelles continuously release the active drug in the extracellular microenvironment.

### Example 6. Cytotoxicity of Dual-Drug Monomolecular Polymer Prodrug Nano-Micelles of the Present Application

Cytotoxicity of cisplatin, cisplatin@PEG-PGlu, cisplatin@PEG-PGlu(ss-CPT), CPT@PEG-PLA, free CPT and cisplatin, and the present monomolecular nano prodrug was respectively evaluated by using the MTT assay. A549 cells at a density of 10⁵ cells/mL were seeded in a 96-well plate (100 μL per well), and incubated in a cell culture incubator at 37° C. and 5% CO₂ for 24 hours. Then, the culture medium was discarded, and the above drug formulations at different concentrations were respectively added into the wells by 100 μL per well. The wells without any drug served as 100% control, and the wells without cells were 0% blank control. After 48 hours of culture, the culture medium was discarded, and 100 μL of MTT solution (0.5 mg/mL) was added to each well and further incubated for 4 hours. Then, the culture medium was carefully removed, and DMSO (100 μL/well) was added to dissolve the blue-purple crystals. After slight shaking, the absorbance at 570 nm (the reference wavelength is 630 nm) was measured using a microplate reader. The formula for cell viability was: Cell viability (%)=[(OD_{sample well}−OD_{blank control})/(OD_{100% control}−OD_{blank control})]100%. The results are shown in FIG. 15.

### Example 7. Pharmacokinetics Study of Dual-Drug Monomolecular Polymer Prodrug Nano-Micelles of the Present Application

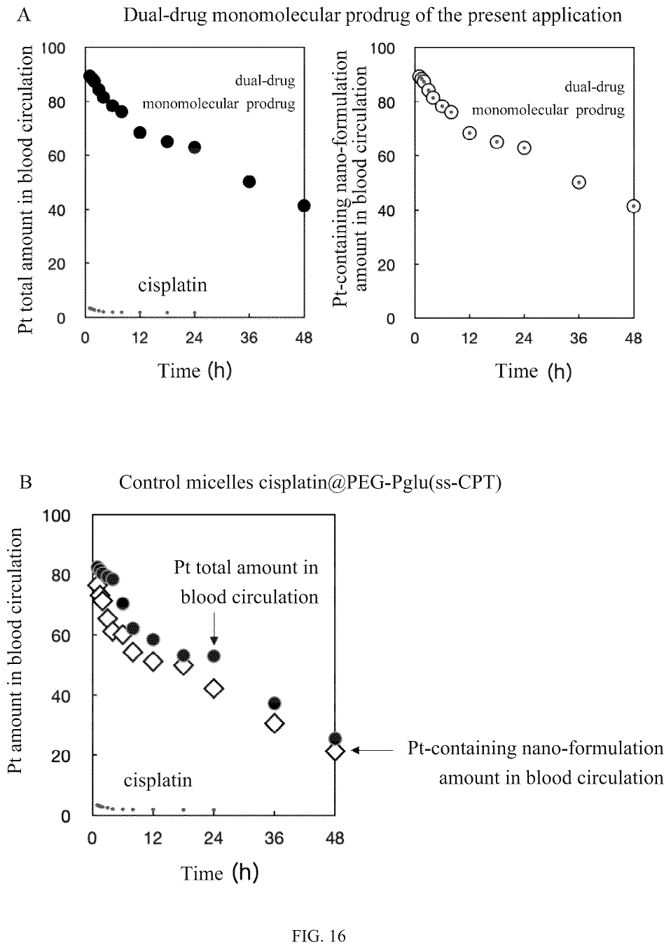

Female Bal b/c mice at the age of 8 weeks were administrated with the drug formulation via tail vein injection at a dose of 200 microliters with camptothecin (CPT) at 5 mg/kg and platinum (Pt) at 1.8 mg/kg. Equivalent amounts of the self-assembled dual-drug nano-formulation cisplatin@PEG-PGlu(ss-CPT) and free cisplatin served as controls. At different time points after intravenous injection, blood was collected and ultracentrifuged at 10,000 G to extract free small-molecule cisplatin and the Pt-containing nano-formulations. The Pt contents were measured using ICP-MS. The results are shown in FIGS. 16A and 16B. The results show that as compared to the free cisplatin control, the blood circulation time of the nano-formulations is significantly prolonged. The monomolecular nano prodrug of the present application does not exhibit early drug leakage in the blood circulation. That is, the total amount of Pt in the blood circulation is consistent with the amount of Pt in the nano-formulation in the blood circulation. In contrast, the self-assembled nano-formulation cisplatin@PEG-PGIu(ss-CPT) exhibits an early drug leakage in the blood circulation. That is, the total amount of Pt in the blood circulation is higher than the amount of Pt in the nano-formulation in the blood circulation.

Upon the experimental results, it can be concluded that as compared to the free cisplatin control, the monomolecular nano prodrug of the present application has significantly prolonged blood circulation time, and will not incur early leakage of free cisplatin in blood circulation. In contrast, as compared to the free cisplatin control, the self-assembled cisplatin@PEG-PGlu(ss-CPT) control also has significantly prolonged blood circulation time, but will incur early leakage of free cisplatin in blood circulation.

### Example 8. Tumor Drug Accumulation Test of Dual-Drug Monomolecular Polymer Prodrug Nano-Micelles of the Present Application

The tumor model used in this example was A549 lung cancer subcutaneous xenograft model. Female Bal b/c nude mice at the age of 8 weeks were administrated with the drug formulation via tail vein injection at a dose of 200 microliters with CPT at 5 mg/kg and Pt at 1.8 mg/kg. Equivalent amounts of cisplatin and the self-assembled dual-drug nano-formulation cisplatin@PEG-PGlu(ss-CPT) served as controls. At different time points after the intravenous injection, the mice were dissected, and the tumor tissues were collected and fully dissolved in concentrated nitric acid. The Pt contents in the tumor tissues were measured using ICP-MS. The results are shown in FIG. 17.

The results show that the concentration of the cisplatin control in the tumor is relatively low, while both the nano-formulations can be significantly enriched in the tumor due to the EPR effect for the nano-formulations. The drug accumulation amount in the tumor gradually increases with time. The accumulation amount of the monomolecular nano prodrug of the present application is significantly higher than that of the self-assembled dual-drug nano-formulation cisplatin@PEG-PGlu(ss-CPT), which may be due to the better colloidal stability of the monomolecular nano prodrug in blood circulation and its performance in preventing early drug leakage.

### Example 9. Evaluation of In Vivo Anti-Tumor Efficacy of Dual-Drug Monomolecular Polymer Prodrug Nano-Micelles of the Present Application

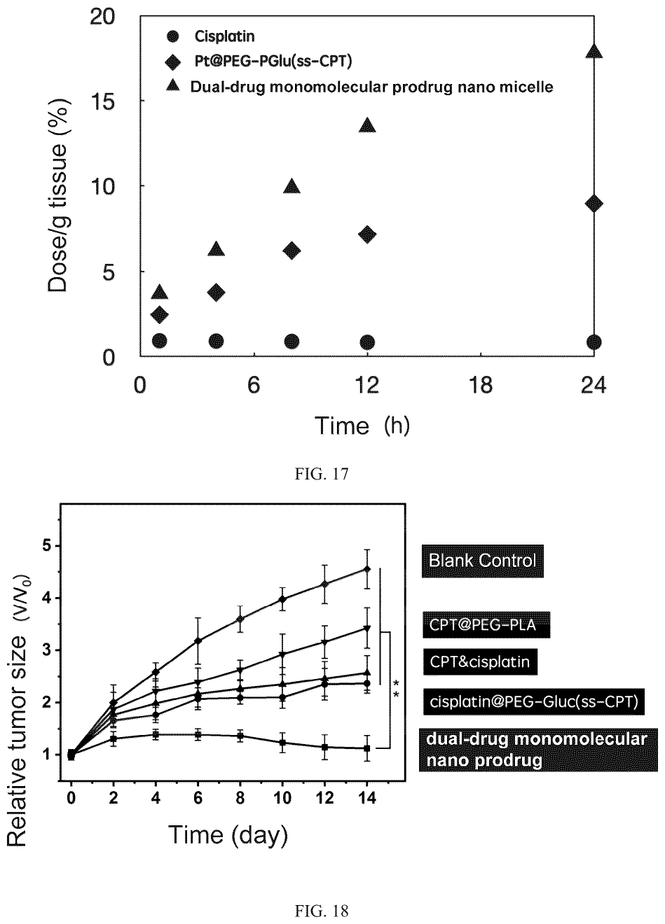

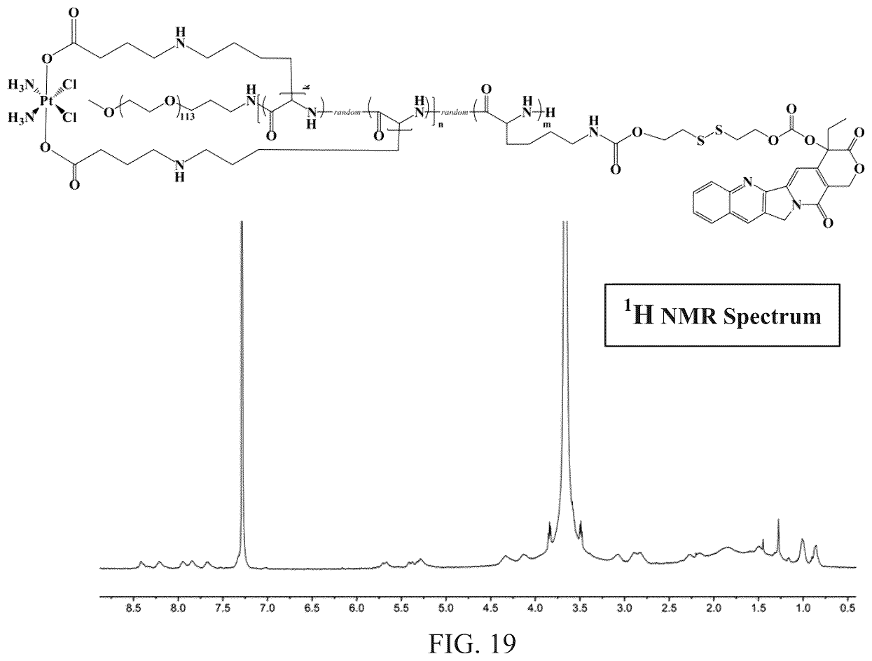

The tumor model used in this example was A549 lung cancer subcutaneous xenograft model. Female Bal b/c nude mice at the age of 8 weeks were administrated with the drug formulation via tail vein injection at a dose of 200 microliters with CPT at 5 mg/kg and Pt at 1.8 mg/kg. The controls were respectively a PBS solution, self-assembled single-drug nano-micelle formulations CPT@PEG-PLA and cisplatin@PEG-PGlu in equivalent amounts, a mixed solution of CPT and small molecule cisplatin in equivalent amount, a self-assembled dual-drug nano-formulation cisplatin@PEG-PGlu(ss-CPT) in equivalent amount. The results are shown in FIG. 18.

The results show that during the anti-tumor test, the mice treated with the monomolecular nano prodrug have a good growth state, and tumor growth is significantly inhibited. The monomolecular nano prodrug significantly outperforms the other controls, indicating that the monomolecular nano prodrug of the present application not only effectively reduces the toxicity of the chemotherapeutic drugs, but also significantly improves the anti-tumor efficacy compared to the conventional self-assembled nano-formulations. Therefore, the monomolecular nano prodrug of the present application has excellent clinical application prospects.

### Example 10. Preparation of Platinum-Based Single-Drug (Cisplatin) Monomolecular Nano-Polymer and Micelles Thereof

### 10.1 Preparation of Platinum-Based Single-Drug Monomolecular Nano-Polymer

Methoxy poly(ethylene glycol) propylamine (MeO-PEG-NH₂, molecular weight 5 kDa, 0.1 g, 0.01 mmol) as a monofunctional hydrophilic polymer was added in benzene (3 mL), stirred to completely dissolve the PEG, then frozen in liquid nitrogen, and dried under vacuum in a cold trap for 6 hours. Then, in a glove box, the dried polyethylene glycol was dissolved in anhydrous DMF (2 mL), and stirred uniformly. NCA-Pt-NCA prepared by the method in Example 2 (0.18 mmol) was dissolved in anhydrous DMF (2 mL), and the resulting solution was added dropwise to the reaction system. The reaction tube was then sealed, taken out from the glove box, and continuously stirred in an oil bath at a temperature of 35° C. for 72 hours. The reaction product was slowly added dropwise into anhydrous diethyl ether to obtain a white precipitate, the supernatant was discarded, and this process was repeated three times to obtain a purified product. The purified product was then vacuum dried in a vacuum pot for 6 hours. The dried solid was dissolved in DMSO (2 mL), introduced into a dialysis bag (MWCO: 100 kDa), dialyzed in ultrapure water for two days (during which water was changed five times), freeze-dried, to obtain a final product.

### 10.2 Characterization of Platinum-Based Single-Drug Monomolecular Nano-Polymer or Micelles Thereof

The characterizations were performed according to the method in Example 4.

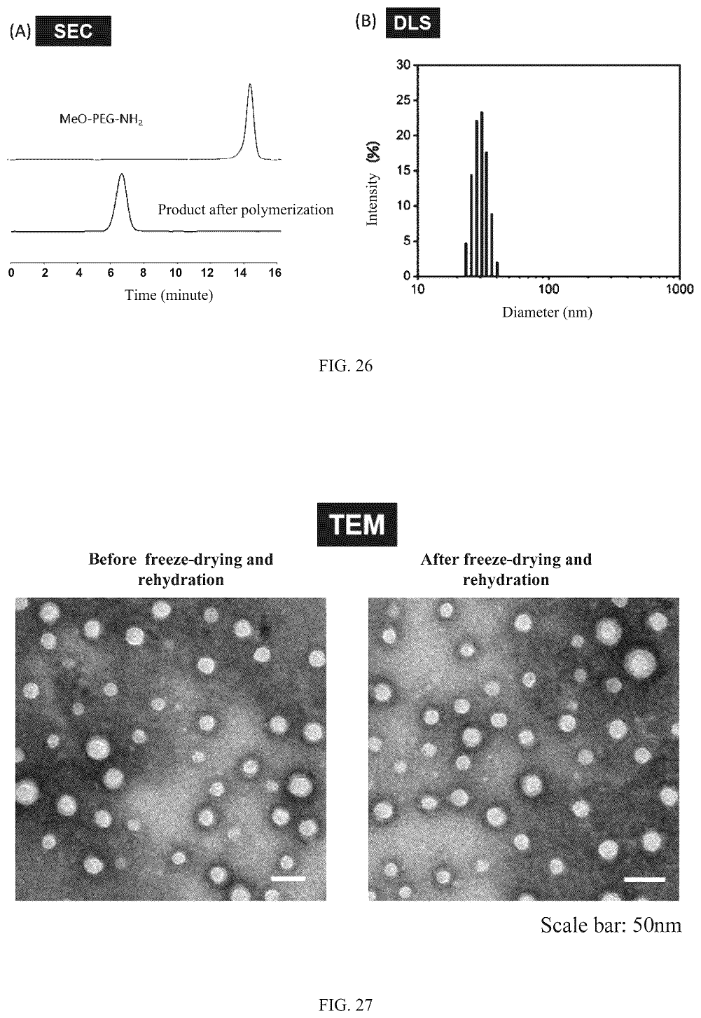

The results of gel permeation chromatography, also called size exclusion chromatography (SEC), are shown in FIG. 26A. Compared to the monofunctional hydrophilic polymer mPEG-NH₂ (5 kDa), the molecular weight of the monomolecular nano-polymer significantly increases after the polymerization, indicating a successful polymerization reaction. The number average molecular weight of the obtained monomolecular nano-polymer is greater than 2000 kDa.

The results of dynamic light scattering (DLS) are shown in FIG. 26B. The product after the polymerization has an average particle size of about 30.4 nm with particle sizes ranging from 23.8 nm to 41.1 nm, and a polydispersity index (PDI) of about 0.05.

The results of transmission electron microscopy (TEM) are shown in FIGS. 27A and 27B. The product after the polymerization is uniform spherical particles, with a diameter less than 50 nm, and an average diameter of about 30 nm, which is substantially consistent with the DLS results. After freeze-drying and rehydration, the size and morphology of the particles are still stable.

It can be seen that the cisplatin monomolecular polymer nano-particles with a uniform size can be obtained through chemical polymerization, and can be used as a prodrug.

### Example 11. Preparation of Platinum-Based Single-Drug (DACHPt) Monomolecular Nano-Polymer and Micelles Thereof

### 11.1 Preparation of NCA-Pt-NCA (i.e., NCA-DACHPt-NCA)

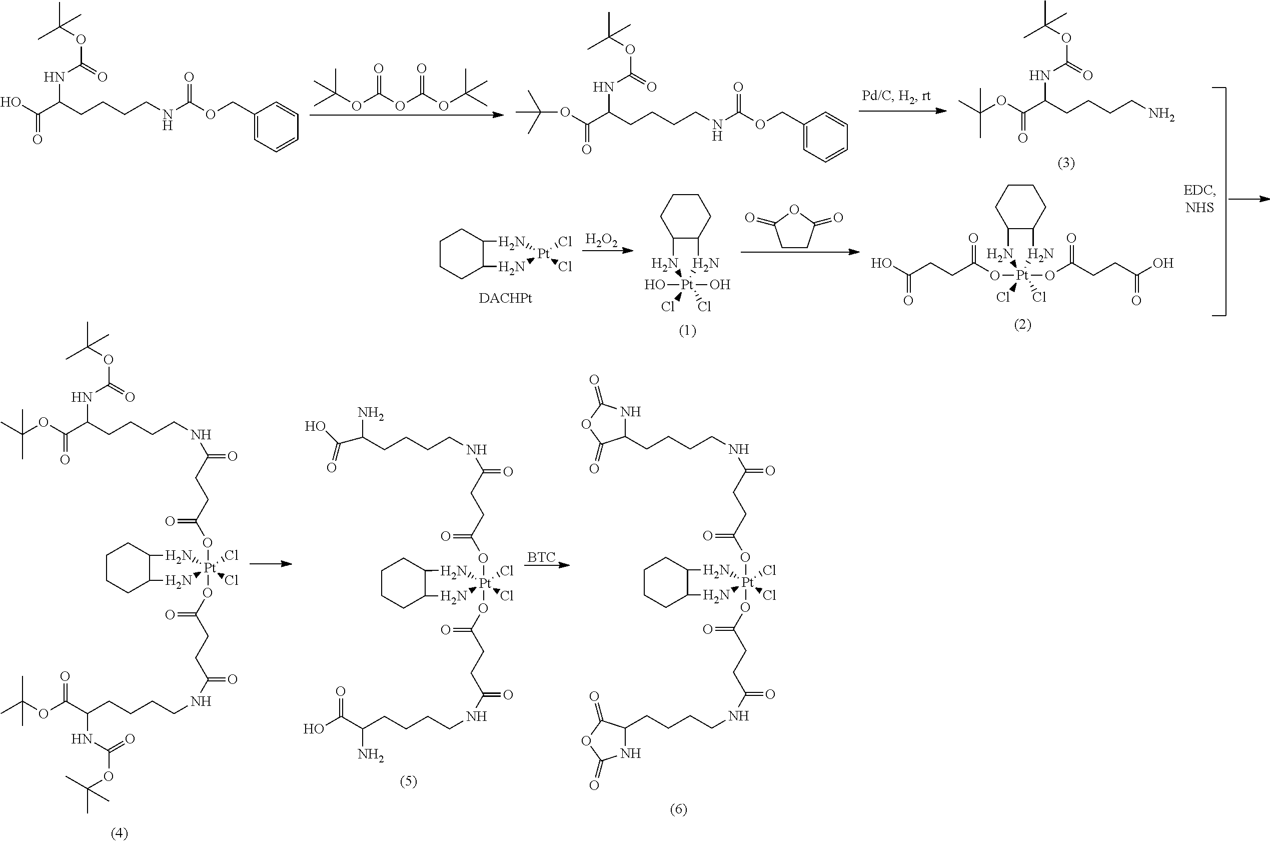

Dichloro (1,2-diaminocyclohexane) platinum (II) (DACHPt, 3.8 g, 10 mmol) was dispersed in distilled water, and 30% H₂O₂ (60 ml) was added. The mixture was stirred in the dark at 70° C. for 5 hours until becoming clear, cooled to room temperature, and then placed in a refrigerator at 4° C. for recrystallization. The formed crystals were filtered, and the filter cake was washed with ice water, ethanol, and diethyl ether sequentially, then dried to obtain compound (1) crystals. Compound (1) (0.414 mg, 1.0 mmol) and succinic anhydride (0.62 g, 6.15 mmol) were dissolved in DMF (20 ml), and the mixture was stirred overnight in the dark at 70° C., followed by distillation under reduced pressure to remove the solvent, washed with methanol and acetone, and then dried to obtain compound (2).

Compound (2) (0.1 mmol) was dissolved in anhydrous DMF (5.0 mL), mixed with a solution of EDC (0.15 mmol) and DMAP (0.05 mmol) in anhydrous DMF (5.0 mL), and then reacted for 30 minutes. After that, a solution of compound (3) (0.1 mmol) in anhydrous DMF (5.0 mL) was added and mixed. The mixed solution was stirred at room temperature for 6 hours, and then separated by column chromatography to obtain a light yellow solid (4).

Compound (4) (0.1 mmol) was dissolved in DMF (2.0 mL), mixed with trifluoroacetic acid (2.0 mL), and reacted at room temperature for 2 hours. The reacted solution was distilled under reduced pressure to remove the solvent, then washed with methanol and acetone sequentially, and dried to obtain light yellow compound (5).

The compound (5) (0.1 mmol) was dissolved in DMF (2.0 mL), mixed with a solution of triphosgene (0.2 mmol) in DMF (2.0 mL), and then reacted at room temperature for 2 hours. The reacted solution was distilled under reduced pressure to remove the solvent, and dissolved in tetrahydrofuran at 60° C., and then an appropriate amount of n-hexane was added. The solution was placed in a refrigerator at 4° C. for crystallization, and white needle-like crystals (6) NCA-Pt-NCA (specifically NCA-DACHPt-NCA) were collected.

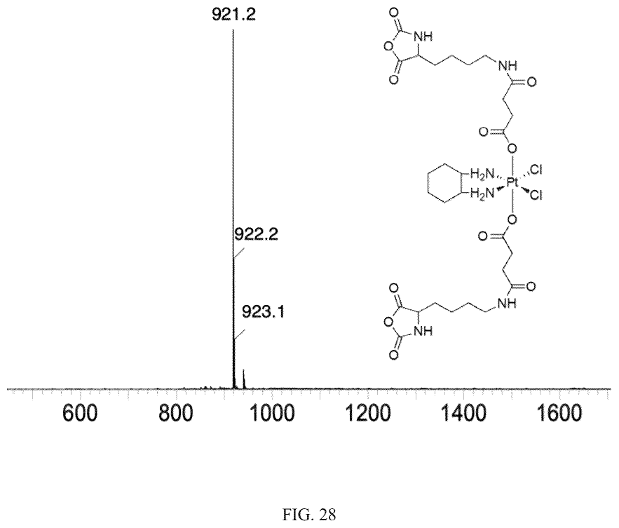

Mass spectrum of NCA-DACHPt-NCA is shown in FIG. 28.

In other examples, unless otherwise specified, the ¹H NMR test can be conducted according to the method in this example.

### 11.2 Preparation of Platinum-Based Single-Drug Monomolecular Nano-Polymer P102

Methoxy poly(ethylene glycol) propylamine (mPEG-CH₂CH₂CH₂NH₂, abbreviated as mPEG-NH₂, 5 kDa, 0.05 g, 0.01 mmol) was added in benzene (3 mL), magnetically stirred to completely dissolve the PEG, then frozen in liquid nitrogen, and dried under vacuum in a cold trap for 6 hours. After transferred into a water-free and oxygen-free glove box, the dried polyethylene glycol was dissolved in anhydrous DMF (2 mL), and magnetically stirred uniformly. NCA-DACHPt-NCA (0.4 mmol) was completely dissolved in anhydrous DMF (2 mL), and the resulting solution was slowly added dropwise to the reaction system containing the mPEG-NH₂. The generated carbon dioxide was collected using a balloon. The reaction system was then sealed, taken out from the glove box, and continuously stirred in an oil bath at a temperature of 35° C. for 72 hours. The reaction product was slowly added dropwise into anhydrous diethyl ether to obtain a white precipitate, and the supernatant was discarded, and this process was repeated three times to obtain a purified product. The purified product was then dried under vacuum in a vacuum pot for 6 hours. The dried solid was dissolved in DMSO (10 mL), introduced into a dialysis bag (MWCO: 100 kDa), dialyzed in ultrapure water for two days (during which water was changed five times), freeze-dried, to obtain a final product P102.

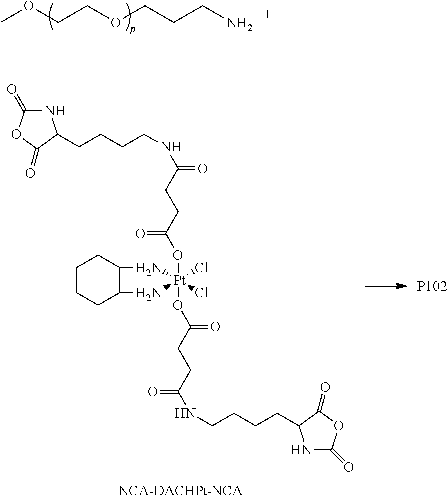

### 11.3 Characterization of Platinum-Based Single-Drug Monomolecular Nano-Polymer and Micelles Thereof

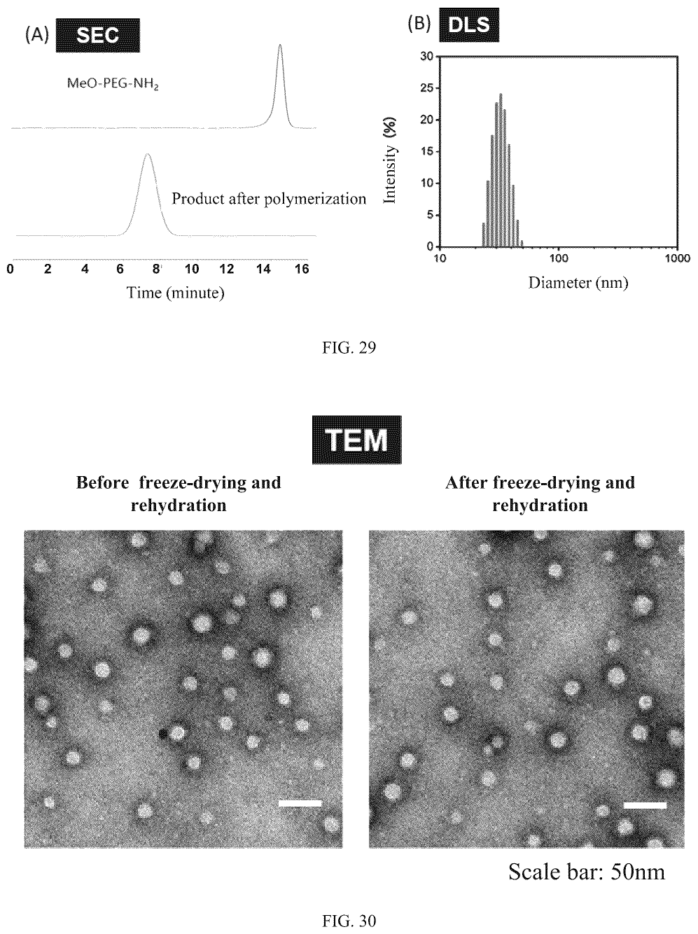

The results of gel permeation chromatography, also called as SEC, are shown in FIG. 29A. Compared to the monofunctional hydrophilic polymer mPEG-NH₂ (5 kDa), the molecular weight of the monomolecular nano-polymer significantly increased after the polymerization, indicating a successful polymerization reaction. The number average molecular weight of the obtained monomolecular nano-polymer was greater than 2000 kDa.

The results of dynamic light scattering (DLS) are shown in FIG. 29B. The product after the polymerization has an average particle size of about 34 nm with particle sizes ranging from 24.2 nm to 43.5 nm, and a polydispersity index (PDI) of about 0.05.

The results of transmission electron microscopy (TEM) are shown in FIGS. 30A and 30B. The product after the polymerization are uniform spherical particles, with a diameter less than 50 nm, and an average diameter of about 30 nm, which are substantially consistent with the DLS results. A comparison before and after the freeze-drying and rehydration shows that the size and morphology of the particles are stable.

It can be seen that the DACHPt monomolecular polymer nano-particles with a uniform size can be obtained through chemical polymerization, and can be used as a prodrug.

### Example 12. Preparation of Dual-Drug (Cisplatin+Paclitaxel) Monomolecular Nano-Polymer and Micelles Thereof

### 12.1 Preparation of PTX-ss-OH

Paclitaxel (PTX, 854 mg, 1.0 mmol) was dispersed in anhydrous dichloromethane (200 mL). In an ice bath and under nitrogen gas protection, a solution of triphosgene (297 mg, 1.0 mmol) in anhydrous dichloromethane (5 mL) was added, and the mixture was stirred in the ice bath for 30 minutes to thoroughly dissolve the paclitaxel. Then, DMAP (488 mg, 4.0 mmol) dissolved in anhydrous dichloromethane (10 mL) was added. The stirring was continued in the ice bath for 1 hour, and then in the dark at room temperature for another hour. 2-hydroxyethyl disulfide (17 mL, 15 mmol) dissolved in anhydrous THF (15 mL) was added under nitrogen gas protection. After mixing well, the reaction solution was stirred in the dark at room temperature for 24 hours. After completion of the reaction, additional 50 mL of dichloromethane was added. The mixed solution was washed three times each with 0.1 M HCl aqueous solution, saturated NaCl solution, and water sequentially. The organic phase was collected, dried with anhydrous Na₂SO₄, and distilled under reduced pressure by using a rotary evaporator to remove the organic solvent. The resulting yellow solid was purified through a silica gel column using dichloromethane/methanol as an eluent, followed by rotary evaporation to obtain a light yellow product PTX-ss-OH.

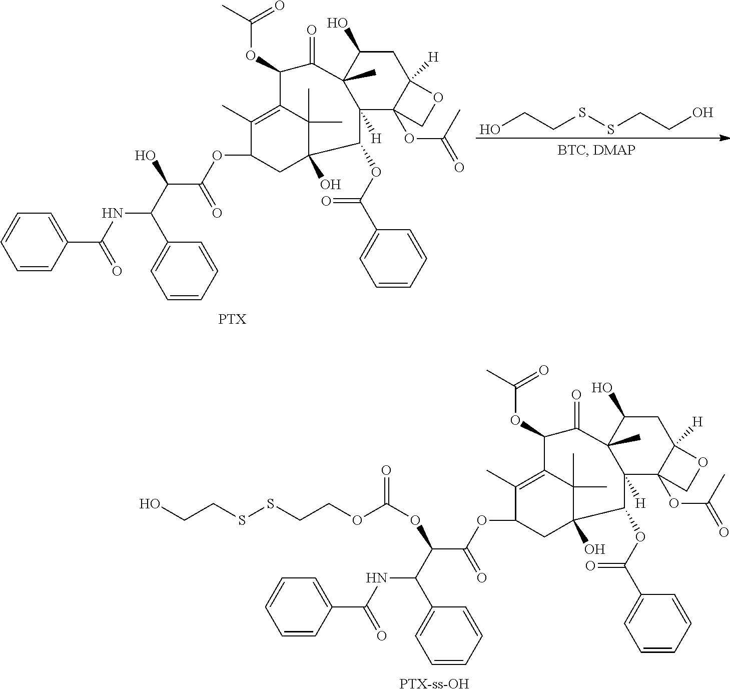

### 12.2 Preparation of PTX-ss-NCA

By using PTX-ss-OH instead of CPT-ss-OH as a raw material, PTX-ss-NCA was prepared according to the methods in sections 1.4 to 1.6 of Example 1.

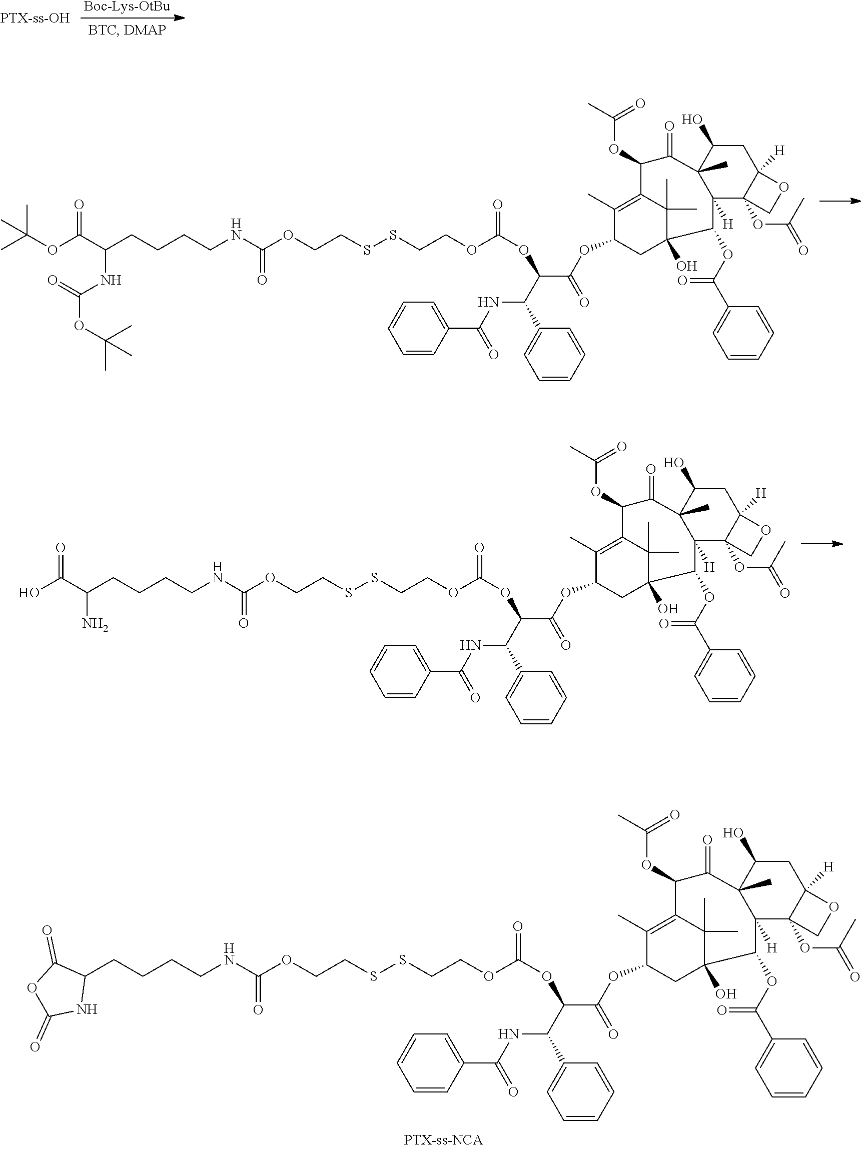

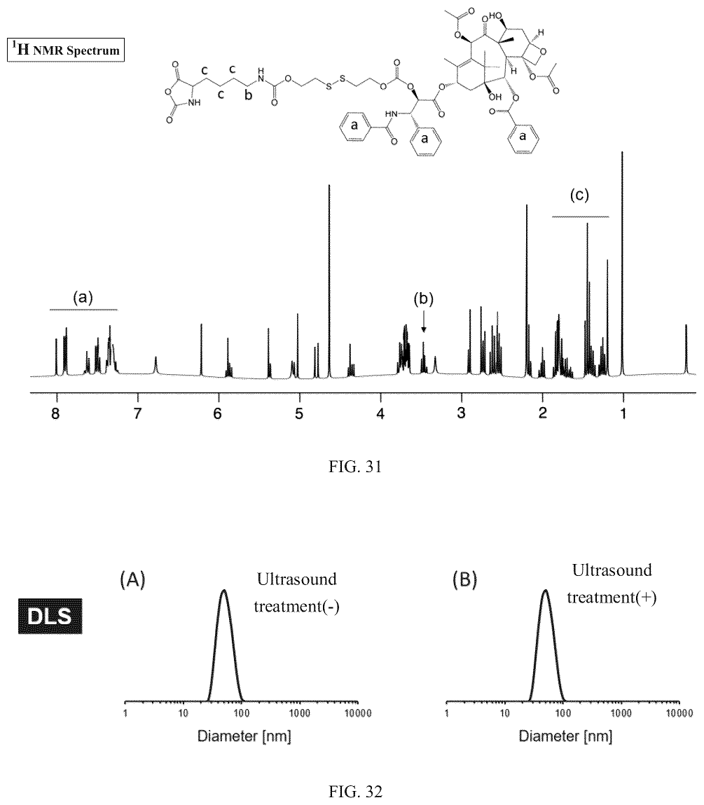

¹H NMR spectrum of PTX-ss-NCA is shown in FIG. 31. Three peaks between 7.2 ppm and 8.1 ppm correspond to three benzene rings of paclitaxel. The peak at 3.4 ppm indicates successful bonding between the paclitaxel derivative and NCA-Lysine. The peaks between 1.2 ppm and 1.9 ppm correspond to the n-butyl of the side chain of lysine.

### 12.3 Preparation of Dual-Drug Monomolecular Nano-Polymer P103

Methoxy poly(ethylene glycol) propylamine (MeO-PEG-NH₂, molecular weight 5 kDa, 0.2 g, 0.01 mmol) was added in benzene (3 mL), magnetically stirred to completely dissolve the PEG, then frozen in liquid nitrogen, and vacuum dried in a cold trap for 6 hours. After transferred into a water-free and oxygen-free glove box, the dried polyethylene glycol was dissolved in anhydrous DMF (2 mL), and stirred uniformly. NCA-Pt-NCA (0.2 mmol) prepared by the method in Example 2 and PTX-ss-NCA (0.2 mmol) were completely dissolved in anhydrous DMF (30 mL), and the resulting solution was slowly added dropwise to the reaction system containing the MeO-PEG-NH₂. The generated carbon dioxide was collected using a balloon. The reaction system was then sealed, taken out from the glove box, and continuously stirred in an oil bath at a temperature of 35° C. for 72 hours. The reaction product was slowly added dropwise into anhydrous diethyl ether to obtain a white precipitate, and the supernatant was discarded, and this process was repeated three times to obtain a purified product. The purified product was then dried under vacuum in a vacuum pot for 6 hours. The dried solid was dissolved in DMSO (12 mL), introduced into a dialysis bag (MWCO: 100 kDa), dialyzed in ultrapure water for two days (during which water was changed five times), freeze-dried, to obtain a final product P103.

### 12.4 Characterization of Dual-Drug Monomolecular Nano-Polymer and Micelles Thereof

The characterizations were performed according to the method in Example 4.

The dynamic light scattering (DLS) results without and with ultrasonic treatment are shown in FIGS. 32A and 32B respectively. The ultrasonic parameters were 1.0 MHz and 9.9 W, and the ultrasonic time period was 60 minutes. The product after the polymerization has an average diameter of about 46.4 nm with particle sizes ranging from 34.6 nm to 61.8 nm, indicating successful polymerization. The product can withstand ultrasonic treatment, with its particle size unchanged before and after the ultrasonic treatment.

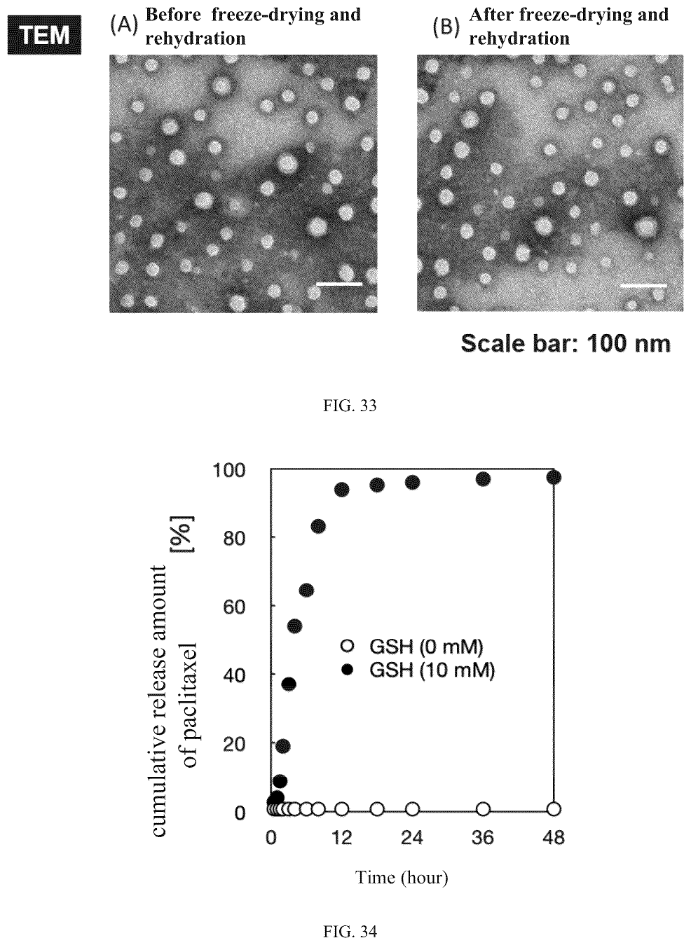

In the transmission electron microscopy (TEM) test, the dialyzed solution was freeze-dried and rehydrated (1 mg/mL) three times. The results before and after the rehydration are shown in FIGS. 33A and 33B respectively. The product after the polymerization is uniform spherical particles, with an average diameter of about 40 nm, which is substantially consistent with the DLS result. After the freeze-drying and rehydration, the size and morphology of the particles are still stable.

It can be seen that the monomolecular polymer nano-particles with a uniform size and a stable structure can be obtained through chemical polymerization.

### 12.5 Study of Drug Release Behavior

The drug release behavior of the dual-drug (cisplatin+PTX) monomolecular nano-polymer was investigated under different conditions: (1) extracellular microenvironment: pH 7.4, GSH (0 mM); and (2) intracellular microenvironment: pH 7.4, GSH (10 mM). The PBS solution containing the above nano-formulation (10 mM) was introduced in dialysis bags (MWCO: 10,000). The dialysis bags were respectively immersed into the above two PBS solutions (10 mM, 28 mL, containing 0.5% (w/v) Tween 80), and incubated under shaking (100 RPM) at 37° C. for 48 hours. At predetermined intervals, 1 mL of the release medium was collected and replaced with 1 mL of fresh blank medium. The concentration of PTX in the dialysate was determined through HPLC, with methanol and deionized water (20% to 100%, v/v) as the mobile phase, a flow rate of 1.0 mL/min, a temperature of 25° C., and an absorption wavelength of 270 nm.

The results are shown in FIG. 34. It can be seen from the results that the dual-drug (cisplatin+PTX) monomolecular nano-polymer obtained by chemical polymerization as a prodrug does not release the active drug in the extracellular microenvironment (0 mM GSH), but can release the active drug in response to the intracellular microenvironment (10 mM GSH).

### Example 13. Preparation of Dual-Drug (Cisplatin+R848) Monomolecular Nano-Polymer and Micelles Thereof

### 13.1 Preparation of R848-ss-OH

By using R848 as a raw material, R848-ss-OH was prepared according to the method in section 1.3 of Example 1.

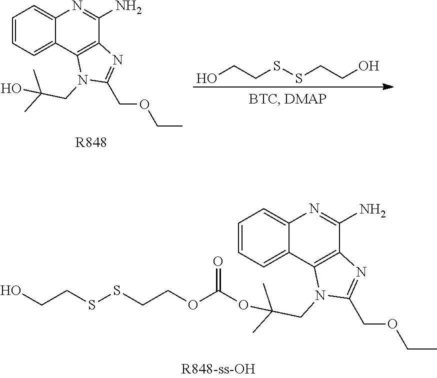

### 13.2 Preparation of R848-ss-NCA

By using R848-ss-OH instead of CPT-ss-OH as a raw material, R848-ss-NCA was prepared according to the methods in sections 1.4 to 1.6 of Example 1.

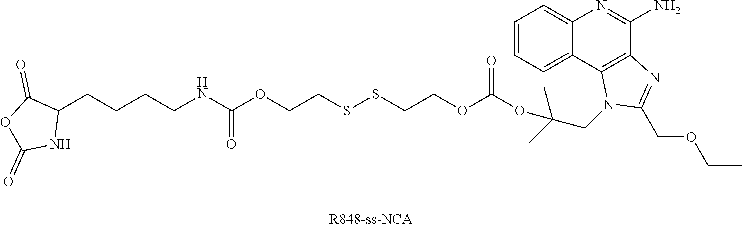 [SMILES: CCOCc1nc2c(N)nc3ccccc3c2n1CC(C)(C)OC(=O)OCCSSCCOC(=O)NCCCCC1NC(=O)OC1=O]

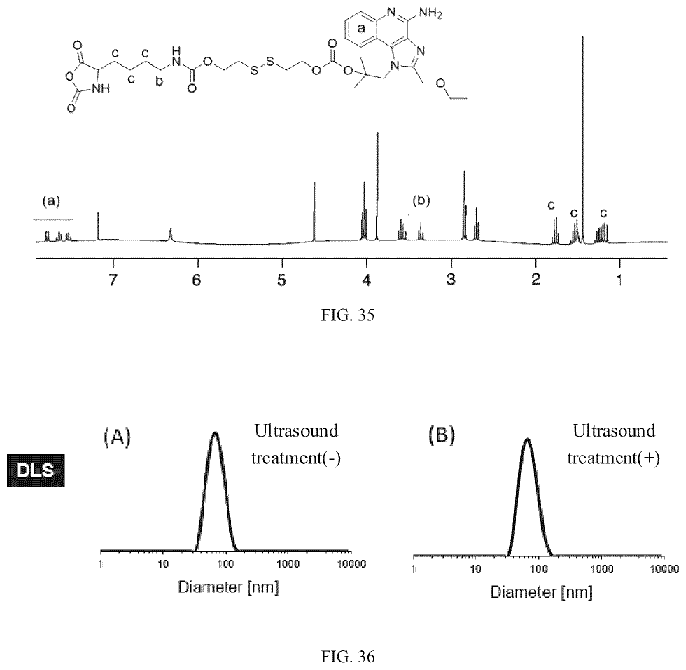

¹H NMR spectrum of R848-ss-NCA is shown in FIG. 35. The peaks in the range above 7.2 ppm correspond to the biphenyl of R848. The peak at 3.4 ppm indicates successful bonding between R848 derivative and NCA-Lysine. The peaks between 1.2 ppm and 1.9 ppm (except for the single high peak at 1.45 ppm) correspond to the n-butyl of the side chain of lysine.

### 13.3 Preparation of Dual-Drug Monomolecular Nano-Polymer P104

Methoxy poly(ethylene glycol) propylamine (MeO-PEG-NH₂, molecular weight 5 kDa, 0.1 g, 0.01 mmol) was added in benzene (3 mL), magnetically stirred to completely dissolve the PEG, then frozen in liquid nitrogen, and vacuum dried in a cold trap for 6 hours. Then in a glove box, the dried polyethylene glycol was dissolved in anhydrous DMF (2 mL), and stirred uniformly. NCA-Pt-NCA (0.2 mmol) prepared by the method in Example 2 and R848-ss-NCA (0.2 mmol) were dissolved in anhydrous DMF (30 mL), and the resulting solution was slowly added dropwise to the reaction system containing the MeO-PEG-NH₂. The generated carbon dioxide was collected using a balloon. The reaction system was then sealed, taken out from the glove box, and continuously stirred in an oil bath at a temperature of 35° C. for 72 hours. The reaction product was slowly added dropwise into anhydrous diethyl ether to obtain a white precipitate, and the supernatant was discarded, and this process was repeated three times to obtain a purified product. The purified product was then dried under vacuum in a vacuum pot for 6 hours. The dried solid was dissolved in DMSO (12 mL), introduced into a dialysis bag (MWCO: 100 kDa), dialyzed in ultrapure water for two days (during which water was changed five times), freeze-dried, to obtain a final product P104.

### 13.4 Characterization of Dual-Drug Monomolecular Nano-Polymer and Micelles Thereof

The characterizations were performed according to the method in Example 4.

The dynamic light scattering (DLS) results without and with ultrasonic treatment are shown in FIGS. 36A and 36B respectively. The ultrasonic parameters were 1.0 MHz and 9.9 W, and the ultrasonic time period was 60 minutes. The product after the polymerization has an average diameter of about 44.9 nm with particle sizes ranging from 32.8 nm to 66.2 nm, indicating successful polymerization. The product can withstand ultrasonic treatment, with its particle size unchanged before and after the ultrasonic treatment.

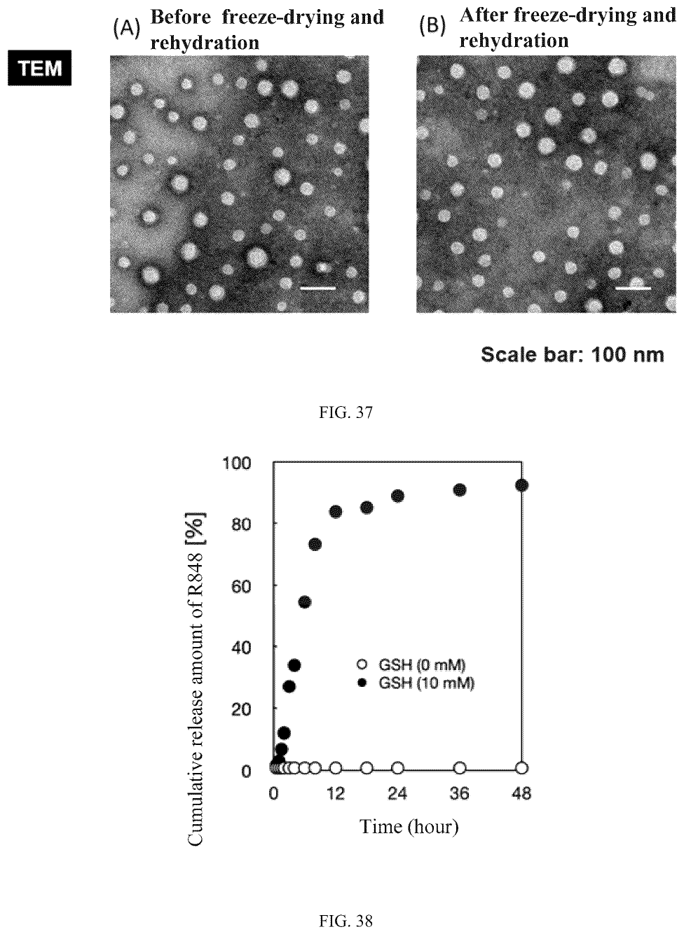

In the transmission electron microscopy (TEM) test, the dialyzed solution was freeze-dried and rehydrated (1 mg/mL) three times. The results before and after the rehydration are shown in FIGS. 37A and 37B respectively. The product after the polymerization is uniform spherical particles, with an average diameter of about 40 nm, which is substantially consistent with the DLS result. After the freeze-drying and rehydration, the size and morphology of the particles are still stable.

It can be seen that the monomolecular polymer nano-particles with a uniform size and a stable structure can be obtained through chemical polymerization.

### 13.5 Study of Drug Release Behavior

The drug release behavior of the dual-drug (cisplatin+R848) monomolecular nano-polymer was investigated under different conditions: (1) extracellular microenvironment: pH 7.4, GSH (0 mM), and (2) intracellular microenvironment: pH 7.4, GSH (10 mM). The PBS solution containing the above nano-formulation (10 mM) was introduced into dialysis bags (MWCO: 10,000). The dialysis bags were respectively immersed into the above two PBS solutions (10 mM, 28 mL, containing 0.5% (w/v) Tween 80), and incubated under shaking (100 RPM) at 37° C. for 48 hours. At predetermined intervals, 1 mL of release medium was collected and replaced with 1 mL of fresh blank medium. The concentration of R848 in the dialysate was determined through HPLC, with methanol and deionized water (20% to 100%, v/v) as the mobile phase, a flow rate of 1.0 mL/min, a temperature of 25° C., and an absorption wavelength of 320 nm.

The results are shown in FIG. 38. It can be seen from the results that the dual-drug (cisplatin+R848) monomolecular nano-polymer obtained by chemical polymerization as a prodrug does not release the active drug in the extracellular microenvironment (0 mM GSH), but can release the active drug in response to the intracellular microenvironment (10 mM GSH).

### Example 14. Preparation of Dual-Drug (Cisplatin+MMAE) Monomolecular Nano-Polymer and Micelles Thereof

### 14.1 Preparation of MMAE-ss-OH

By using MMAE as a raw material, MMAE-ss-OH was prepared according to the method in section 1.3 of Example 1.

MMAE (718 mg, 1.0 mmol) was dissolved in anhydrous dichloromethane (20 mL). In an ice bath and under nitrogen gas protection, a solution of triphosgene (297 mg, 1.0 mmol) in anhydrous dichloromethane (5 mL) was added, and the mixture was stirred in the ice bath for 30 minutes to thoroughly dissolve the MMAE. Then, DMAP (488 mg, 4.0 mmol) dissolved in anhydrous dichloromethane (10 mL) was added. The stirring was continued in the ice bath for 1 hour, and then in the dark at room temperature for another hour. 2-hydroxyethyl disulfide (17 mL, 15 mmol) dissolved in anhydrous THF (15 mL) was added under nitrogen gas protection. After mixing well, the reaction solution was stirred in the dark at room temperature for 24 hours. After completion of the reaction, additional 50 mL of dichloromethane was added. The mixed solution was washed three times each with 0.1 M HCl aqueous solution, saturated NaCl, and water sequentially. The organic phase was collected, dried with anhydrous Na₂SO₄, and distilled under reduced pressure by using a rotary evaporator to remove the organic solvent. The resulting yellow solid was purified through a silica gel column using dichloromethane/methanol as an eluent, followed by rotary evaporation to obtain a light yellow product MMAE-ss-OH.

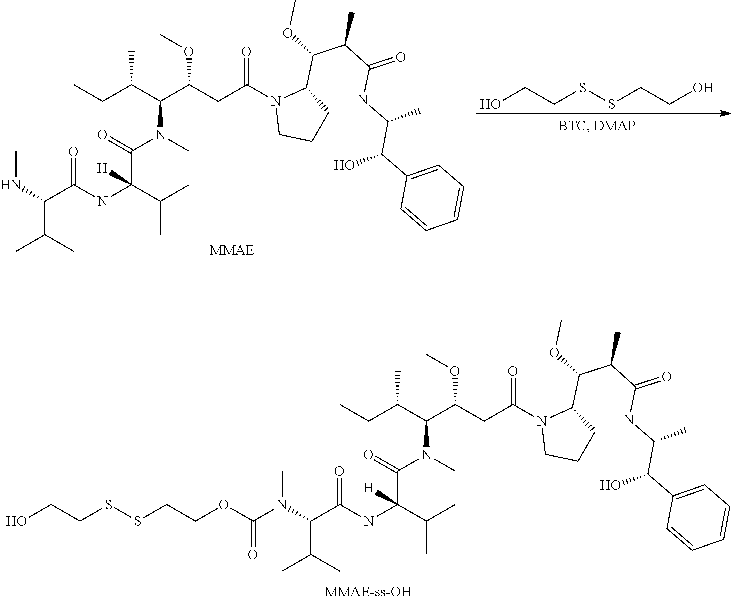 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C ; OCCSSCCO ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCCSSCCO)C(C)C]

### 14.2 Preparation of MMAE-ss-NCA

By using MMAE-ss-OH instead of CPT-ss-OH as a raw material, MMAE-ss-NCA was prepared according to the methods in sections 1.4 to 1.6 of Example 1.

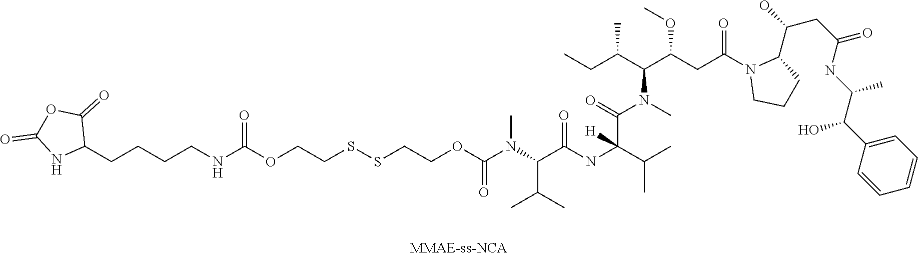 [SMILES: CC[C@H](C)[C@@H]([C@@H](CC(=O)N1CCC[C@H]1[C@H](O)CC(=O)N[C@H](C)[C@@H](O)c1ccccc1)OC)N(C)C(=O)[C@@H](NC(=O)[C@H](C(C)C)N(C)C(=O)OCCSSCCOC(=O)NCCCCC1NC(=O)OC1=O)C(C)C]

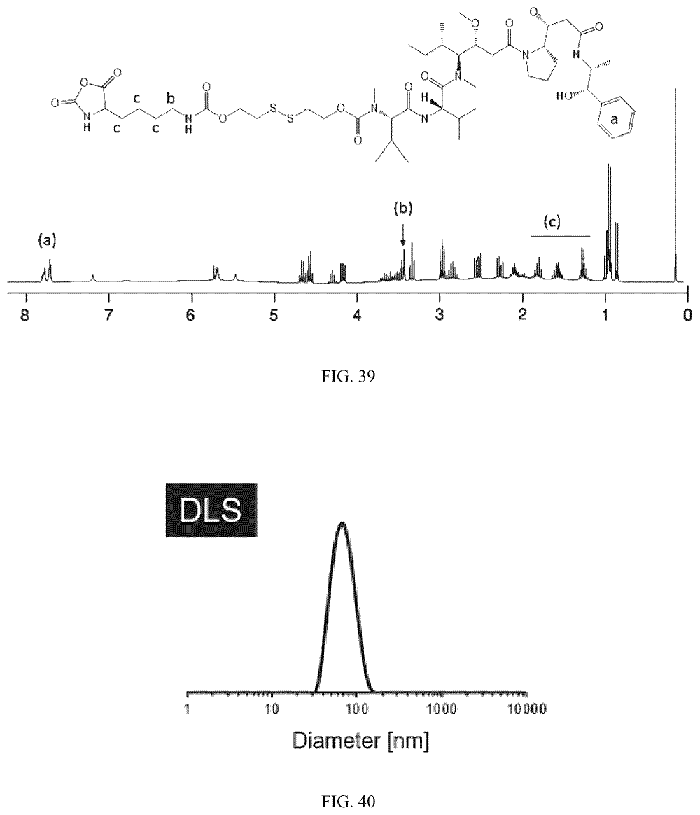

¹H NMR spectrum of MMAE-ss-NCA is shown in FIG. 39. The peaks in the range above 7.6 ppm correspond to the biphenyl of MMAE. The peak at 3.4 ppm indicates successful bonding between the MMAE derivative and NCA-Lysine. The peaks between 1.2 ppm and 1.9 ppm correspond to the n-butyl of the side chain of lysine.

### 14.3 Preparation of Dual-Drug (Cisplatin+MMAE) Monomolecular Nano-Polymer P105

Methoxy poly(ethylene glycol) propylamine (MeO-PEG-NH₂, molecular weight 5 kDa, 0.1 g, 0.01 mmol) was added in benzene (3 mL), magnetically stirred to completely dissolve the PEG, then frozen in liquid nitrogen, and vacuum dried in a cold trap for 6 hours. After transferred into a water-free and oxygen-free glove box, the dried polyethylene glycol was dissolved in anhydrous DMF (2 mL), and magnetically stirred uniformly. NCA-Pt-NCA (0.2 mmol) prepared by the method in Example 2 and MMAE-ss-NCA (0.2 mmol) were dissolved in anhydrous DMF (30 mL), and the resulting solution was slowly added dropwise to the reaction system containing the MeO-PEG-NH₂. The generated carbon dioxide was collected using a balloon. The reaction system was then sealed, taken out from the glove box, and continuously stirred in an oil bath at a temperature of 35° C. for 72 hours. The reaction product was slowly added dropwise into anhydrous diethyl ether to obtain a white precipitate, and the supernatant was discarded, and this process was repeated three times to obtain a purified product. The purified product was then dried under vacuum in a vacuum pot for 6 hours. The dried solid was dissolved in DMSO (12 mL), introduced into a dialysis bag (MWCO: 100 kDa), dialyzed in ultrapure water for two days (during which water was changed five times), freeze-dried, to obtain a final product P105.

### 14.4 Characterization of Dual-Drug (Cisplatin+MMAE) Monomolecular Nano-Polymer and Micelles Thereof

The characterizations were performed according to the method in Example 4.

The dynamic light scattering (DLS) results without ultrasonic treatment is shown in FIG. 40. The product after the polymerization has an average diameter of about 43.9 nm with particle sizes ranging from 30.5 nm to 58.4 nm, indicating successful polymerization.

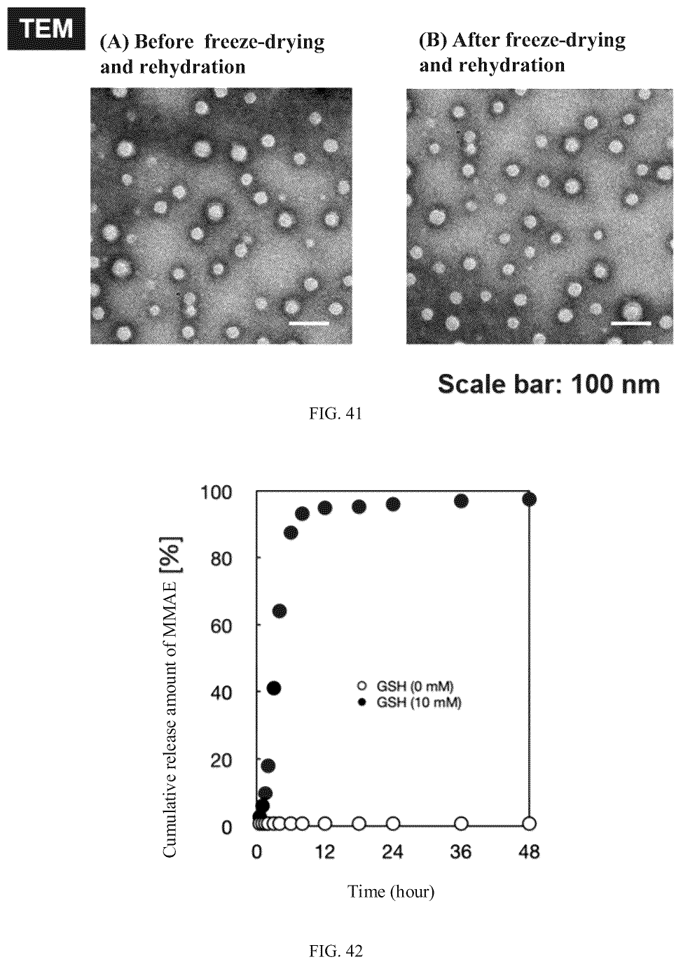

In the transmission electron microscopy (TEM) test, the dialyzed solution was freeze-dried and rehydrated (1 mg/mL) three times. The results before and after the rehydration are shown in FIGS. 41A and 41B respectively. The product after the polymerization is uniform spherical particles, with an average diameter of about 40 nm, which is substantially consistent with the DLS result. After the freeze-drying and rehydration, the size and morphology of the particles are still stable.

It can be seen that the monomolecular polymer nano-particles with a uniform size and a stable structure can be obtained through chemical polymerization.

### 14.5 Study of Drug Release Behavior

The drug release behavior of the dual-drug (cisplatin+MMAE) monomolecular nano-polymer was investigated under different conditions: (1) extracellular microenvironment: pH 7.4, GSH (0 mM); and (2) intracellular microenvironment: pH 7.4, GSH (10 mM). The PBS solution containing the above nano-formulation (10 mM) was introduced into dialysis bags (MWCO: 10,000). The dialysis bags were respectively immersed into the above two PBS solutions (10 mM, 28 mL, containing 0.5% (w/v) Tween 80), and incubated under shaking (100 RPM) at 37° C. for 48 hours. At predetermined intervals, 1 mL of the release medium was collected and replaced with 1 mL of fresh blank medium. The concentration of MMAE in the dialysate was determined through HPLC, with methanol and deionized water (20% to 100%, v/v) as the mobile phase, a flow rate of 1.0 mL/min, a temperature of 25° C., and an absorption wavelength of 280 nm.

The results are shown in FIG. 42. It can be seen from the results that the dual-drug (cisplatin+MMAE) monomolecular nano-polymer obtained by chemical polymerization as a prodrug does not release the active drug in the extracellular microenvironment (0 mM GSH), but can release the active drug in response to the intracellular microenvironment (10 mM GSH).

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the present application.

The above-described embodiments are only several implementations of the present application, and the descriptions are relatively specific and detailed, but they should not be construed as limiting the scope of the present application. It should be understood by those of ordinary skill in the art that various modifications and improvements can be made without departing from the concept of the present application, and all fall within the protection scope of the present application. In addition, it should be understood that, after reading the above teachings of the present application, those of ordinary skill in the art can make various changes or modifications to the present application, and the equivalent forms obtained are also within the protection scope of the present application. It should also be understood that the technical solutions obtained by those of ordinary skill in the art through logical analysis, reasoning or limited tests on the basis of the technical solutions provided in the present application are within the protection scope of the appended claims of the present application. Therefore, the protection scope of the present application shall be defined by the appended claims, and the specification and drawings can be used to interpret the contents of the claims.

1-47. (canceled)

48. A drug-loaded monomolecular nano-polymer, comprising:
a plurality of poly(amino acid) chains,
a plurality of divalent linking groups, and
at least one hydrophilic polymer chain;
wherein the plurality of poly(amino acid) chains forms a non-linear backbone through interchain covalent bonding with the plurality of divalent linking groups, an end of at least one of the poly(amino acid) chains is connected with the hydrophilic polymer chain, a linear backbone of each of the divalent linking groups comprises a platinum-based drug unit comprising a platinum atom, and the platinum-based drug unit is a residue of either a platinum-based drug active ingredient or a prodrug of the platinum-based drug active ingredient.

49. The drug-loaded monomolecular nano-polymer according to claim 48, comprising at least one second drug unit, wherein at least one of the plurality of poly(amino acid) chains is grafted with the second drug unit at a side group, and the second drug unit is a residue of either an anti-tumor drug active ingredient or a prodrug of the anti-tumor drug active ingredient;
the second drug unit at each occurrence is independently connected to a corresponding amino acid unit of the non-linear backbone through a responsive linking group, and the responsive linking group is capable of having bond cleavage under external stimulation.

50. The drug-loaded monomolecular nano-polymer according to claim 49, wherein a main chain of each of the plurality of poly(amino acid) chains is formed by sequential bonding a plurality of 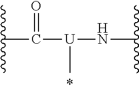
via —C(═O)—NH— bonds, in each 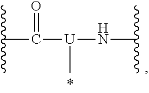
U is independently a carbon-centered trivalent group, and each “*” terminal is independently connected to one of the plurality of divalent linking groups, or to a hydrogen atom, or to a monovalent side group, the monovalent side group is a side chain comprising the second drug unit, or a terminal group including no drug unit;
the terminal group at each occurrence is independently selected from the group consisting of C₁₋₆ alkyl, -L_{A}-COOH, -L_{A}-NH₂, -L_{A}-OH, -L_{A}-SH, -L_{A}-CONH₂, -L_{A}-imidazolyl, -L_{A}-NHC(═NH)NH₂, -L_{A}-phenyl, -L_{A}-indolyl, and -L_{A}-S—C₁₋₃ alkyl; L_{A} is each independently C₁₋₆ alkylene.

51. The drug-loaded monomolecular nano-polymer according to claim 48, wherein in each molecule of the drug-loaded monomolecular nano-polymer, a percentage by number of the platinum atoms in the divalent linking groups relative to a total number of amino acid units is in a range from 10% to 100%.

52. The drug-loaded monomolecular nano-polymer according to claim 49, comprising a tetravalent structural unit represented by formula (I), a monovalent structural unit represented by formula (III), a bivalent structural unit represented by formula (II), and a bivalent structural unit represented by formula (IV), 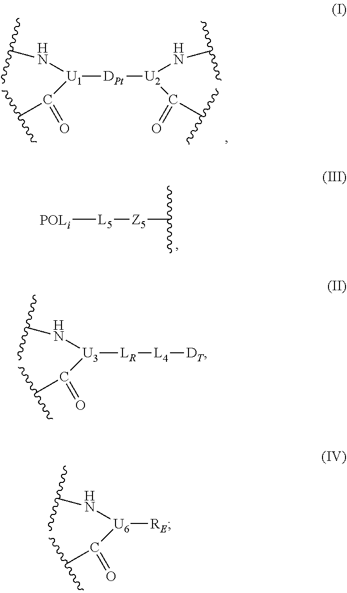
wherein in formula (I) at each occurrence, U₁ and U₂ are each independently a carbon-centered trivalent group, and D_{Pt} is the platinum-based drug unit;
in formula (III) at each occurrence, POL₁ is the hydrophilic polymer chain, L₅ is independently divalent or absent, and Z₅ is independently —NH—, or —C(═O)—;
in formula (II) at each occurrence, U₃ is independently a carbon-centered trivalent group, L_{R} is independently the responsive linking group, L₄ is independently divalent or absent, and D_{T} is the second drug unit;
in formula (IV) at each occurrence, U₆ is independently a carbon centered trivalent group, and R_{E} is independently H or R₀, R₀ is a terminal group including no drug unit.

53. The drug-loaded monomolecular nano-polymer according to claim 52, wherein 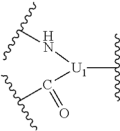
at each occurrence independently comprises 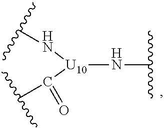
wherein U₁₀ is independently a trivalent hydrocarbon group; 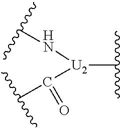
at each occurrence independently comprises 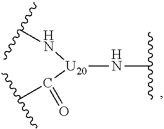
wherein U₂₀ is independently a trivalent hydrocarbon group.

54. The drug-loaded monomolecular nano-polymer according to claim 52, wherein D_{Pt} and adjacent groups form a main chain having a structure —C(═O)—O—Pt—O—C(═O)—, or —C(═O)—NH—O—Pt—O—NH—C(═O)—.

55. The drug-loaded monomolecular nano-polymer according to claim 52, wherein formula (I) at each occurrence independently comprises a structure represented by formula (I-1): 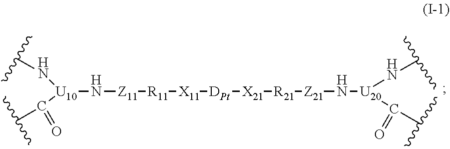
wherein U₁₀ and U₂₀ are each independently a trivalent hydrocarbon group;
Z₁₁ and Z₂₁ are each independently absent, or —C(═O)—, or —C(═O)—O—*, where “*” points to D_{Pt};
R₁₁ and R₂₁ are each independently C₁₋₆, hydrocarbylene;
X₁₁ and X₂₁ are each independently —C(═O)—O—*, or —C(═O)—NH—O—*, where “*” points to D_{Pt}.

56. The drug-loaded monomolecular nano-polymer according to claim 52, wherein D_{Pt} at each occurrence is independently a residue of a compound selected from the group consisting of cisplatin, carboplatin, nedaplatin, oxaliplatin, and lobaplatin.

57. The drug-loaded monomolecular nano-polymer according to claim 52, wherein 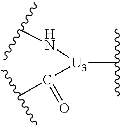
at each occurrence independently comprises 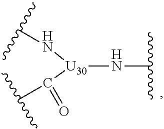
wherein U₃₀ is independently a trivalent hydrocarbon group.

58. The drug-loaded monomolecular nano-polymer according to claim 52, wherein L_{R} at each occurrence independently comprises a linking group capable of having bond cleavage under a condition selected from the group consisting of an intracellular reducing condition, a reactive oxygen species condition, a pH condition, an enzymatic lysis condition, a hydrolysis condition, and any combination thereof;
the pH condition is a pH value less than 6.8;
the enzymatic lysis condition comprises one or both of MMP-2 enzyme and azoreductase;
the hydrolysis condition is an acidic hydrolysis condition or an alkaline hydrolysis condition.

59. The drug-loaded monomolecular nano-polymer according to claim 52, wherein L_{R} at each occurrence independently comprises one or more linking groups selected from the group consisting of:
—S—S—,
an oxalate group, a borate group, a thioketal group, a thioether group, a mono-selenium group, a diselenium group, a divalent tellurium group, a thiazolinone group, a boronic acid group, an oligomeric chain of 3 to 7 prolines,
an acetal group, a hydrazone bond,
a peptide GPLGVRG, an azo group,
—C(═O)—O—, and —O—C(═O)—.

60. The drug-loaded monomolecular nano-polymer according to claim 52, wherein -L₄-D_{T} at each occurrence independently comprises Z₄-D_{T}, and Z₄ at each occurrence is independently a chemical bond, or selected from the group consisting of —C(═O)—, —O—, —S—, —O—C(═O)—*, —NH—C(═O)—*, and —NH—, where “*” points to D_{T}.

61. The drug-loaded monomolecular nano-polymer according to claim 52, wherein formula (II) at each occurrence has a structure represented by formula (II-1): 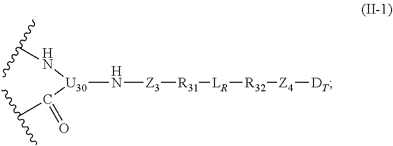
wherein U₃₀ at each occurrence is independently a trivalent hydrocarbon group;
R₃₂ at each occurrence is independently C₁₋₆ hydrocarbylene;
Z₄ at each occurrence is independently a chemical bond, or selected from the group consisting of —C(═O)—, —O—, —S—, —O—C(═O)—*, —NH—C(═O)—*, and —NH—, where “*” points to D_{T};
Z₃ at each occurrence is independently absent, —C(═O)—, or —C(═O)—O—*; where “*” points to R31;
R₃₁ at each occurrence is independently C₁₋₆ hydrocarbylene.

62. The drug-loaded monomolecular nano-polymer according to claim 52, wherein D_{T} at each occurrence is independently a residue of a compound selected from the group consisting of a camptothecin-based compound, resiquimod, and paclitaxel.

63. The drug-loaded monomolecular nano-polymer according to claim 52, wherein Z₅ at each occurrence is independently —NH—, —C(═O)—, or *—O—C(═O)—, where “*” points to L₅;
L₅ at each occurrence is independently C₁₋₆ hydrocarbylene.

64. The drug-loaded monomolecular nano-polymer according to claim 52, wherein POLᵢ at each occurrence is independently selected from the group consisting of polyethylene glycol, poly(propylene glycol), a copolymer of ethylene glycol and propylene glycol, poly(ethoxylated polyol), poly(alkenol), poly(vinylpyrrolidone), poly(hydroxyalkyl methacrylamide), poly(hydroxyalkyl methacrylate), polysaccharide, poly(α-hydroxyacid), poly(vinyl alcohol), polyphosphazene, polyoxazoline, poly(N-acryloylmorpholine), and any combination thereof;
a molecular weight of the hydrophilic polymer chain is in a range from 50 Da to 100 kDa.

65. The drug-loaded monomolecular nano-polymer according to claim 52, wherein formula (I) at each occurrence is independently a tetravalent structural unit represented by formula (I-2), formula (III) at each occurrence is independently a monovalent structural unit represented by formula (III-2), formula (II) at each occurrence is independently a bivalent structural unit represented by formula (II-2), and formula (IV) at each occurrence is independently a bivalent structural unit represented by formula (IV-1), 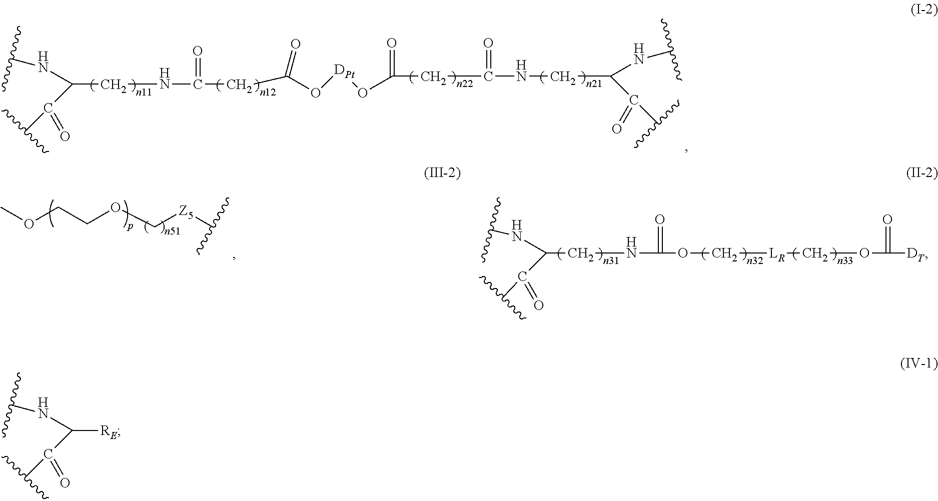
n11 and n21 are each independently 3 or 4; n12 and n22 are each independently 1, 2, 3, 4, or 5;
n31 is independently 3 or 4; n32 is independently 2, 3 or 4; n33 is independently 2, 3 or 4;
n51 is independently 1, 2, 3, or 4;
p is independently a positive integer less than or equal to 2500;
R_{E} is independently H or R₀, R₀ is the terminal group including no drug unit; R₀ at each occurrence is independently selected from the group consisting of C₁₋₆ alkyl, -L_{A}-COOH, -L_{A}-NH₂, -L_{A}-OH, -L_{A}-SH, -L_{A}-CONH₂, -L_{A}-imidazolyl, -L_{A}-NHC(═NH)NH₂, -L_{A}-phenyl, -L_{A}-indolyl, and -L_{A}-S—C₁₋₃ alkyl; L_{A} is each independently C₁₋₆ alkylene;
D_{Pt} is a residue of a compound selected from the group consisting of cisplatin, oxaliplatin, and 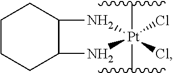
D_{T} is a residue of a compound selected from the group consisting of camptothecin, paclitaxel, and resiquimod.

66. The drug-loaded monomolecular nano-polymer according to claim 48, having a weight average molecular weight in a range from 200 kDa to 5000 kDa;
in each molecule of the drug-loaded monomolecular nano-polymer, the number of platinum atoms is greater than 40.

67. A drug-loaded monomolecular nano-polymer micelle, comprising the drug-loaded monomolecular nano-polymer of claim 48,
wherein the drug-loaded monomolecular nano-polymer micelle has a core-shell structure, the shell is a hydrophilic layer made of the at least one hydrophilic polymer chain, and the platinum-based drug unit is encapsulated in the core;
a particle size of the micelle in water at 25° C. is in a range from 10 nm to 120 nm;
an average diameter of the micelle is in a range from 15 nm to 50 nm;
a radius of the core of the micelle in water at 25° C. is in a range from 5 nm to 50 nm;
a thickness of the shell of the micelle in water at 25° C. is in a range from 5 nm to 40 nm.
